(19) United States
(12) Patent Application Publication
(10) Pub. No.: US 2026/0008757 A1
(43) Pub. Date: Jan. 8, 2026

(54) N-(3-(AMINOMETHYL)-PHENYL)-5-(4-PHENYL)-5-(TRIFLUOROMETHYL)-4,5-DIHYDROISOXAZOL-3-AMINE DERIVATIVES AND SIMILAR COMPOUNDS AS PESTICIDES

(71) Applicant: BASF SE, Ludwigshafen (DE)

(72) Inventors: Nikolas Huwyler, Ludwigshafen (DE); Karsten Koerber, Ludwigshafen (DE); Sebastian Armin Schwengers, Ludwigshafen (DE)

(21) Appl. No.: 19/112,425

(22) PCT Filed: Sep. 11, 2023
(86) PCT No.: PCT/EP2023/074841

§ 371 Date: Mar. 17, 2025

(30) Foreign Application Priority Data
Sep. 20, 2022 (EP) 22196624.5
Sep. 20, 2022 (EP) 22196626.0

(51) Int. Cl. C07D 261/04; A01N 43/80; A01P 7/04; A61P 43/00; C07D 413/12

(52) U.S. Cl. CPC C07D 261/04; A01N 43/80; A01P 7/04; A61P 43/00; C07D 413/12

(57) ABSTRACT

The invention relates to isoxazoline compounds of formula I wherein the variables have the meanings as defined in the specification, to compositions comprising them, to active compound combinations comprising them, and to their use for protecting growing plants and animals from attack or infestation by invertebrate pests, furthermore, to seed comprising such compounds. Preferred compounds are e.g. N-(3-(aminomethyl)-4-fluorophenyl)-5-(4-fluo-rophenyl)-5-(trifluoromethyl)-4,5-dihydroisoxazol-3-amine derivatives.

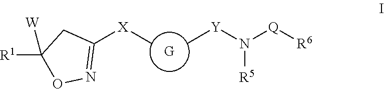

The invention relates to isoxazoline compounds of formula I

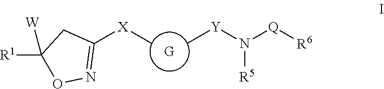

wherein
R¹ is C₁-C₂-haloalkyl;
W is phenyl, or pyridyl; wherein W is unsubstituted, partially or fully substituted with R²;
   R² is halogen, OR²¹, NR²²R²³, CN, NO₂, Si(CH₃)₃, SF₅, C₁-C₄-alkyl, C₁-C₄-haloalkyl, C₃-C₆-cycloalkyl, C₃-C₆-halocycloalkyl, C₂-C₄-alkenyl, C₂-C₄-haloalkenyl, C₂-C₄-alkynyl, C₂-C₄-haloalkynyl, C₁-C₃-alkoxy, C₁-C₃-haloalkoxy, C₁-C₃-alkyl-S(O)ₘ, C₁-C₃-haloal-kyl-S(O)ₘ, C₃-C₆-cycloalkyl-S(O)ₘ, C₁-C₃-alkoxy-C₁-C₄-alkyl, C₁-C₃-haloalkoxy-C₁-C₄-alkyl, C₁-C₃-alkyl-S(O)ₘ—C₁-C₄-alkyl, C₁-C₃-haloalkyl-S(O)ₘ—C₁-C₄-alkyl; which groups are unsubstituted, partially or fully substituted with R²¹¹;
   R²¹ H, C₁-C₆-alkyl, C₂-C₆-alkenyl, C₂-C₆-alkynyl, C₃-C₈-cycloalkyl, Si(C₁-C₄-alkyl)₃, C₁-C₃-alkyl-S(O)ₘ, C₃-C₆-cycloalkyl-S(O)ₘ, S(O)ₘR²⁴, which groups are unsubstituted, partially or fully substituted with R²¹¹;
   R²², R²³ H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, which are unsubstituted or partially or fully substituted with R²²¹; or
      C₁-C₆-alkyl-C(═O)OR²⁴, C₁-C₆-alkyl-C(═U)N(R^{25a})R^{25b},
      C₁-C₆-alkyl-C(═NR²⁵)N(R^{25a})R^{25b}, S(O)ₘR²⁴, S(O)ₘN(R^{25a})R^{25b}, C(═U)R²⁶,
      C(═O)OR²⁴, C(═U)N(R^{25a})R^{25b},
      C(═S)SR²⁴, C(═NR²⁵)R²⁶;
      C₃-C₈-cycloalkyl, C₃-C₈-halocycloalkyl;
   R²² and R²³ form together with the nitrogen atom they are bonded to a 3-, 4-, 5-, or 6-membered fully unsaturated heterocycle, which heterocycle may additionally contain one heteroatom selected from N, O, and S(O)ₘ as ring members, and which heterocycle is unsubstituted or partially or fully substituted with R²²²; or
   R²² and R²³ together form a group ═C(R²⁶)₂, ═S(O)ₘ(R²⁴)₂, ═S(O)ₘR²⁴N(R^{25a})R^{25b}; U is O or S;
   R²⁴ is H, Si(C₁-C₄-alkyl)₃, C₁-C₆-alkyl, C₂-C₆-alkenyl, C₂-C₆-alkynyl, which are unsubstituted or partially or fully halogenated and/or substituted with C₃-C₄-cycloalkyl, C₁-C₄-alkoxy, C₁-C₄-haloalkoxy, S(O)ₘ—C₁-C₄-alkyl, and oxo;
      C₃-C₈-cycloalkyl which is unsubstituted or partially or fully halogenated and/or substituted with C₁-C₄-alkyl, C₃-C₄-cycloalkyl, C₁-C₄-alkoxy, C₁-C₄-haloalkoxy, S(O)ₘ—C₁-C₄-alkyl, and oxo;
      phenyl, benzyl, pyridyl and phenoxy, which are unsubstituted or partially or fully halogenated and/or substituted with C₁-C₆-alkyl, C₁-C₆-haloalkyl, C₁-C₆-alkoxy, C₁-C₆-haloalkoxy, C₁-C₆-alkylthio, C₁-C₆-haloalkylthio, and (C₁-C₆-alkoxy)carbonyl;
   R²⁵ is H, C₁-C₆-alkoxy, C₁-C₆-haloalkoxy, C(═U)R²⁶, C(═O)OR²⁴, C(═O)NH(C₁-C₄-alkyl),
      C(═O)N(C₁-C₄-alkyl)₂, S(O)ₘR²⁴, S(O)ₘ—C₁-C₄-alkyl, S(O)ₘ—C₁-C₄-haloalkyl, S(O)ₘ—C₃-C₆-cycloalkyl, Si(C₁-C₄-alkyl)₃, NR²⁴C(═O)—C₁-C₄-alkyl, NR²⁴C(═O)—C₃-C₆-cycloalkylalkyl, CR²⁴N═OR²⁴, CR²⁶N═OR²⁴,
      C₁-C₆-alkyl, C₃-C₆-alkenyl, C₃-C₆-alkynyl, which are unsubstituted or partially or fully halogenated and/or substituted with CN, C₁-C₄-alkoxy, C₁-C₄-haloalkoxy, S(O)ₘ—C₁-C₄-alkyl, S(O)ₘ—C₁-C₄-haloalkyl, C₃-C₆-cycloalkyl which is unsubstituted or substituted with 1 or 2 halogen and/or CN; phenyl, or 4-, 5-, 6-membered saturated, partially or fully unsaturated heterocyclyl comprising 1, 2 or 3 heteroatoms selected from N, O, and S(O)ₘ as ring members, which rings are unsubstituted or partially or fully substituted with halogen, CN, C₁-C₄-alkyl, C₁-C₄-haloalkyl, C₁-C₃-alkoxy, C₁-C₃-haloalkoxy, C₁-C₂-alkyl-C₁-C₂-alkoxy, C₁-C₃-alkylthio, C₁-C₃-haloalkylthio, C(═O)—C₁-C₄-alkoxy; and oxo;
      C₃-C₈-cycloalkyl which is unsubstituted or partially or fully halogenated and/or substituted with CN, C₁-C₄-alkyl, C₁-C₄-alkoxy, C₁-C₄-haloalkyl, C₁-C₄-haloalkoxy, S(O)ₘ—C₁-C₄-alkyl, S(O)ₘ—C₁-C₄-haloalkyl, C(═O)NH(C₁-C₄-alkyl), C(═O)N(C₁-C₄-alkyl)₂, phenyl, or 5-, 6-membered heteroaryl containing 1, 2 or 3 heteroatoms selected from N, O, and S(O)ₘ as ring members, which rings are unsubstituted or partially or fully substituted with halogen, CN, C₁-C₄-alkyl, C₁-C₄-haloalkyl, C₁-C₃-alkoxy, C₁-C₃-haloalkoxy, C₁-C₂-alkyl-C₁-C₂-alkoxy, C₁-C₃-alkylthio, C₁-C₃-haloalkylthio;
      phenyl, benzyl, phenoxy, 4-, 5-, 6-membered saturated, partially or fully unsaturated heterocyclyl comprising 1, 2 or 3 heteroatoms selected from N, O, and S(O)ₘ as ring members,
   which groups are unsubstituted or partially or fully halogenated and/or substituted with halogen, CN, NO₂, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, and (C₁-C₆-alkoxy)carbonyl; and a 3-, 4-, 5- or 6-membered saturated, partially or fully unsaturated heterocycle comprising 1, 2 or 3 heteroatoms selected from N, O, and S(O)ₘ as ring members, where the heterocycle is optionally substituted with one or more R²²²;
   R^{25a} and R^{25b} have the meanings given for R²⁵; or
   R^{25a} and R^{25b} present on the same nitrogen atom may together form ═C(R²⁶)₂, ═S(O)ₘ(R²⁴)₂, or ═S(O)ₘR²⁴N(R^{25a})R^{25b}; or
   R^{25a} and R^{25b}, together with the nitrogen atom to which they are bound, form a 3-, 4-, 5-, 6- or 7-membered saturated, partially unsaturated or fully unsaturated heterocyclic ring, wherein the heterocyclic ring may additionally contain 1 or 2 heteroatoms or heteroatom groups selected from N, O, and S(O)ₘ as ring members, which heterocycle is unsubstituted or substituted with one or more substituents halogen, CN, C₁-C₄-alkyl, C₁-C₄-haloalkyl, C₁-C₄-alkoxy, C₁-C₄-haloalkoxy, and oxo; or R^{25a} and R^{25b}, together with the nitrogen atoms to which they are bound in the group C(═NR²⁵)N(R^{25a})R^{25b}, form a 3-, 4-, 5-, 6- or 7-membered saturated, partially unsaturated or fully unsaturated heterocyclic ring, wherein the heterocyclic ring may additionally contain 1 or 2 heteroatoms or heteroatom groups selected from N, O, and S(O)ₘ as ring members, which heterocycle is unsubstituted or substituted with one or more substituents halogen, CN, C₁-C₄-alkyl, C₁-C₄-haloalkyl, C₁-C₄-alkoxy, C₁-C₄-haloalkoxy, and oxo;
   R²⁶ is H, CN, OH, SH, C₁-C₆-alkyl, C₂-C₆-alkenyl, C₂-C₆-alkynyl, C₃-C₈-cycloalkyl, which are unsubstituted, partially or fully halogenated and/or substituted with 1, 2, or 3 groups CN, C₁-C₄-alkyl, C₃-C₄-cycloalkyl, C₁-C₄-haloalkyl, C₁-C₄-alkoxy, C₁-C₄-haloalkoxy, S(O)ₘ—C₁-C₄-alkyl, S(O)ₘ—C₁-C₄-haloalkyl, phenyl, or 3-, 4-, 5-, 6- or 7-membered saturated, partially or fully unsaturated heterocycle containing 1, 2 or 3 heteroatoms N, O, and S(O)ₘ as ring members, which rings are unsubstituted or partially or fully substituted with halogen, CN, C₁-C₄-alkyl, C₁-C₄-haloalkyl, C₁-C₃-alkoxy, C₁-C₃-haloalkoxy, C₁-C₂-alkyl-C₁-C₂-alkoxy, C₁-C₃-alkylthio, C₁-C₃-haloalkylthio; and oxo;
      C₁-C₆-alkoxy, C₁-C₆-haloalkoxy, S(O)ₘ—C₁-C₆-alkyl, S(O)ₘ-C₁-C₆-haloalkyl, Si(C₁-C₄-alkyl)₃, C(═O)N(R^{25a})R^{25b},
      phenyl, benzyl, phenoxy, a 3-, 4-, 5-, 6- or 7-membered saturated, partially or fully unsaturated heterocycle containing 1, 2 or 3 heteroatoms selected from N, O, and S(O)ₘ as ring members, which rings are unsubstituted or partially or fully halogenated and/or substituted with R²²²;
   R²¹¹ halogen, CN, C₁-C₄-alkoxy, C₁-C₄-haloalkoxy, C₃-C₄-alkenyloxy, C₃-C₄-haloalkenyloxy, C₃-C₄-alkynyloxy, C₃-C₄-haloalkynyloxy, C₁-C₄-alkyl-S(O)ₘ, C₁-C₄-haloalkyl-S(O)ₘ, C₃-C₄-alkenyl-S(O)ₘ, C₃-C₄-haloalkenyl-S(O)ₘ, C₃-C₄-alkynyl-S(O)ₘ, C₃-C₄-haloalkynyl-S(O)ₘ, and oxo;
      C₃-C₈-cycloalkyl, C₃-C₈-halocycloalkyl, C₃-C₈-cycloalkenyl, C₃-C₈-halocycloalkenyl;
   R²²¹ CN, NO₂, OH, SH, SCN, SF₅, Si(C₁-C₄-alkyl)₃, C₁-C₆-alkoxy, C₁-C₆-haloalkoxy, C₁-C₆-alkyl-S(O)ₘ, C₁-C₆-haloalkyl-S(O)ₘ, C(═O)N(R^{25a})R^{25b};
      C₃-C₈-cycloalkyl which is unsubstituted, partially or fully halogenated and/or partially or fully substituted with C₁-C₄-alkyl, C₃-C₄-cycloalkyl, C₁-C₄-alkoxy, C₁-C₄-haloalkoxy, and oxo; or
   two R²²¹ present on the same carbon atom of an alkyl, alkenyl, alkynyl or cycloalkyl group may together be ═O, ═CH(C₁-C₄-alkyl), ═C(C₁-C₄-alkyl)₂, ═N(C₁-C₆-alkyl), or ═NO(C₁-C₆-alkyl);
   R²²² is halogen, NO₂, CN, OH, SH, C₁-C₆-alkoxy, C₁-C₆-haloalkoxy, S(O)ₘ—C₁-C₄-alkyl, S(O)ₘ—C₁-C₄-haloalkyl, C₁-C₄-alkylcarbonyl, C₁-C₄-haloalkylcarbonyl, C₁-C₄-alkoxycarbonyl, C₁-C₄-haloalkoxycarbonyl, N(R^{25a})R^{25b}, C(═O)NR^{25a}R^{25b}, Si(C₁-C₄-alkyl)₃; C₁-C₄-alkyl, C₂-C₆-alkenyl, C₂-C₆-alkynyl, which are unsubstituted or partially or fully halogenated and/or substituted with CN, C₃-C₄-cycloalkyl, C₁-C₄-alkoxy, C₁-C₄-haloalkoxy, S(O)ₘ—C₁-C₄-alkyl, and oxo;
      C₃-C₈-cycloalkyl which is unsubstituted or partially or fully halogenated and/or substituted with CN, C₁-C₄-alkyl, C₃-C₄-cycloalkyl, C₁-C₄-alkoxy, C₁-C₄-haloalkyl, C₁-C₄-haloalkoxy, S(O)ₘ—C₁-C₄-alkyl, and oxo; or
   two R²²² present together on the same atom of an unsaturated or partially unsaturated ring may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl; or
   two R²²² on two adjacent carbon atoms form together with the carbon atoms they are bonded to a 4-, 5-, 6-, 7- or 8-membered saturated, partially unsaturated or maximally unsaturated ring, wherein the ring may contain 1 or 2 heteroatoms or heteroatom groups selected from N, O, and S(O)ₘ as ring members, and wherein the ring is optionally substituted with one or more groups C₁-C₄-alkyl, C₁-C₄-haloalkyl, C₁-C₄-alkoxy, and/or C₁-C₄-haloalkoxy;
   m is 0, 1, or 2;
   X is NR³, or O;
   R³ is H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, OR²¹, C₁-C₆-alkylthio, C₁-C₆-haloalkylthio, which are unsubstituted or partially or fully substituted with R³¹; or
      C₁-C₆-alkyl-C(═O)OR²⁴, C₁-C₆-alkyl-C(═U)N(R^{25a})R^{25b},
      C₁-C₆-alkyl-C(═NR²⁵)N(R^{25a})R^{25b}, C₁-C₆-alkyl-OC(═O)OR²⁴, N(R^{25a})R^{25b}, S(O)ₘR²⁴ S(O)ₘN(R^{25a})R^{25b}, C(═U)R²⁶, C(═O)OR²⁴, C(═U)N(R^{25a})R^{25b}, C(═S)SR²⁴,
      C(═NR²⁵)R²⁶;
      C₃-C₈-cycloalkyl, C₃-C₈-halocycloalkyl, phenyl, a 3-, 4-, 5-, 6- or 7-membered saturated or partially unsaturated heterocycle comprising 1, 2, 3 or 4 heteroatoms selected from N, O, and S(O)ₘ as ring members, or a 5- or 6-membered hetaryl comprising 1, 2, 3 or 4 heteroatoms selected from N, O, and S(O)ₘ as ring members, which rings are unsubstituted or partially or fully substituted with R³²;
   R³¹ halogen, CN, NO₂, OH, SH, SCN, SF₅, Si(C₁-C₄-alkyl)₃, N(R^{25a})R²⁵, C₁-C₆-alkoxy, C₁-C₆-haloalkoxy, C₁-C₆-alkyl-S(O)ₘ, C₁-C₆-haloalkyl-S(O)ₘ, C(═O)N(R^{25a})R^{25b};
      C₃-C₈-cycloalkyl which is unsubstituted, partially or fully halogenated and/or partially or fully substituted with CN, C₁-C₄-alkyl, C₃-C₄-cycloalkyl, C₁-C₄-alkoxy, C₁-C₄-haloalkyl, C₁-C₄-haloalkoxy, and oxo;
      S(O)ₘR²⁴, S(O)ₘN(R^{25a})R^{25b}, C(═U)R²⁶, C(═O)OR²⁴, C(═U)N(R^{25a})R^{25b}, C(═S)SR²⁴, C(═NR²⁵)R²⁶;
      phenyl, benzyl, phenoxy, or 3-, 4-, 5-, 6- or 7-membered saturated, partially, or fully unsaturated heterocycle containing 1, 2, or 3 heteroatoms selected from N, O, S(O)ₘ as ring members, wherein the rings are unsubstituted or partially or fully substituted with R²²²; or
      two R³¹ present on the same carbon atom of an alkyl, alkenyl, alkynyl or cycloalkyl group may together be ═O, ═CH(C₁-C₄-alkyl), ═C(C₁-C₄-alkyl)₂, ═N(C₁-C₆-alkyl), or ═NO(C₁-C₆-alkyl);
   R³² is a group as defined in R³¹ or selected from C₁-C₆-alkyl, C₂-C₆-alkenyl, and C₂-C₆-alkynyl, which groups are unsubstituted, partially or fully halogenated and/or substituted with one or two CN, C₃-C₄-cycloalkyl, C₁-C₄-alkoxy, C₁-C₄-haloalkoxy, or oxo;
G is phenyl, or 6-membered heteroaryl comprising as ring members 1 or 2 N-atoms; wherein G is unsubstituted, partially, or fully substituted with R⁴;
   R⁴ is as defined for R²;
R⁵ is a group as defined for R³;
Y is a direct bond or C(R^{4a})(R^{4b});
   R^{4a} and R^{4b} are H, halogen, CN, NO₂, C₁-C₄-alkyl, C₁-C₄-haloalkyl, C₃-C₆-cycloalkyl unsubstituted or substituted with CN or halogen;
   R⁴ and R^{4a} and/or R^{4b} together form a 3-, 4-, 5-, 6- or 7-membered saturated, partially, or fully unsaturated heterocycle, which may contain 1 or 2 heteroatoms selected from N, O, and S(O)ₘ as ring members, and wherein the ring is unsubstituted or substituted with one or more groups R²;
Q is C(═U) or S(O)ₘ;
R⁶ is as defined for R²⁵; or
   R⁵ and R⁶ together form a 4-, 5-, 6- or 7-membered saturated, or partially unsaturated heterocycle, which additionally to the N(R⁵) may contain 1 or 2 heteroatoms selected from N, O, and S(O)ₘ as ring members, and wherein the ring is unsubstituted or substituted with one or more groups R²;
the N-oxides, stereoisomers and agriculturally or veterinarily acceptable salts thereof.

The invention also provides an agricultural composition comprising at least one compound of formula I, a stereoisomer thereof and/or an agriculturally acceptable salt thereof and at least one liquid and/or solid carrier, especially at least one inert liquid and/or solid agriculturally acceptable carrier.

The invention also provides a veterinary composition comprising at least one compound of formula I, a stereoisomer thereof and/or a veterinarily acceptable salt thereof and at least one liquid and/or solid carrier, especially at least one inert veterinarily liquid and/or solid acceptable carrier.

The invention also provides a method for controlling invertebrate pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of formula I or a salt thereof as defined herein.

The present invention also relates to plant propagation material, in particular seed, comprising at least one compound of formula I and/or an agriculturally acceptable salt thereof.

The invention further relates to a method for treating or protecting an animal from infestation or infection by parasites which comprises bringing the animal in contact with a parasiticidally effective amount of a compound of formula I or a veterinarily acceptable salt thereof. Bringing the animal in contact with the compound I, its salt or the veterinary composition of the invention means applying or administering it to the animal.

WO2010/020522, WO2010/135360, WO2022/171472, and EP4043444 describe structurally closely related active compounds. These compounds are mentioned to be useful for combating invertebrate pests.

Nevertheless, there remains a need for highly effective and versatile agents for combating invertebrate pests. It is therefore an object of the present invention to provide compounds having a good pesticidal activity and showing a broad activity spectrum against a large number of different invertebrate pests, especially against difficult to control pests, such as insects.

It has been found that these objects can be achieved by compounds of formula I as depicted and defined below, and by their stereoisomers, salts, tautomers and N-oxides, in particular their agriculturally acceptable salts.

Compounds of formula I with X being NR³ (formula I.1) can be prepared by reaction of a compound of formula IIA.1 with a compound of formula IIIA, wherein X^{H} is a halogen, preferably a bromide or iodide, in a Buchwald-Hartwig reaction [cf. WO2022/171472, or WO2010/135360].

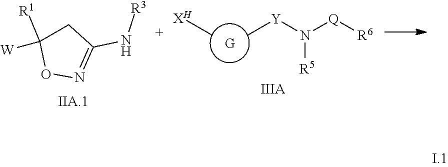

This transformation is usually carried out at temperatures from 20° C. to 180° C., preferably from 60° C. to 100° C., in an inert solvent, in the presence of a base and a palladium catalyst such as, e.g., tris(dibenzylideneacetone)dipalladium(0)/9,9-dimethyl-4,5-bis(diphenylphosphino)xanthene (Pd₂(dba)₃/XantPhos), [2-(di-tert-butylphosphino)-2′,4′,6′-triisopropyl-1,1′-biphenyl][2-(2-aminoethyl)-phenyl)]palladium(II) chloride, and the like [cf. WO2017/069980]. Suitable solvents are ethers such as 1,4-dioxane, THF, and the like; or alcohols such as butanol, tert-amyl alcohol, and the like; or polar aprotic solvents such as dimethylformamide (DMF), dimethylacetamide (DMA), N-methyl-2-pyrrolidon (NMP), dimethyl sulfoxide (DMSO), and the like. Preferably, 1,4-dioxane is employed. It is also possible to use mixtures of the solvents mentioned. Suitable bases are, in general, inorganic compounds, like alkali metal and alkaline earth metal carbonates, such as e.g. Li₂CO₃, K₂CO₃, Cs₂CO₃, CaCO₃, and the like; or organic compounds, such as alkali metal alcoholates, such as lithium tert-butylate, sodium tert-butylate, potassium tert-butylate, and the like. Particular preference is given to Cs₂CO₃. The bases are generally employed in equimolar amounts; however, they can also be used in in excess. The starting materials are generally reacted with one another in equimolar amounts. In terms of yield, it may be advantageous to employ an excess of IIA.1, based on IIIA.

Compounds of formula IIA.1 are known from the literature [cf. INV202944] and can be prepared as described therein.

Alternatively, compounds of formula I, can also be prepared by reaction of a compound of formula IIB, wherein X^{H} is a halogen, preferably a bromide or chloride, with a compound of formula IIIB in a base-promoted ipso-substitution reaction [cf. WO2022/171472].

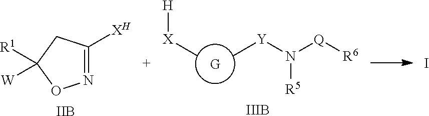

This transformation is usually carried out at temperatures of from 25° C. to 200° C., preferably from 60° C. to 150° C., in an inert solvent, in the presence of a base. Suitable solvents are DMF, DMA, NMP, DMSO, and the like; particular preference is given to NMP, and DMF. It is also possible to use mixtures of the solvents mentioned. Suitable bases are, in general, inorganic compounds, like alkali metal and alkaline earth metal hydroxides, such as e.g. LiOH, NaOH, KOH, Ca(OH)₂, and the like; or alkali metal and alkaline earth metal hydrides, such as e.g. LiH, NaH, KH, CaH₂, and the like; or alkali metal and alkaline earth metal carbonates, such as e.g. Li₂CO₃, K₂CO₃, CaCO₃, and the like; or organic bases, like N-containing heteroaromatics such as pyridine, 2,6-lutidine, and the like. The bases are generally employed in equimolar amounts; however, they can also be used in in excess or, if appropriate, as solvent.

Compounds of formula IIB can be prepared as described in literature [cf. WO2022/171472].

In addition, compounds of formula I.1 can be prepared by reaction of a compound of formula IIB, wherein X^{H} is a halogen, preferably a bromide or chloride, with an amine of formula IIIB.1 in an acid-promoted ipso-substitution reaction [cf. WO2022/171472].

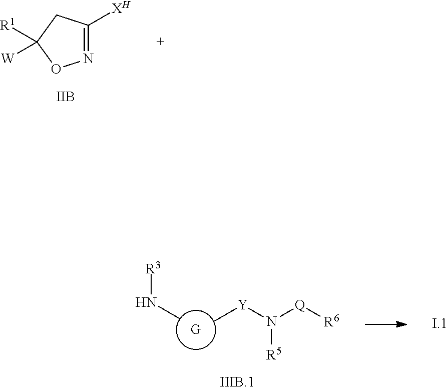

This transformation is usually carried out at temperatures of from 25° C. to 200° C., preferably from 60° C. to 150° C., in an inert solvent, in the presence of an acid. Suitable solvents are alcohols such as e.g. 2,4-dimethylpentan-3-ol, n-butanol, sec-butanol, tert-butanol, and the like; or aromatic hydrocarbons such as e.g. toluene, o-, m-, p-xylene, chlorobenzene, dichlorobenzene, and the like; or polar aprotic solvents such as e.g. DMSO, DMF, DMA, NMP, and the like; preferably alcohols such as 2,4-dimethylpentan-3-ol are employed. It is also possible to use mixtures of the solvents mentioned. Suitable acids and acidic catalysts are, in general, inorganic acids such as e.g. HCl, HBr, H₂SO₄, HClO₄, and the like; or organic acids such as e.g. toluene-sulfonic acid, benzenesulfonic acid, camphor sulfonic acid, trifluoromethanesulfonic acid, trifluoroacetic acid, and the like. The acids are generally employed in catalytic amounts; however, they can also be used in equimolar amounts, in excess or, if appropriate, as solvent. The starting materials are generally reacted with one another in equimolar amounts. In terms of yield, it may be advantageous to employ an excess of IIIB.1, based on IIB.

Furthermore, compounds of formula I can also be prepared by reaction of an olefin of formula VI with an in-situ formed compound of formula V, which in turn is derived from the reaction of a compound of formula IIIB with a dihaloaldoxime of formula IV, in a 1,3-dipolar cycloaddition reaction [cf. WO2022/171472].

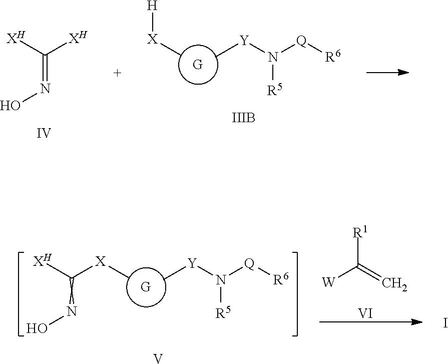

This transformation is usually carried out at temperatures from −78° C. to 80° C., preferably from −78° C. to 25° C., in the presence of a base. Suitable solvents are ethers such as e.g. THF, 1,4-dioxane, Et₂O, tert-butylmethylether, and the like; or esters such as e.g. EtOAc, and the like; or aromatic hydrocarbons such as e.g. benzene, toluene, o-, m-, and p-xylene, chlorobenzene, dichlorobenzene, and the like; or halogenated hydrocarbons such as, e.g. CH₂Cl₂, CHCl₃, 1,2-dichloroethane, and the like. It is also possible to use mixtures of the solvents mentioned. Suitable bases are, in general, organic bases like tertiary amines, such as e.g. triethylamine, diisopropylethylamine, and the like; or N-containing heteroaromatics such as e.g. pyridine, collidine, 2,6-lutidine, 4-dimethylaminopyridine, and the like; or inorganic compounds, like alkali metal and alkaline earth metal carbonates, such as e.g. Li₂CO₃, K₂CO₃, CaCO₃, and the like; or alkali metal bicarbonates, such as e.g. NaHCO₃, KHCO₃, and the like; or alkali metal phosphates such as e.g. K₃PO₄, and the like. The bases are generally employed in equimolar amounts; however, they can also be used in excess or, if appropriate, as solvent.

The starting materials are generally reacted with one another in equimolar amounts. In terms of yield, it may be advantageous to employ an excess of IV and IIIB, based on VI.

Compounds of formula IV are commercially available, and the preparation of compounds of formula VI are described in the literature [cf. WO2022/171472].

Compounds of formula IIIA wherein Y is a direct bond, can be prepared by reacting a compound of formula VII, wherein X^{LG} is a leaving group, preferably a fluorine or chlorine atom, with an amine of formula VIII in a base-promoted nucleophilic aromatic substitution reaction.

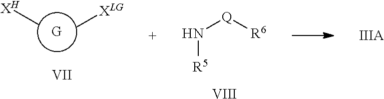

This reaction is usually carried out at temperatures from −20° C. to 180° C., preferably from 25° C. to 120° C., in an inert solvent, and in the presence of a base [cf. WO 2010/100189]. Suitable solvents are e.g. DMSO, DMF, DMA, NMP, and the like; or nitriles such as acetonitrile, propionitrile, and the like; or ethers such as 1,4-dioxane, THF, and the like; or aromatic hydrocarbons such as toluene, o-, m-, p-xylene, and the like; or alcohols such as methanol, ethanol, n-propanol, isopropanol, n-butanol, tert-butanol, and the like; or water. It is also possible to use mixtures of the solvents mentioned. Suitable bases are, in general, inorganic compounds, like alkali metal and alkaline earth metal carbonates, such as e.g. Li₂CO₃, K₂CO₃, Cs₂CO₃, CaCO₃, and the like; or alkali metal and alkaline earth metal hydrides, such as e.g. LiH, NaH, KH, CaH₂, and the like; or organic bases, like tertiary amines, such as e.g. trimethylamine, triethylamine, diisopropylethylamine, N-methylpiperidine, and the like; or N-containing aromatics such as e.g. pyridine, collidine, 2,6-lutidine, 4-dimethylaminopyridine, and the like; or alkali metal amides such as e.g. LiN(i-Pr)₂, LiN(SiMe₃)₂, NaN(SiMe₃)₃, KN(SiMe₃)₂, and the like; or alkali metal alcoholates such as potassium tert-butoxide, and the like. The bases are generally employed in equimolar amounts; however, they can also be used in in excess or, if appropriate, as solvent. The starting materials are generally reacted with one another in equimolar amounts. In terms of yield, it may be advantageous to employ an excess of VIII, based on VII.

Alternatively, it is also possible to prepare compounds of formula IIIA by reacting a compound of formula VII, wherein X^{LG} preferably is iodide, bromide, or triflate, with an N-heterocyclic compound of formula VIII in a palladium-catalyzed (i.e. Buchwald-Hartwig reaction) cross-coupling reaction.

Buchwald-Hartwig reactions are usually carried out at temperatures from 25° C. to 200° C., preferably from 50° C. to 150° C., in an inert solvent, and in the presence of a palladium catalyst and a base [cf. WO2016/168059]. Suitable solvents are e.g. aromatic hydrocarbons such as toluene, o-, m-, and p-xylene; or ethers such as 1,4-dioxane, and THF; or nitriles such as acetonitrile, and propionitrile; or polar aprotic solvents such as DMSO, DMF, DMA, NMP, and the like. Suitable palladium catalysts are e.g. Pd(OAc)₂/PPh₃, Pd(OAc)₂/2,2′-bis(diphenyl-phosphino)-1,1′-binaphthyl (BINAP), Pd(OAc)₂/4,5-bis(diphenylphosphino)-9,9-dimethyl-xanthene (XantPhos), dichloro[9,9-dimethyl-4,5-bis(diphenylphosphino)xanthene]palladium(II), [1,1′-bis(diphenylphosphino)ferrocene]dichloropalladium(II), (1,3-bis(diphenylphosphino)-propane)palladium(II) chloride, trans-bis(acetato)bis[o-(di-o-tolylphosphino)benzyl]dipalla-dium(II), [2-(di-tert-butylphosphino)-2′,4′,6′-triisopropyl-1,1′-biphenyl][2-(2-aminoethyl)-phenyl)]palladium(II) chloride, and the like. Suitable bases are, in general, inorganic compounds, like alkali metal and alkaline earth metal carbonates, such as e.g. Li₂CO₃, K₂CO₃, Cs₂CO₃, CaCO₃, and the like; or alkali metal phosphates such as e.g. K₃PO₄, and the like; or organic bases, like tertiary amines, such as e.g. triethylamine, diisopropylethylamine, N-methylpiperidine, N-methyl-N,N-dicyclohexylamine, and 1,4-diaza-bicyclo[2.2.2]octane; or amidines such as 1,8-diazabicyclo[5.4.0]undec-7-en; or alkali metal alcoholates such as sodium tert-butoxide; or alkali metal amides such as lithium bis(trimethylsilyl)amide (LiHMDS). The bases are generally employed in equimolar amounts; however, they can also be used in in excess or, if appropriate, as solvent.

Furthermore, it is also possible to prepare compounds of formula IIIA by reacting a compound of formula VII, wherein X^{LG} preferably is iodide, bromide, B(OH)₂, B(alkyl)₂, or B(O-alkyl)₂ with an N-heterocyclic compound of formula VIII in a copper-promoted (i.e. Ullmann or Chan-Lam-Evans reaction, resp.) cross-coupling reaction.

The copper-promoted reactions are usually carried out at temperatures from 25° C. to 200° C., preferably from 50° C. to 150° C., in an inert solvent, and in the presence of a copper catalyst and a base. Suitable solvents are e.g. N-containing aromatics such as pyridine, quinoline, and the like; or polar aprotic solvents such as DMSO, DMF, DMA, N-methyl-2-pyrrolidon (NMP), and the like; or ethers such as 1,4-dioxane, bis(2-methoxyethyl)ether, and the like; or aromatic hydrocarbons such as toluene, o-, m-, p-xylene, and the like; or nitriles such as acetonitrile, propionitrile, and the like; or alcohols such as methanol, isopropanol, tert-butanol, and the like; and it is also possible to use mixtures of the solvents mentioned. Suitable copper catalysts are e.g. CuI, CuBr, CuCl, Cu₂O, Cu(OAc)₂, and the like. If desired, it is also possible to employ an amine- or amide-based ligand in the reaction such as e.g. pyridine, 2,6-lutidine, 4-(dimethyl-amino)pyridine, quinoline, 1,10-phenantroline, and the like; or N,N,N′,N′-tetramethylethane-1,2-diamine, N,N-dimethylglycine, and the like. Suitable bases are, in general, organic bases, such as e.g. pyridine, 2,6-lutidine, and the like; or tertiary amines, such as triethylamine, and the like; or inorganic compounds, like alkali metal and alkaline earth metal carbonates, such as e.g. Li₂CO₃, K₂CO₃, Cs₂CO₃, CaCO₃, and the like; or alkali metal phosphates such as K₃PO₄, and the like. The bases are generally employed in equimolar amounts; however, they can also be used in in excess or, if appropriate, as solvent.

The starting materials are generally reacted with one another in equimolar amounts. In terms of yield, it may be advantageous to employ an excess of VIII, based on VII.

Aromatic compounds of formula VII and N-heterocyclic compounds of formula VIII are, in general, commercially available or known in the literature.

Compounds of formula IIIA wherein Y is C(R^{4a})(R^{4b}), can be prepared by reacting a compound of formula VIIa, wherein X^{LG} is a leaving group, preferably a fluorine or chlorine atom, with an amine of formula VIII in a nucleophilic substitution reaction.

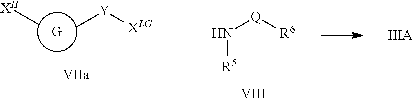

This transformation is usually carried out at temperatures of from −78° C. to +110° C., preferably from −20° C. to +80° C., in an inert solvent, in the presence of a base [cf. Journal of the Society of Chemical Industry, London, Transactions and Communications (1947), 66, 325].

Suitable solvents are aliphatic hydrocarbons such as pentane, hexane, cyclohexane, and petrol ether, aromatic hydrocarbons such as toluene, o-, m-, and p-xylene, halogenated hydrocarbons such as methylene chloride, chloroform, and chlorobenzene, ethers such as diethylether, diisopropylether, tert.-butylmethylether, dioxane, anisole, and tetrahydrofurane (THF), nitrils such as acetonitrile, and propionitrile, moreover, dimethyl formamide (DMF), and DMA, preferably aromatic hydrocarbons such as toluene; or DMF. It is also possible to use mixtures of the solvents mentioned.

Suitable bases are, in general, inorganic compounds, such as alkali metal and alkaline earth metal hydrides, such as LiH, NaH, KH, and CaH₂, alkali metal and alkaline earth metal carbonates, such as Li₂CO₃, K₂CO₃, and CaCO₃, and also alkali metal bicarbonates, such as NaHCO₃, moreover organic bases, e.g. tertiary amines, such as trimethylamine, triethylamine, triisopropylethylamine and N-methylpiperidine, pyridine, substituted pyridines, such as collidine, lutidine and 4-dimethylaminopyridine, and also bicyclic amines. Particular preference is given to alkali metal and alkaline earth metal carbonates such as potassium or sodium carbonate.

The bases are generally employed in catalytic amounts; however, they can also be used in equimolar amounts, in excess or, if appropriate, as solvent.

The starting materials are generally reacted with one another in equimolar amounts. In terms of yield, it may be advantageous to employ an excess of VIII, based on VIIa.

Compounds of formula IIIA wherein Y is C(R^{4a})(R^{4b}), can be prepared in a two-step protocol via compound XIa by reacting a compound of formula VIIb with an amine of formula VIIIa in a reductive amination reaction.

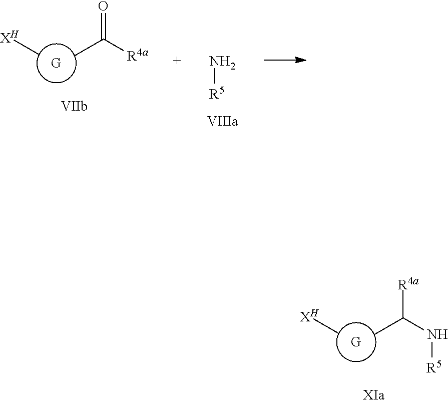

This transformation is usually carried out at temperatures of from −78° C. to +120° C., preferably from −20° C. to +25° C., in an inert solvent, in the presence of a reducing agent and an acid [cf. (literature) European Journal of Medicinal Chemistry (2018), 143, 390-401].

Suitable solvents are aliphatic hydrocarbons such as pentane, hexane, cyclohexane, and petrol ether, aromatic hydrocarbons such as toluene, o-, m-, and p-xylene, halogenated hydrocarbons such as methylene chloride, chloroform, and chlorobenzene, ethers such as diethylether, diisopropylether, tert.-butylmethylether, dioxane, anisole, and THF, alcohols such as methanol, ethanol, n-propanol, isopropanol, n-butanol, and tert.-butanol, preferably alcohols such as methanol, ethanol. It is also possible to use mixtures of the solvents mentioned.

Suitable acids and acidic catalysts are in general inorganic acids such as HF, HCl, HBr, H₂SO₄ and HClO₄, Lewis acids, such as BF₃, AlCl₃, FeCl₃, SnCl₄, TiCl₄, and ZnCl₂, moreover organic acids such as formic acid, acetic acid, propionic acid, oxalic acid, toluene sulphonic acid, benzene sulphonic acid, camphor sulphonic acid, citric acid, and trifluoro acetic acid. The acids are generally employed in catalytic amounts; however, they can also be used in equimolar amounts, in excess or, if appropriate, as solvent.

The starting materials are generally reacted with one another in equimolar amounts. In terms of yield, it may be advantageous to employ an excess of VIIIa, based on VIIb.

In a second step, compounds of formula XIa can be acylated with compounds of formula XIa to give compounds of formula IIIA.

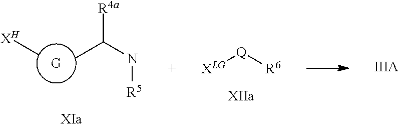

This transformation is usually carried out at temperatures of from −78° C. to +110° C., preferably from −20° C. to +110° C., in an inert solvent, in the presence of a base [cf. WO2007103905].

Suitable solvents are aliphatic hydrocarbons such as pentane, hexane, cyclohexane, and petrol ether, aromatic hydrocarbons such as toluene, o-, m-, and p-xylene, halogenated hydrocarbons such as methylene chloride, chloroform, and chlorobenzene, ethers such as diethylether, diisopropylether, tert.-butylmethylether, dioxane, anisole, and THF, nitrils such as acetonitrile, and propionitrile, moreover dimethyl sulphoxide, DMF, and DMA, preferably aromatic hydrocarbons, halogenated hydrocarbons and ethers such as toluene, methylene chloride and DMF. It is also possible to use mixtures of the solvents mentioned.

Suitable bases are, in general, inorganic compounds, such as alkali metal and alkaline earth metal hydrides, such as LiH, NaH, KH, and CaH₂, alkali metal and alkaline earth metal carbonates, such as Li₂CO₃, K₂CO₃, and CaCO₃, and also alkali metal bicarbonates, such as NaHCO₃, moreover organic bases, e.g. tertiary amines, such as trimethylamine, triethylamine, diisopropylethylamine, and N-methylpiperidine, pyridine, substituted pyridines, such as collidine, lutidine, and 4-dimethylaminopyridine, and also bicyclic amines. Particular preference is given to alkali metal carbonates and organic bases such as K₂CO₃, triethyl amine, or pyridine.

The bases are generally employed in catalytic amounts; however, they can also be used in equimolar amounts, in excess or, if appropriate, as solvent.

The starting materials are generally reacted with one another in equimolar amounts. In terms of yield, it may be advantageous to employ an excess of XIa, based on XIIa.

Compounds of formula IIIB.1, wherein X is NR³, can be prepared by reacting a compound of formula IIIA, wherein X^{H} is a halogen, preferably fluoride or chloride, with an amine R³NH₂ in a base-promoted nucleophilic aromatic substitution reaction under the same conditions as described above for the synthesis of IIIA from compounds of formula VII and VIII.

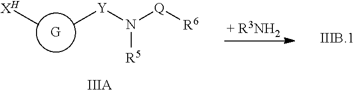

Alternatively, Compounds of formula IIIB.1, wherein X is NR³, can be prepared by reacting a compound of formula IIIA, wherein X^{H} is a halogen, preferably bromide or iodide, with an amine R³NH₂ in a Buchwald-Hartwig or Ullmann cross-coupling reaction, resp., under the same conditions as described above for the synthesis of IIIA from compounds of formula VII and VIII.

Furthermore, compounds of formula IIIB.1a, wherein X is NH, can be prepared from compounds of formula IX via reduction of the NO₂-group with SnCl₂, iron, or zinc, respectively.

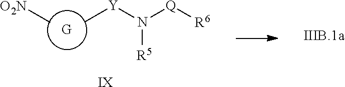

These reactions are usually carried out at temperatures from 25° C. to 180° C., preferably from 25° C. to 80° C., in an inert solvent, and in the presence of an acid. Suitable solvents are e.g. alcohols such as methanol, ethanol, isopropanol, n-butanol, and the like; or esters such as e.g. EtOAc, and the like; or ethers such as e.g. THF, and the like; or H₂O; and it is also possible to use mixtures of the solvents mentioned. Suitable acids are, in general, inorganic acids like mineral acids such as e.g. HCl, and the like; or ammonium salts such as e.g. NH₄Cl, and the like; or organic acids like carboxylic acids such as e.g. AcOH, and the like. The acids are generally employed in equimolar amounts; however, they can also be used in in excess or, if appropriate, as solvent. The starting materials are generally reacted with one another in equimolar amounts. In terms of yield, it may be advantageous to employ an excess of SnCl₂, iron, or zinc, resp., based on IX.

In addition, compounds of formula IIIB.1a, wherein X is NH, can also be prepared from compounds of formula IX via reduction of the NO₂-group with a Pd on activated carbon catalyst.

This reaction is usually carried out at temperatures from 0° C. to 100° C., preferably from 25° C. to 50° C., in an inert solvent, and under a 1-10 bar atmosphere of H₂. Suitable solvents are e.g. alcohols such as methanol, ethanol, isopropanol, n-butanol, and the like; or esters such as e.g. EtOAc, and the like; or ethers such as e.g. THF, and the like; or H₂O; and it is also possible to use mixtures of the solvents mentioned.

Compounds of formula IIIB.2, wherein X is O, can be prepared by reacting a compound of formula X, wherein X^{LG} is a leaving group, preferably iodide, bromide, triflate, B(OH)₂, B(alkyl)₂, or B(O-alkyl)₂, with an N-heterocyclic compound of formula VIII in a Buchwald-Hartwig, Ullmann, or Chan-Lam-Evans reaction, resp., under the same conditions as described above for the synthesis of IIIA from compounds of formula VII and VIII.

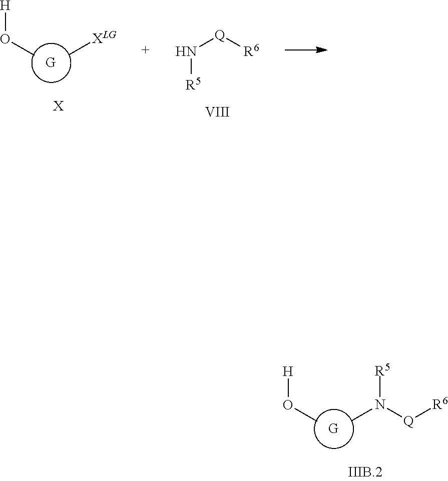

In turn, compounds of formula IX can be prepared by reacting a compound of formula XI, wherein X^{LG} is a leaving group, preferably fluoride or chloride, with an amine of formula VIII in a base-promoted nucleophilic aromatic substitution reaction under the same conditions as described above for the synthesis of IIIA from compounds of formula VII and VIII.

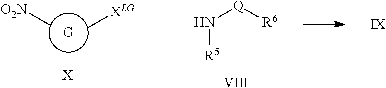

Alternatively, compounds of formula IX can be prepared by reacting a compound of formula XI, wherein X^{LG} is a leaving group, preferably iodide, bromide, triflate, B(OH)₂, B(alkyl)₂, or B(O-alkyl)₂, with an amine of formula VIII in a Buchwald-Hartwig, Ullmann, or Chan-Lam-Evans reaction, resp., under the same conditions as described above for the synthesis of IIIA from compounds of formula VII and VIII.

Compounds of formula XI are, in general, commercially available or known in the literature.

If desired, compounds of formula I.1, wherein R³ is not H, can also be prepared by alkylation or acylation of the corresponding H-compound (R³═H), analogously as previously described in the literature [cf. WO2022/171472].

Alternatively, compounds of formula I.1 can be prepared by reaction of a compound of formula IIA.1 with an acetal of formula IIIA.1, wherein X^{H} is a halogen, preferably a bromide or iodide, in a Buchwald-Hartwig reaction [cf. WO2022/171472, or WO2010/135360] as described in the outset to yield intermediate XI.1.

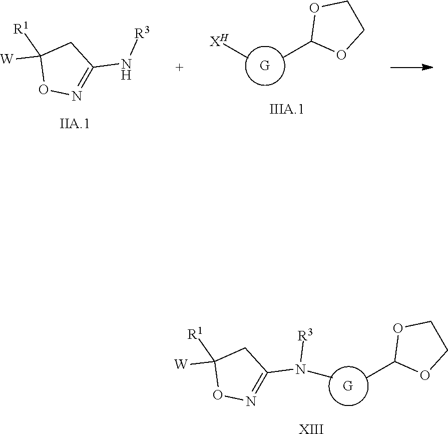

This transformation is usually carried out at temperatures of from 0° C. to 150° C., preferably from 80° C. to 120° C., in an inert solvent, in the presence of a base and a catalyst [cf. M. C. Harris, Journal of Organic Chemistry (1999), 64(16), 6019-6022].

Acetals IIIA.1 are commercially available or can be prepared by methods known in the art.

Suitable solvents are ethers such as diethylether, diisopropylether, tert.-butylmethylether, dioxane, anisole, and THF, preferably dioxane.

Acetals XIII are transformed to aldehydes XIV under acidic conditions.

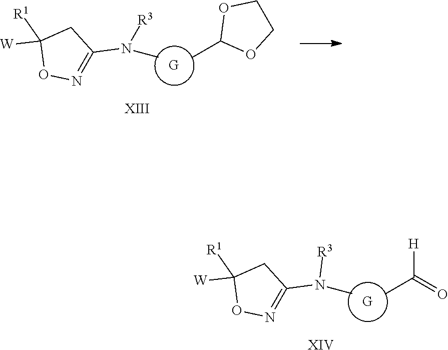

This transformation is usually carried out at temperatures of from 0° C. to +80° C., preferably from 10° C. to 50° C., in an inert solvent, in the presence of an acid [cf. X. Wu, Advanced Synthesis & Catalysis (2018), 360(6), 1111-1115].

Suitable solvents are ethers such as diethylether, diisopropylether, tert.-butylmethylether, dioxane, anisole, and THF, preferably THF. Suitable acids and acidic catalysts are in general anorganic acids such as HCl, HBr, H₂SO₄, and HClO₄, preferably HCl, in aqueous solution.

Aldehydes XIV are transformed to amines XV under reductive conditions with ammonia.

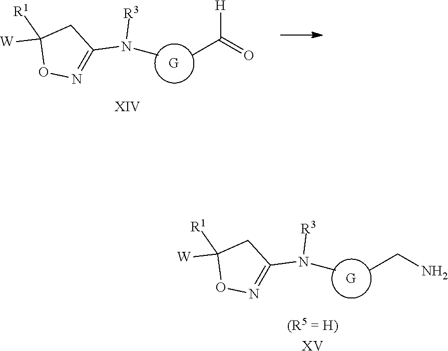

This transformation is usually carried out at temperatures of from 0° C. to 120° C., preferably from 0° C. to 50° C., in an inert solvent, in the presence of a reduction agent (e.g. NaBH₄) and NH₃, [cf. C. R. Reddy et al, Journal of Organic Chemistry (2023), 88(11), 7117-7127 (].

Suitable solvents are alcohols such as methanol, ethanol, n-propanol, isopropanol, n-butanol, and tert.-butanol, preferably methanol and ethanol. Suitable alternative solvents are, for example, organic acids such as acetic acid or mixtures of acetic acid and alcohols.

Amino compounds XV can be acylated with compounds of formula XIIa to give compounds I.1.

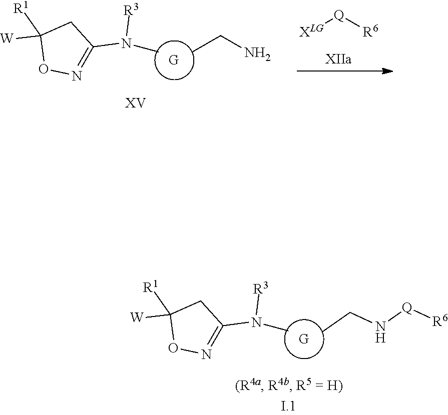

This transformation is usually carried out under conditions described above for the reaction of XIa with XIIa.

Alternatively, the reaction sequence above (IIIA.1→XIII→XIV→XV→I.1) can be run with compounds with R³ being H, and the introduction of R³ can be applied in a later stage, e.g. to compounds XIII, or I.1.

Alternatively, compounds of formula XV can be prepared by cleavage of a compound of formula I.1 wherein the group -Q-R⁶ represents a protective group, such as Boc, mesyl, or tosyl.

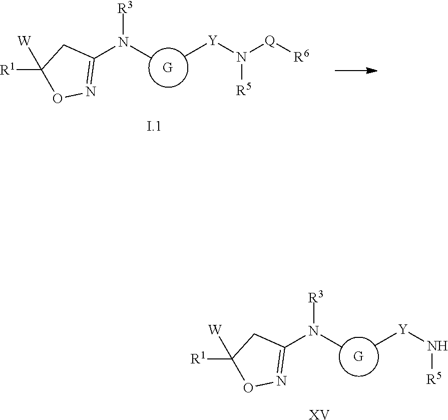

This transformation is usually carried out under acidic, protic conditions known in the art.

The starting materials required for preparing the compounds I are commercially available or known from the literature [cf. e.g. WO2022/171472] or can be prepared in accordance with the literature cited.

The reaction mixtures are worked up in a customary manner, for example by mixing with water, separating the phases and, if appropriate, chromatographic purification of the crude products. Some of the intermediates and end products are obtained in the form of colourless or slightly brownish viscous oils which are purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

However, if the synthesis yields mixtures of isomers, a separation is generally not necessarily required since in some cases the individual isomers can be interconverted during work-up for use or during application (e.g. under the action of light, acids or bases). Such conversions may also take place after use, e.g. in the treatment of plants in the treated plant, or in the pest to be controlled.

In a preferred embodiment, the compounds I are present in form of a mixture of compounds I.A and I.B, wherein compound I.A with S-configuration in the isoxazoline ring is present in an amount of more than 50% by weight, in particular of at least 70% by weight, more particularly of at least 85% by weight, specifically of at least 90% by weight, based on the total weight of compounds I.A and I.B.

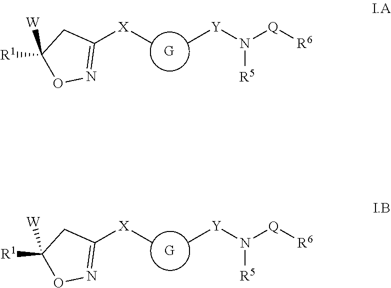

In one particularly preferred embodiment of the invention, the method for protecting growing plants from attack or infestation by invertebrate pests comprises the step of contacting the plant, parts of it, its propagation material, the pests, their food supply, habitat or breeding grounds a pesticidally effective amount of a compound of formula I.A.

Compounds of formula IA, and I.B, resp., can be obtained in enantiopure form by known separation methods, preferably by chiral chromatography. This is preferably applied to intermediate compounds of formula IA and IIB, or to compounds of formula I.

The organic moieties groups mentioned in the above definitions of the variables are—like the term halogen—collective terms for individual listings of the individual group members. The prefix Cn-Cm indicates in each case the possible number of carbon atoms in the group.

The term “partially or fully substituted” by a radical means that in general the group is substituted with same or different radicals.

The term “halogen” denotes in each case fluorine, bromine, chlorine, or iodine, in particular fluorine, chlorine, or bromine.

The term “alkyl” as used herein and in the alkyl moieties of alkylamino, alkylcarbonyl, alkylthio, alkylsulfinyl, alkylsulfonyl and alkoxyalkyl denotes in each case a straight-chain or branched alkyl group having usually from 1 to 10 carbon atoms, frequently from 1 to 6 carbon atoms, preferably 1 to 4 carbon atoms, more preferably from 1 to 3 carbon atoms. Examples of an alkyl group are methyl (Me), ethyl (Et), n-propyl (n-Pr), iso-propyl, n-butyl, 2-butyl, iso-butyl, tert-butyl, n-pentyl, 1-me¬thylbutyl, 2 methylbutyl, 3 methylbutyl, 2,2-di¬methylpropyl, 1 ethylpropyl, n-hexyl, 1,1-dimethyl-propyl, 1,2-dimethylpropyl, 1-methylpentyl, 2-methylpentyl, 3-methylpentyl, 4-methylpentyl, 1,1-dimethyl¬butyl, 1,2-dimethylbutyl, 1,3-dimethyl¬butyl, 2,2-dimethylbutyl, 2,3-dimethylbutyl, 3,3-dimethyl¬butyl, 1-ethylbutyl, 2-ethylbutyl, 1,1,2-trimethylpropyl, 1,2,2-trimethylpropyl, 1-ethyl-1-methyl¬propyl, and 1-ethyl-2-methylpropyl.

The term “haloalkyl” as used herein and in the haloalkyl moieties of haloalkylcarbonyl, haloalkoxycarbonyl, haloalkylthio, haloalkylsulfonyl, haloalkylsulfinyl, haloalkoxy and haloalkoxyalkyl, denotes in each case a straight-chain or branched alkyl group having usually from 1 to 10 carbon atoms, frequently from 1 to 6 carbon atoms, preferably from 1 to 4 carbon atoms, wherein the hydrogen atoms of this group are partially or totally replaced with halogen atoms. Preferred haloalkyl moieties are selected from C₁-C₄-halo¬alkyl, more preferably from C₁-C₃-haloalkyl or C₁-C₂-haloalkyl, in particular from C₁-C₂-fluoroalkyl such as fluoromethyl, difluoromethyl, trifluoromethyl, 1-fluoroethyl, 2-fluoroethyl, 2,2 difluoroethyl, 2,2,2-trifluoroethyl, pentafluoroethyl, and the like.

The term “alkoxy” as used herein denotes in each case a straight-chain or branched alkyl group which is bonded via an oxygen atom and has usually from 1 to 10 carbon atoms, frequently from 1 to 6 carbon atoms, preferably 1 to 4 carbon atoms. Examples of an alkoxy group are methoxy, ethoxy, n-propoxy, iso-prop¬oxy, n-butyloxy, 2-butyloxy, iso-butyloxy, tert.-butyloxy, and the like.

The term “alkoxyalkyl” as used herein refers to alkyl usually comprising 1 to 10, frequently 1 to 4, preferably 1 to 2 carbon atoms, wherein 1 carbon atom carries an alkoxy radical usually comprising 1 to 4, preferably 1 or 2 carbon atoms as defined above. Examples are CH₂OCH₃, CH₂—OC₂H₅, 2-(methoxy)ethyl, and 2-(ethoxy)ethyl.

The term “haloalkoxy” as used herein denotes in each case a straight-chain or branched alkoxy group having from 1 to 10 carbon atoms, frequently from 1 to 6 carbon atoms, preferably 1 to 4 carbon atoms, wherein the hydrogen atoms of this group are partially or totally replaced with halogen atoms, in particular fluorine atoms. Preferred haloalkoxy moieties include C₁-C₄-haloalkoxy, in particular C₁-C₂-fluoroalkoxy, such as fluoromethoxy, difluoromethoxy, trifluoromethoxy, 1 fluoroethoxy, 2-fluoroethoxy, 2,2 difluoroethoxy, 2,2,2-trifluoroethoxy, 2-chloro-2-fluoroethoxy, 2-chloro-2,2-difluoro-ethoxy, 2,2dichloro-2-fluorethoxy, 2,2,2-trichloroethoxy, penta¬fluoroethoxy and the like.

The term “alkylthio “(alkylsulfanyl: alkyl-S—)” as used herein refers to a straight-chain or branched saturated alkyl group having 1 to 10 carbon atoms, preferably 1 to 4 carbon atoms (═C₁-C₄-alkylthio), more preferably 1 to 3 carbon atoms, which is attached via a sulfur atom.

The term “haloalkylthio” as used herein refers to an alkylthio group as mentioned above wherein the hydrogen atoms are partially or fully substituted by fluorine, chlorine, bromine and/or iodine.

The term “alkylsulfinyl” (alkylsulfoxyl: C₁-C₆-alkyl-S(O)—), as used herein refers to a straight-chain or branched saturated alkyl group (as mentioned above) having 1 to 10 carbon atoms, preferably 1 to 4 carbon atoms (═C₁-C₄-alkylsulfinyl), more preferably 1 to 3 carbon atoms bonded through the sulfur atom of the sulfinyl group at any position in the alkyl group.

The term “haloalkylsulfinyl” as used herein refers to an alkylsulfinyl group as mentioned above wherein the hydrogen atoms are partially or fully substituted by fluorine, chlorine, bromine and/or iodine.

The term “alkylsulfonyl” (alkyl-S(O)₂—) as used herein refers to a straight-chain or branched saturated alkyl group having 1 to 10 carbon atoms, preferably 1 to 4 carbon atoms (═C₁-C₄-alkylsulfonyl), preferably 1 to 3 carbon atoms, which is bonded via the sulfur atom of the sulfonyl group at any position in the alkyl group.

The term “haloalkylsulfonyl” as used herein refers to an alkylsulfonyl group as mentioned above wherein the hydrogen atoms are partially or fully substituted by fluorine, chlorine, bromine and/or iodine.

The term “alkylcarbonyl” refers to an alkyl group as defined above, which is bonded via the car-bon atom of a carbonyl group (C═O) to the remainder of the molecule.

The term “haloalkylcarbonyl” refers to an alkylcarbonyl group as mentioned above, wherein the hydrogen atoms are partially or fully substituted by fluorine, chlorine, bromine and/or iodine.

The term “alkoxycarbonyl” refers to an alkylcarbonyl group as defined above, which is bonded via an oxygen atom to the remainder of the molecule.

The term “haloalkoxycarbonyl” refers to an alkoxycarbonyl group as mentioned above, wherein the hydrogen atoms are partially or fully substituted by fluorine, chlorine, bromine and/or iodine.

The term “alkenyl” as used herein denotes in each case a singly unsaturated hydrocarbon radical having usually 2 to 10, frequently 2 to 6, preferably 2 to 4 carbon atoms, e.g. vinyl, allyl (2-propen-1-yl), 1-propen-1-yl, 2 propen-2-yl, methallyl (2-methylprop-2-en-1-yl), 2-buten-1-yl, 3-buten-1-yl, 2-penten-1-yl, 3-penten-1-yl, 4-penten-1-yl, 1-methylbut-2-en-1-yl, 2-ethylprop-2-en-1-yl and the like.

The term “haloalkenyl” as used herein refers to an alkenyl group as defined above, wherein the hydrogen atoms are partially or totally replaced with halogen atoms.

The term “alkynyl” as used herein denotes in each case a singly unsaturated hydrocarbon radi-cal having usually 2 to 10, frequently 2 to 6, preferably 2 to 4 carbon atoms, e.g. ethynyl, propar-gyl (2-propyn-1-yl), 1-propyn-1-yl, 1-methylprop-2-yn-1-yl), 2-butyn-1-yl, 3-butyn-1-yl, 1-pentyn-1-yl, 3-pentyn-1-yl, 4-pentyn-1-yl, 1-methylbut-2-yn-1-yl, 1-ethylprop-2-yn-1-yl and the like.

The term “haloalkynyl” as used herein refers to an alkynyl group as defined above, wherein the hydrogen atoms are partially or totally replaced with halogen atoms.

The term “cycloalkyl” as used herein and in the cycloalkyl moieties of cycloalkoxy and cyclo-alkylthio denotes in each case a monocyclic cycloaliphatic radical having usually from 3 to 10 or from 3 to 6 carbon atoms, such as cyclopropyl (cC₃H₅), cyclobutyl (cC₄H₇), cyclopentyl (cC₅H₉), cyclohexyl (cC₆H₁₁), cycloheptyl, cyclooctyl, cyclononyl and cyclodecyl or cyclopropyl, cyclobutyl, cyclopentyl and cyclohexyl.

The term “halocycloalkyl” as used herein and in the halocycloalkyl moieties of halocycloalkoxy and halocycloalkylthio denotes in each case a monocyclic cycloaliphatic radical having usually from 3 to 10 C atoms or 3 to 6 C atoms, wherein at least one, e.g. 1, 2, 3, 4 or 5 of the hydrogen atoms, are replaced by halogen, in particular by fluorine or chlorine. Examples are 1- and 2-flu-orocyclopropyl, 1,2-, 2,2- and 2,3-difluorocyclopropyl, 1,2,2-trifluorocyclopropyl, 2,2,3,3-tetrafluorocyclpropyl, 1- and 2-chlorocyclopropyl, 1,2-, 2,2- and 2,3-dichlorocyclopropyl, 1,2,2-trichlorocyclopropyl, 2,2,3,3-tetrachlorocyclpropyl, 1-,2- and 3-fluorocyclopentyl, 1,2-, 2,2-, 2,3-, 3,3-, 3,4-, 2,5-difluorocyclopentyl, 1-,2- and 3-chlorocyclopentyl, 1,2-, 2,2-, 2,3-, 3,3-, 3,4-, 2,5-dichlorocyclopentyl and the like.

The term “halocycloalkenyl” as used herein and in the halocycloalkenyl moieties of halocycloalkenyloxy and halocycloalkenylthio denotes in each case a monocyclic singly unsaturated non-aromatic radical having usually from 3 to 10, e.g. 3 or 4 or from 5 to 10 carbon atoms, preferably from 3- to 8 carbon atoms, wherein at least one, e.g. 1, 2, 3, 4 or 5 of the hydrogen atoms, are replaced by halogen, in particular by fluorine or chlorine. Examples are 3,3-difluorocyclopropen-1-yl and 3,3-dichlorocyclopropen-1-yl.

The term “cycloalkenylalkyl” refers to a cycloalkenyl group as defined above which is bonded via an alkyl group, such as a C₁-C₅-alkyl group or a C₁-C₄-alkyl group, in particular a methyl group (=cycloalkenylmethyl), to the remainder of the molecule.

The term “carbocycle” or “carbocyclyl” includes in general a 3- to 12-membered, preferably a 3- to 8-membered or a 5- to 8-membered, more preferably a 5- or 6-membered monocyclic, non-aromatic ring comprising 3 to 12, preferably 3 to 8 or 5 to 8, more preferably 5 or 6 carbon atoms. Preferably, the term “carbocycle” covers cycloalkyl and cycloalkenyl groups as defined above.

The term “heterocycle” or “heterocyclyl” includes in general 3- to 12-membered, preferably 3- to 6-membered, in particular 6-membered monocyclic heterocyclic non-aromatic radicals. The heterocyclic non-aromatic radicals usually comprise 1, 2, 3, 4 or 5, preferably 1, 2 or 3 hetero¬atoms selected from N, O and S as ring members, wherein S-atoms as ring members may be present as S, SO or SO₂. Examples of 5- or 6-membered heterocyclic radicals comprise satu¬rated or unsaturated, non-aromatic heterocyclic rings, such as oxiranyl, oxetanyl, thietanyl, thietanyl-S-oxid (S-oxothietanyl), thietanyl-S-dioxid (S-dioxothiethanyl), pyrrolidinyl, pyrrolinyl, pyrazolinyl, tetrahydrofuranyl, dihydrofuranyl, 1,3-dioxolanyl, thiolanyl, S-oxothiolanyl, S-dioxo¬thiolanyl, dihydrothienyl, S-oxodihydrothienyl, S-dioxodihydro¬thienyl, oxazolidinyl, oxazolinyl, thiazolinyl, oxathiolanyl, piperidinyl, piperazinyl, pyranyl, dihydropyranyl, tetrahydropyranyl, 1,3- and 1,4-dioxanyl, thiopyranyl, S. oxothiopyranyl, S-dioxothiopyranyl, dihydrothio¬pyranyl, S-oxo¬dihydrothiopyranyl, S-dioxodihydrothiopyranyl, tetrahydrothiopyranyl, S-oxotetra¬hydrothiopyranyl, S-dioxotetrahydrothiopyranyl, morpholinyl, thiomorpholinyl, S-oxothio¬morpho¬linyl, S-dioxothiomorpholinyl, thiazinyl and the like. Examples for heterocyclic ring also comprising 1 or 2 carbonyl groups as ring members comprise pyrrolidin-2-onyl, pyrrolidin-2,5-dionyl, imidazolidin-2-onyl, oxazolidin-2-onyl, thiazolidin-2-only, and the like.

The term “hetaryl” includes monocyclic 5- or 6-membered heteroaromatic radicals comprising as ring members 1, 2, 3 or 4 heteroatoms selected from N, O and S. N- or S-containing hetaryl groups may be present as positively charged onium, and form together with a neighbouring atom a mesoionic entity. Examples of 5- or 6 membered heteroaromatic radicals include pyridyl, i.e. 2-, 3-, or 4 pyridyl, pyrimidinyl, i.e. 2, 4- or 5-pyrimidinyl, pyrazinyl, pyridazinyl, i.e. 3- or 4 pyridazinyl, thienyl, i.e. 2- or 3-thienyl, furyl, i.e. 2- or 3-furyl, pyrrolyl, i.e. 2- or 3 pyrrolyl, oxazolyl, i.e. 2-, 3- or 5-oxazolyl, isoxazolyl, i.e. 3-, 4- or 5-isoxazolyl, thiazolyl, i.e. 2-, 3- or 5-thiazolyl, isothiazolyl, i.e. 3-, 4- or 5 isothiazolyl, pyrazolyl, i.e. 1-, 3-, 4- or 5-pyrazolyl, i.e. 1-, 2-, 4- or 5-imidazolyl, oxadiazolyl, e.g. 2- or 5 [1,3,4]oxadiazolyl, 4- or 5-(1,2,3-oxa¬diazol)yl, 3- or 5-(1,2,4-oxadiazol)yl, 2- or 5 (1,3,4-thiadiazol)yl, thiadiazolyl, e.g. 2- or 5-(1,3,4-thia¬diazol)yl, 4- or 5 (1,2,3 thiadiazol)yl, 3- or 5-(1,2,4-thiadiazol)yl, triazolyl, e.g. 1H-, 2H- or 3H 1,2,3 triazol-4-yl, 2H-triazol-3-yl, 1H-, 2H-, or 4H-1,2,4-triazolyl and tetrazolyl, i.e. 1H- or 2H tetrazolyl. The term “hetaryl” also includes bicyclic 8 to 10-membered heteroaromatic radicals comprising as ring members 1, 2 or 3 heteroatoms selected from N, O and S, wherein a 5- or 6-membered heteroaromatic ring is fused to a phenyl ring or to a 5- or 6-membered heteroaromatic radical.

Examples of a 5- or 6-membered heteroaromatic ring fused to a phenyl ring or to a 5- or 6-membered heteroaromatic radical include benzofuranyl, benzo¬thienyl, indolyl, ind¬azolyl, benzimidazolyl, benzoxathiazolyl, benzoxadiazolyl, benzothiadiazolyl, benzoxazinyl, chinolinyl, isochinolinyl, purinyl, 1,8-naphthyridyl, pteridyl, pyrido[3,2 d]pyrimidyl or pyridoimidazolyl and the like. These fused hetaryl radicals may be bonded to the remainder of the molecule via any ring atom of 5- or 6-membered heteroaromatic ring or via a carbon atom of the fused phenyl moiety.

The terms “heterocyclylalkyl” and “hetarylalkyl” refer to heterocyclyl or hetaryl, respectively, as defined above which are bonded via a C₁-C₅-alkyl group or a C₁-C₄-alkyl group, in particular a methyl group (=heterocyclylmethyl or hetarylmethyl, respectively), to the remainder of the molecule.

The term “arylalkyl” and “phenylalkyl” refer to aryl as defined above and phenyl, respectively, which are bonded via C₁-C₅-alkyl group or a C₁-C₄-alkyl group, in particular a methyl group (=arylmethyl or phenylmethyl), to the remainder of the molecule, examples including benzyl, 1-phenylethyl, 2-phenylethyl, 2-phenoxyethyl etc.

The terms “alkylene”, “cycloalkylene”, “heterocycloalkylene”, “alkenylene”, “cycloalkenylene”, “heterocycloalkenylene” and “alkynylene” refer to alkyl, cycloalkyl, heterocycloalkyl, alkenyl, cycloalkenyl, heterocycloalkenyl and alkynyl as defined above, respectively, which are bonded to the remainder of the molecule, via two atoms, preferably via two carbon atoms, of the respective group, so that they represent a linker between two moieties of the molecule.

In a particular embodiment, the variables of the compounds of the formula I have the following meanings, these meanings, both on their own and in combination with one another, being particular embodiments of the compounds of the formula I.

Embodiments and preferred compounds of the invention for use in pesticidal methods and for insecticidal application purposes are outlined in the following paragraphs.

With respect to the variables, the particularly preferred embodiments of the intermediates correspond to those of the compounds of the formula I.

In one embodiment W is a group WA,

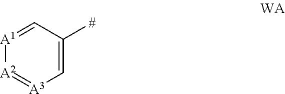

wherein
A¹,A²,A³ are N or CR², provided that at least two are CR²;
R² are independently from each other preferably H, halogen, halomethyl, halomethoxy, or halomethyl-S(O)ₘ.

In another embodiment W is phenyl, which is substituted with one to three groups halogen, halomethyl, halomethoxy, and/or halomethyl-S(O)ₘ.

W is preferably a group WP:

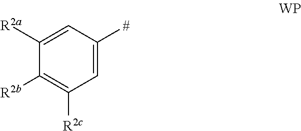

wherein R^{2a}, R^{2b}, and R^{2c} are a group R², preferably selected from halogen, halomethyl, halomethoxy, and halomethyl-S(O)ₘ.

Particularly preferred is each one of the following combinations of R^{2a}, R^{2b} and R^{2c} wherein each line of Table W denotes a substitution pattern of the phenyl ring (“WP”) bearing the R^{2a}, R^{2b} and R^{2c} moieties.

TABLE W

| No. | R^{2a} | R^{2b} | R^{2c} |
|---|---|---|---|
| W-1 | F | F | H |
| W-2 | F | H | F |
| W-3 | F | F | F |
| W-4 | F | Cl | F |
| W-5 | F | Br | F |
| W-6 | F | H | Cl |
| W-7 | F | H | Br |
| W-8 | Cl | F | H |
| W-9 | Cl | H | Cl |
| W-10 | Cl | Cl | Cl |
| W-11 | Cl | F | Cl |
| W-12 | Cl | Br | Cl |
| W-13 | Cl | H | Br |
| W-14 | Br | F | H |
| W-15 | Br | H | Br |
| W-16 | Br | F | Br |
| W-17 | Br | Cl | Br |
| W-18 | CF₃ | H | H |
| W-19 | CF₃ | H | F |
| W-20 | CF₃ | H | Cl |
| W-21 | CF₃ | H | Br |
| W-22 | CF₃ | H | CF₃ |
| W-23 | CF₃ | F | F |
| W-24 | CF₃ | F | Cl |
| W-25 | CF₃ | Cl | Cl |
| W-26 | CF₃ | F | H |
| W-27 | OCF₃ | H | F |
| W-28 | OCF₃ | H | Cl |
| W-29 | OCF₃ | F | H |
| W-30 | OCF₃ | H | CF3 |
| W-31 | OCF₃ | H | H |
| W-32 | Cl | OCF₃ | H |
| W-33 | Cl | Cl | H |
| W-34 | SCF₃ | F | H |

Groups W-8, W-9, W-11, and W-33 are more preferred patterns in formula I compounds. W-8 and W-11 are particularly preferred.

In another embodiment group W is selected from W-8, W-9, W-11, W-33, and W-34.

Another embodiment of W is a 6-membered hetaryl, such as thiophene, pyrazole, imidazole, or pyridine, which rings are substituted with 1 or 2 groups R^{2a} or C₁-C₂-alkoxy-C₁-C₂-alkyl.

R¹ is preferably methyl, substituted with 1 to 3 atoms selected from C₁ and F, such as CF₃, CHF₂, CClF₂, CCl₂F, CCl₃, CHCl₂, or CHFCl.

X is preferably NR³.

R³ is preferably H, C₁-C₆-alkoxycarbonyl, or C₁-C₆-alkyl, which alkyl is unsubstituted or substituted with CN, C₃-C₆-cycloalkyl, and/or C₁-C₆-alkoxy. R³ being H, C₂H₅, or CH₂OCH₃ is particularly preferred.

In another embodiment R³ is OH, C₁-C₄-alkoxy, or C₁-C₄-alkoxycarbonyl.

In another embodiment X is O.

In one embodiment G is a group GA:

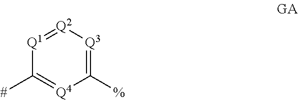

wherein
Q¹, Q², Q³, Q⁴ are N or CR⁴; provided that at least three are CR⁴;
R⁴ are independently from each other preferably H, halogen, CN, C₁-C₄-alkyl, C₁-C₄-cycloalkyl, C₁-C₄-haloalkyl, C₁-C₄-halocycloalkyl, C₁-C₃-alkoxy, C₁-C₃-haloalkoxy, S(O)ₘ—C₁-C₃-alkyl, S(O)ₘ-C₁-C₃-haloalkyl.

G is preferably a group G1, G2, G3, or G4:

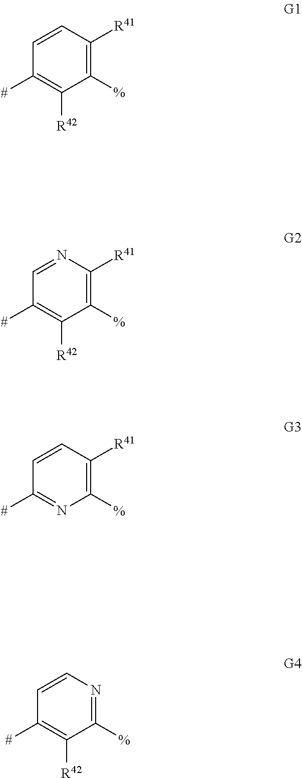

wherein #is the bond to X, % is the bond to Y, and R⁴¹ and R⁴² are H or a group R², preferably H, or halogen, CN, halomethyl, halomethoxy. In a particularly preferred embodiment R⁴¹ is CN, and halogen such as Cl, and R⁴² is H.

In a preferred embodiment G is G1 and R⁴² is H.

In another embodiment G is selected from G1 and G4, wherein R⁴² is H.

In a preferred embodiment Y is C(R^{4a})(R^{4b}), preferably CH₂.

In a preferred embodiment Q is a pyrazole, imidazole, or triazole; preferably selected from 1-pyrazole, 1-imidazole, and 1,2,4-triazole, which are unsubstituted or substituted with CN, C₁-C₄-alkyl, C₁-C₄-haloalkyl, C₁-C₄-haloalkoxy, S(O)ₘ—C₁-C₄-alkyl, S(O)ₘ—C₁-C₄-haloalkyl, NR¹⁵C(═O)R²⁶, or C(═O)NR^{25a}R^{25b}, wherein R^{25a} and R^{25b} are preferably H, C₁-C₄-alkyl, C₁-C₄-haloalkyl, and C₃-C₆-cycloalkyl.

In a preferred embodiment Q is C(═O).

A preferred embodiment are compounds of formula I, wherein
R¹ is C₁-C₂-haloalkyl, preferably halomethyl;
W is phenyl, which is partially or fully substituted with R²;
   R² is halogen, CN, OH, C₁-C₄-alkoxy, OR²¹, C₁-C₄-haloalkyl, C₁-C₃-alkoxy, C₁-C₃-haloalkoxy, C₁-C₃-alkyl-S(O)ₘ, C₁-C₃-haloalkyl-S(O)ₘ, C₁-C₃-alkoxy-C₁-C₄-alkyl;
X is NR³ or O;
R³ is H, C₁-C₄-alkyl, C₁-C₄-alkoxy-C₁-C₄-alkyl, C₁-C₄-alkoxycarbonyl, C₃-C₆-cycloalkyl-C₁-C₄-alkyl, C₂-C₄-alkenyl, C₂-C₄-alkynyl,
G is phenyl, which is unsubstituted, partially, or fully substituted with R⁴; preferably a group G1, wherein R⁴¹ and R⁴² are H, halogen, C₁-C₄-alkyl, C₁-C₄-haloalkyl, C₁-C₄-alkoxy, C₁-C₄-haloalkoxy;
R⁴ is H, halogen C₁-C₄-alkyl, C₁-C₄-haloalkyl, C₁-C₄-alkoxy, C₁-C₄-haloalkoxy; or R⁴ and R^{4a}/R^{4b} together form a 5-, or 6-membered saturated or unsaturated carbo- or heterocycle, which heterocycle may contain 1 or 2 N as ring members;
Y is a direct bond or CR^{4a}R^{4b};
Q is C(═O) or SO₂;
R⁵ H,
R⁶ C₁-C₄-alkyl, C₁-C₄-haloalkyl, C₃-C₆-cycloalkyl, C₃-C₆-cycloalkyl-C₁-C₄-alkyl, C₃-C₆-cycloalkenyl, C₁-C₄-alkoxy-C₁-C₄-alkyl, C₁-C₄-alkyl-S(O)ₘ—C₁-C₄-alkyl, NR^{H}C(═O)—C₁-C₄-alkyl, NR^{H}C(═O)—C₃-C₆-cycloalkyl, CR^{H}N═OR²⁴, CR^{H}N═OR²⁴, 4-, 5-, or 6-membered saturated or partially or fully unsaturated heterocycle containing 1, 2 or 3 heteroatoms selected from O, N, S as ring members, wherein S may be oxidized,
   wherein R^{H} is H, CN or C₁-C₄-alkyl,
   R⁶ being unsubstituted or substituted with CN, C₁-C₄-alkoxy, C₁-C₄-haloalkoxy, S(O)ₘ—C₁-C₄-alkyl, S(O)ₘ—C₁-C₄-haloalkyl, C₃-C₆-cycloalkyl which is unsubstituted or substituted with 1 or 2 halogen and/or CN.

A preferred embodiment are compounds of formula I which correspond to formula I.A as depicted below, wherein the variables are defined and preferred as above.

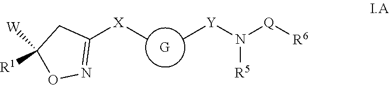

Another preferred embodiment are compounds of formula I which correspond to formula I′ as depicted below, wherein the variables are defined and preferred as above.

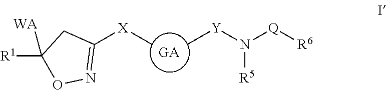

Another preferred embodiment are compounds of formula I which correspond to formula I.1 as depicted below, wherein the variables are defined and preferred as above.

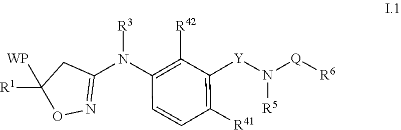

In particular with a view to their use, preference is given to the compounds of formula I com-piled in the tables below, which compounds correspond to formula I.1 wherein R¹ is CF₃ and R⁵ and R⁴² denote H. Each of the groups mentioned for a substituent in the tables is furthermore per se, independently of the combination in which it is mentioned, a particularly preferred aspect of the substituent in question.

Table 1: Compounds of formula I.1 in which Q is C═O, R⁶ is CH₃, and the other variables for a compound correspond in each case to one row of Table A

Table 2: Compounds of formula I.1 in which Q is C═O, R⁶ is C₂H₅, and the other variables for a compound correspond in each case to one row of Table A

Table 3: Compounds of formula I.1 in which Q is C═O, R⁶ is CH₂CH₂CH₃, and the other variables for a compound correspond in each case to one row of Table A

Table 4: Compounds of formula I.1 in which Q is CH(CH₃)₂, and the other variables for a compound correspond in each case to one row of Table A

Table 5: Compounds of formula I.1 in which Q is C═O, R⁶ is cC₃H₅, and the other variables for a compound correspond in each case to one row of Table A

Table 6: Compounds of formula I.1 in which Q is C═O, R⁶ is (1-CN-)cC₃H₄, and the other variables for a compound correspond in each case to one row of Table A

Table 7: Compounds of formula I.1 in which Q is C═O, R⁶ is CH₂OCH₃, and the other variables for a compound correspond in each case to one row of Table A

Table 8: Compounds of formula I.1 in which Q is C═O, R⁶ is CH₂CH₂OCH₃, and the other variables for a compound correspond in each case to one row of Table A

Table 9: Compounds of formula I.1 in which Q is SO₂, R⁶ is CH₃, and the other variables for a compound correspond in each case to one row of Table A

Table 10: Compounds of formula I.1 in which Q is SO₂, R⁶ is C₂H₅, and the other variables for a compound correspond in each case to one row of Table A

Table 11: Compounds of formula I.1 in which Q is SO₂, R⁶ is CH₂CH₂CH₃, and the other variables for a compound correspond in each case to one row of Table A

Table 12: Compounds of formula I.1 in which Q is SO₂, R⁶ is CH(CH₃)₂, and the other variables for a compound correspond in each case to one row of Table A

Table 13: Compounds of formula I.1 in which Q is SO₂, R⁶ is cC₃H₅, and the other variables for a compound correspond in each case to one row of Table A

Table 14: Compounds of formula I.1 in which Q is SO₂, R⁶ is (1-CN-)cC₃H₄, and the other variables for a compound correspond in each case to one row of Table A

Table 15: Compounds of formula I.1 in which Q is SO₂, R⁶ is CH₂OCH₃, and the other variables for a compound correspond in each case to one row of Table A

Table 16: Compounds of formula I.1 in which Q is SO₂, R⁶ is CH₂CH₂OCH₃, and the other variables for a compound correspond in each case to one row of Table A

TABLE A

| No. | WP | R³ | R⁴¹ |
|---|---|---|---|
| A-1 | W-8 | H | Cl |
| A-2 | W-9 | H | Cl |
| A-3 | W-10 | H | Cl |
| A-4 | W-11 | H | Cl |
| A-5 | W-18 | H | Cl |
| A-6 | W-20 | H | Cl |
| A-7 | W-26 | H | Cl |
| A-8 | W-29 | H | Cl |
| A-9 | W-33 | H | Cl |
| A-10 | W-8 | CH₃ | Cl |
| A-11 | W-9 | CH₃ | Cl |
| A-12 | W-10 | CH₃ | Cl |
| A-13 | W-11 | CH₃ | Cl |
| A-14 | W-18 | CH₃ | Cl |
| A-15 | W-20 | CH₃ | Cl |
| A-16 | W-26 | CH₃ | Cl |
| A-17 | W-29 | CH₃ | Cl |
| A-18 | W-33 | CH₃ | Cl |
| A-19 | W-8 | CH₂CH₃ | Cl |
| A-20 | W-9 | CH₂CH₃ | Cl |
| A-21 | W-10 | CH₂CH₃ | Cl |
| A-22 | W-11 | CH₂CH₃ | Cl |
| A-23 | W-18 | CH₂CH₃ | Cl |
| A-24 | W-20 | CH₂CH₃ | Cl |
| A-25 | W-26 | CH₂CH₃ | Cl |
| A-26 | W-29 | CH₂CH₃ | Cl |
| A-27 | W-33 | CH₂CH₃ | Cl |
| A-28 | W-8 | CH₂OCH₃ | Cl |
| A-29 | W-9 | CH₂OCH₃ | Cl |
| A-30 | W-10 | CH₂OCH₃ | Cl |
| A-31 | W-11 | CH₂OCH₃ | Cl |
| A-32 | W-18 | CH₂OCH₃ | Cl |
| A-33 | W-20 | CH₂OCH₃ | Cl |
| A-34 | W-26 | CH₂OCH₃ | Cl |
| A-35 | W-29 | CH₂OCH₃ | Cl |
| A-36 | W-33 | CH₂OCH₃ | Cl |
| A-37 | W-8 | H | F |
| A-38 | W-9 | H | F |
| A-39 | W-10 | H | F |
| A-40 | W-11 | H | F |
| A-41 | W-18 | H | F |
| A-42 | W-20 | H | F |
| A-43 | W-26 | H | F |
| A-44 | W-29 | H | F |
| A-45 | W-33 | H | F |
| A-46 | W-8 | CH₃ | F |
| A-47 | W-9 | CH₃ | F |
| A-48 | W-10 | CH₃ | F |
| A-49 | W-11 | CH₃ | F |
| A-50 | W-18 | CH₃ | F |
| A-51 | W-20 | CH₃ | F |
| A-52 | W-26 | CH₃ | F |
| A-53 | W-29 | CH₃ | F |
| A-54 | W-33 | CH₃ | F |
| A-55 | W-8 | CH₂CH₃ | F |
| A-56 | W-9 | CH₂CH₃ | F |
| A-57 | W-10 | CH₂CH₃ | F |
| A-58 | W-11 | CH₂CH₃ | F |
| A-59 | W-18 | CH₂CH₃ | F |
| A-60 | W-20 | CH₂CH₃ | F |
| A-61 | W-26 | CH₂CH₃ | F |
| A-62 | W-29 | CH₂CH₃ | F |
| A-63 | W-33 | CH₂CH₃ | F |
| A-64 | W-8 | CH₂OCH₃ | F |
| A-65 | W-9 | CH₂OCH₃ | F |
| A-66 | W-10 | CH₂OCH₃ | F |
| A-67 | W-11 | CH₂OCH₃ | F |
| A-68 | W-18 | CH₂OCH₃ | F |
| A-69 | W-20 | CH₂OCH₃ | F |
| A-70 | W-26 | CH₂OCH₃ | F |
| A-71 | W-29 | CH₂OCH₃ | F |
| A-72 | W-33 | CH₂OCH₃ | F |
| A-73 | W-8 | H | CH₃ |
| A-74 | W-9 | H | CH₃ |
| A-75 | W-10 | H | CH₃ |
| A-76 | W-11 | H | CH₃ |
| A-77 | W-18 | H | CH₃ |
| A-78 | W-20 | H | CH₃ |
| A-79 | W-26 | H | CH₃ |
| A-80 | W-29 | H | CH₃ |
| A-81 | W-33 | H | CH₃ |
| A-82 | W-8 | CH₃ | CH₃ |
| A-83 | W-9 | CH₃ | CH₃ |
| A-84 | W-10 | CH₃ | CH₃ |
| A-85 | W-11 | CH₃ | CH₃ |
| A-86 | W-18 | CH₃ | CH₃ |
| A-87 | W-20 | CH₃ | CH₃ |
| A-88 | W-26 | CH₃ | CH₃ |
| A-89 | W-29 | CH₃ | CH₃ |
| A-90 | W-33 | CH₃ | CH₃ |
| A-91 | W-8 | CH₂CH₃ | CH₃ |
| A-92 | W-9 | CH₂CH₃ | CH₃ |
| A-93 | W-10 | CH₂CH₃ | CH₃ |
| A-94 | W-11 | CH₂CH₃ | CH₃ |
| A-95 | W-18 | CH₂CH₃ | CH₃ |
| A-96 | W-20 | CH₂CH₃ | CH₃ |
| A-97 | W-26 | CH₂CH₃ | CH₃ |
| A-98 | W-29 | CH₂CH₃ | CH₃ |
| A-99 | W-33 | CH₂CH₃ | CH₃ |
| A-100 | W-8 | CH₂OCH₃ | CH₃ |
| A-101 | W-9 | CH₂OCH₃ | CH₃ |
| A-102 | W-10 | CH₂OCH₃ | CH₃ |
| A-103 | W-11 | CH₂OCH₃ | CH₃ |
| A-104 | W-18 | CH₂OCH₃ | CH₃ |
| A-105 | W-20 | CH₂OCH₃ | CH₃ |
| A-106 | W-26 | CH₂OCH₃ | CH₃ |
| A-107 | W-29 | CH₂OCH₃ | CH₃ |
| A-108 | W-33 | CH₂OCH₃ | CH₃ |
| A-109 | W-8 | H | OCF₃ |
| A-110 | W-9 | H | OCF₃ |
| A-111 | W-10 | H | OCF₃ |
| A-112 | W-11 | H | OCF₃ |
| A-113 | W-18 | H | OCF₃ |
| A-114 | W-20 | H | OCF₃ |
| A-115 | W-26 | H | OCF₃ |
| A-116 | W-29 | H | OCF₃ |
| A-117 | W-33 | H | OCF₃ |
| A-118 | W-8 | CH₃ | OCF₃ |
| A-119 | W-9 | CH₃ | OCF₃ |
| A-120 | W-10 | CH₃ | OCF₃ |
| A-121 | W-11 | CH₃ | OCF₃ |
| A-122 | W-18 | CH₃ | OCF₃ |
| A-123 | W-20 | CH₃ | OCF₃ |
| A-124 | W-26 | CH₃ | OCF₃ |
| A-125 | W-29 | CH₃ | OCF₃ |
| A-126 | W-33 | CH₃ | OCF₃ |
| A-127 | W-8 | CH₂CH₃ | OCF₃ |
| A-128 | W-9 | CH₂CH₃ | OCF₃ |
| A-129 | W-10 | CH₂CH₃ | OCF₃ |
| A-130 | W-11 | CH₂CH₃ | OCF₃ |
| A-131 | W-18 | CH₂CH₃ | OCF₃ |
| A-132 | W-20 | CH₂CH₃ | OCF₃ |
| A-133 | W-26 | CH₂CH₃ | OCF₃ |
| A-134 | W-29 | CH₂CH₃ | OCF₃ |
| A-135 | W-33 | CH₂CH₃ | OCF₃ |
| A-136 | W-8 | CH₂OCH₃ | OCF₃ |
| A-137 | W-9 | CH₂OCH₃ | OCF₃ |
| A-138 | W-10 | CH₂OCH₃ | OCF₃ |
| A-139 | W-11 | CH₂OCH₃ | OCF₃ |
| A-140 | W-18 | CH₂OCH₃ | OCF₃ |
| A-141 | W-20 | CH₂OCH₃ | OCF₃ |
| A-142 | W-26 | CH₂OCH₃ | OCF₃ |
| A-143 | W-29 | CH₂OCH₃ | OCF₃ |
| A-144 | W-33 | CH₂OCH₃ | OCF₃ |
| A-145 | W-8 | H | CN |
| A-146 | W-9 | H | CN |
| A-147 | W-10 | H | CN |
| A-148 | W-11 | H | CN |
| A-149 | W-18 | H | CN |
| A-150 | W-20 | H | CN |
| A-151 | W-26 | H | CN |
| A-152 | W-29 | H | CN |
| A-153 | W-33 | H | CN |
| A-154 | W-8 | CH₃ | CN |
| A-155 | W-9 | CH₃ | CN |
| A-156 | W-10 | CH₃ | CN |
| A-157 | W-11 | CH₃ | CN |
| A-158 | W-18 | CH₃ | CN |
| A-159 | W-20 | CH₃ | CN |
| A-160 | W-26 | CH₃ | CN |
| A-161 | W-29 | CH₃ | CN |
| A-162 | W-33 | CH₃ | CN |
| A-163 | W-8 | CH₂CH₃ | CN |
| A-164 | W-9 | CH₂CH₃ | CN |
| A-165 | W-10 | CH₂CH₃ | CN |
| A-166 | W-11 | CH₂CH₃ | CN |
| A-167 | W-18 | CH₂CH₃ | CN |
| A-168 | W-20 | CH₂CH₃ | CN |
| A-169 | W-26 | CH₂CH₃ | CN |
| A-170 | W-29 | CH₂CH₃ | CN |
| A-171 | W-33 | CH₂CH₃ | CN |
| A-172 | W-8 | CH₂OCH₃ | CN |
| A-173 | W-9 | CH₂OCH₃ | CN |
| A-174 | W-10 | CH₂OCH₃ | CN |
| A-175 | W-11 | CH₂OCH₃ | CN |
| A-176 | W-18 | CH₂OCH₃ | CN |
| A-177 | W-20 | CH₂OCH₃ | CN |
| A-178 | W-26 | CH₂OCH₃ | CN |
| A-179 | W-29 | CH₂OCH₃ | CN |
| A-180 | W-33 | CH₂OCH₃ | CN |

The term “compound(s) of the invention” refers to compound(s) of formula I, or “compound(s) I”, and includes their salts, tautomers, stereoisomers, and N-oxides.

The invention also relates to agrochemical compositions comprising an auxiliary and at least one compound 1.

An agrochemical composition comprises a pesticidally effective amount of a compound 1.

The compounds I can be converted into customary types of agro-chemical compositions, e.g. solutions, emulsions, suspensions, dusts, powders, pastes, granules, pressings, capsules, and mixtures thereof. Examples for composition types are suspensions (e.g. SO, OD, FS), emulsifiable concentrates (e.g. EC), emulsions (e.g. EW, EO, ES, ME), capsules (e.g. OS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials e.g. seeds (e.g. GF). These and further compositions types are defined in the “Catalogue of pesticide formulation types and international coding system”, Technical Monograph No. 2, 6th Ed. May 2008, CropLife International.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents. Suitable solid carriers or fillers are mineral earths.

Suitable surfactants are surface-active compounds, e.g. anionic, cationic, nonionic, and amphoteric surfactants, block polymers, polyelectrolytes. Such surfactants can be used as emulsifier, dispersant, solubilizer, wetter, penetration enhancer, protective colloid, or adjuvant.

Surfactants are listed in McCutcheon's, Vol. 1: Emulsifiers & Detergents, McCutcheon's Directories, Glen Rock, USA, 2008 (International or North American Ed.). Suitable anionic surfactants are alkali, alkaline earth, or ammonium salts of sulfonates, sulfates, phosphates, carboxylates. Suitable nonionic surfactants are alkoxylates, N-subsituted fatty acid amides, amine oxides, esters, sugar-based surfactants, polymeric surfactants. Suitable cationic surfactants are quaternary surfactants.

The agrochemical compositions generally comprise between 0.01 and 95%, preferably between 0.1 and 90%, and most preferably between 0.5 and 75%, by weight of active substance. The active substances are employed in a purity of from 90% to 100%, preferably from 95% to 100%.

Various types of oils, wetters, adjuvants, or fertilizer may be added to the active substances or the compositions comprising them as premix or, if appropriate not until immediately prior to use (tank mix). These agents can be admixed with the compositions according to the invention in a weight ratio of 1:100 to 100:1.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters of the ready-to-use spray liquor are applied per hectare of agricultural useful area.

The compounds I are suitable for use in protecting crops, plants, plant propagation materials, e.g. seeds, or soil or water, in which the plants are growing, from attack or infestation by animal pests. Therefore, the invention also relates to a plant protection method, which comprises contacting crops, plants, plant propagation materials, e.g. seeds, or soil or water, in which the plants are growing, to be protected from attack or infestation by animal pests, with a pesticidally effective amount of a compound I.

The compounds I are also suitable for use in combating or controlling animal pests. Therefore, the invention also relates to a method of combating or controlling animal pests, which comprises contacting the animal pests, their habitat, breeding ground, or food supply, or the crops, plants, plant propagation materials, e.g. seeds, or soil, or the area, material or environment in which the animal pests are growing or may grow, with a pesticidally effective amount of a compound I.

The compounds I are effective through both contact and ingestion to any and all developmental stages, such as egg, larva, pupa, and adult.

The compounds I can be applied as such or in form of compositions comprising them.

The application can be carried out both before and after the infestation of the crops, plants, plant propagation materials by the pests.

The term “contacting” includes both direct contact (applying the compounds/compositions directly on the animal pest or plant) and indirect contact (applying the compounds/compositions to the locus).

The term “animal pest” includes arthropods, gastropods, and nematodes. Preferred animal pests according to the invention are arthropods, preferably insects and arachnids, in particular insects.

The term “plant” includes cereals, e.g. durum and other wheat, rye, barley, triticale, oats, rice, or maize (fodder maize and sugar maize/sweet and field corn); beet, e.g. sugar beet, or fodder beet; fruits, e.g. pomes, stone fruits, or soft fruits, e.g. apples, pears, plums, peaches, nectarines, almonds, cherries, papayas, strawberries, raspberries, blackberries or gooseberries; leguminous plants, e.g. beans, lentils, peas, alfalfa, or soybeans; oil plants, e.g. rapeseed (oilseed rape), turnip rape, mustard, olives, sunflowers, coconut, cocoa beans, castor oil plants, oil palms, ground nuts, or soybeans; cucurbits, e.g. squashes, pumpkins, cucumber or melons; fiber plants, e.g. cotton, flax, hemp, or jute; citrus fruit, e.g. oranges, lemons, grapefruits or mandarins; vegetables, e.g. eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, e.g. avocados, cinnamon, or camphor; energy and raw material plants, e.g. corn, soybean, rapeseed, sugar cane or oil palm; tobacco; nuts, e.g. walnuts; pistachios; coffee; tea; bananas; vines; hop; sweet leaf (Stevia); natural rubber plants or ornamental and forestry plants, shrubs, broad-leaved trees or evergreens, eucalyptus; turf; lawn; grass. Preferred plants include potatoes, sugar beets, tobacco, wheat, rye, barley, oats, rice, corn, cotton, soybeans, rapeseed, legumes, sunflowers, coffee, or sugar cane; fruits; vines; ornamentals; or vegetables, e.g. cucumbers, tomatoes, beans or squashes.

The term “seed” embraces seeds and plant propagules including true seeds, seed pieces, suckers, corms, bulbs, fruit, tubers, grains, cuttings, cut shoots, and means preferably true seeds.

“Pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions e.g. desired pesticidal effect and duration, weather, target species, locus, mode of application.

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare.

The compounds I are also suitable for use against non-crop insect pests. For use against said non-crop pests, compounds I can be used as bait composition, gel, general insect spray, aerosol, as ultra-low volume application and bed net (impregnated or surface applied).

The term “non-crop insect pest” refers to pests, which are particularly relevant for non-crop tar-gets, e.g. ants, termites, wasps, flies, ticks, mosquitoes, bed bugs, crickets, or cockroaches, such as: Aedes aegypti, Musca domestica, Tribolium spp.; termites such as Reticulitermes flavipes, Coptotermes formosanus; roaches such as Blatella germanica, Periplaneta Americana; ants such as Solenopsis invicta, Linepithema humile, and Camponotus pennsylvanicus.

The bait can be a liquid, a solid or a semisolid preparation (e.g. a gel). For use in bait compositions, the typical content of active ingredient is from 0.001 wt % to 15 wt %, desirably from 0.001 wt % to 5 wt % of active compound.

The compounds I and its compositions can be used for protecting wooden materials such as trees, board fences, sleepers, frames, artistic artifacts, etc. and buildings, but also construction materials, furniture, leathers, fibers, vinyl articles, electric wires and cables etc. from ants, termites and/or wood or textile destroying beetles, and for controlling ants and termites from doing harm to crops or human beings (e.g. when the pests invade into houses and public facilities or nest in yards, orchards or parks).

Customary application rates in the protection of materials are, e.g., from 0.001 g to 2000 g or from 0.01 g to 1000 g of active compound per m² treated material, desirably from 0.1 g to 50 g per m².

Insecticidal compositions for use in the impregnation of materials typically contain from 0.001 to 95 wt %, preferably from 0.1 to 45 wt %, and more preferably from 1 to 25 wt % of at least one repellent and/or insecticide.

The compounds of the invention are especially suitable for efficiently combating animal pests e.g. arthropods, and nematodes including:
insects from the sub-order of Auchenorrhyncha, e.g. Amrasca biguttula, Empoasca spp., Nephotettix virescens, Sogatella furcifera, Mahanarva spp., Laodelphax striatellus, Nilaparvata lugens, Diaphorina citri;
Lepidoptera, e.g. Helicoverpa spp., Heliothis virescens, Lobesia botrana, Ostrinia nubilalis, Plu-tella xylostella, Pseudoplusia includens, Scirpophaga incertulas, Spodoptera spp., Trichoplusia ni, Tuta absoluta, Cnaphalocrocis medialis, Cydia pomonella, Chilo suppressalis, Anticarsia gemmatalis, Agrotis ipsilon, Chrysodeixis includens;
True bugs, e.g. Lygus spp., Stink bugs such as Euschistus spp., Halyomorpha halys, Nezara viridula, Piezodorus guildinii, Dichelops furcatus;
Thrips, e.g. Frankliniella spp., Thrips spp., Dichromothrips corbettii;
Aphids, e.g. Acyrthosiphon pisum, Aphis spp., Myzus persicae, Rhopalosiphum spp., Schizaphis graminum, Megoura viciae;
Whiteflies, e.g. Trialeurodes vaporariorum, Bemisia spp.;
Coleoptera, e.g. Phyllotreta spp., Melanotus spp., Meligethes aeneus, Leptinotarsa decimlineata, Ceutorhynchus spp., Diabrotica spp., Anthonomus grandis, Atomaria linearia, Agriotes spp., Epilachna spp.;
Flies, e.g. Delia spp., Ceratitis capitate, Bactrocera spp., Liriomyza spp.;
Mosquitoes (Diptera), e.g. Aedes aegypti, A. albopictus, A. vexans, Anastrepha ludens, Anopheles maculipennis, A. crucians, A. albimanus, A. gambiae, A. freeborni, A. leucosphyrus, A. minimus, A. quadrimaculatus;
Coccoidea, e.g. Aonidiella aurantia, Ferrisia virgate;
Anthropods of class Arachnida (Mites), e.g. Penthaleus major, Tetranychus spp.;
Nematodes, e.g. Heterodera glycines, Meloidogyne sp., Pratylenchus spp., Caenorhabditis elegans.

The compounds I are suitable for use in treating or protecting animals against infestation or infection by parasites. Therefore, the invention also relates to the use of a compound of the invention for the manufacture of a medicament for the treatment or protection of animals against infestation or infection by parasites. Furthermore, the invention relates to a method of treating or protecting animals against infestation and infection by parasites, which comprises orally, topically or parenterally administering or applying to the animals a parasiticidally effective amount of a compound I.

The invention also relates to the non-therapeutic use of compounds of the invention for treating or protecting animals against infestation and infection by parasites. Moreover, the invention relates to a non-therapeutic method of treating or protecting animals against infestation and infection by parasites, which comprises applying to a locus a parasiticidally effective amount of a compound I.

The compounds of the invention are further suitable for use in combating or controlling parasites in and on animals. Furthermore, the invention relates to a method of combating or controlling parasites in and on animals, which comprises contacting the parasites with a parasitically effective amount of a compound I.

The invention also relates to the non-therapeutic use of compounds I for controlling or combating parasites. Moreover, the invention relates to a non-therapeutic method of combating or con-trolling parasites, which comprises applying to a locus a parasiticidally effective amount of a compound I.

The compounds I can be effective through both contact (via soil, glass, wall, bed net, carpet, blankets, or animal parts) and ingestion (e.g. baits). Furthermore, the compounds I can be applied to any and all developmental stages.

The compounds I can be applied as such or in form of compositions comprising them.

The term “locus” means the habitat, food supply, breeding ground, area, material or environment in which a parasite is growing or may grow outside of the animal.

As used herein, the term “parasites” includes endo- and ectoparasites. In some embodiments of the invention, endoparasites can be preferred. In other embodiments, ectoparasites can be preferred. Infestations in warm-blooded animals and fish include lice, biting lice, ticks, nasal bots, keds, biting flies, muscoid flies, flies, myiasitic fly larvae, chiggers, gnats, mosquitoes and fleas.

The compounds of the invention are especially useful for combating the following parasites: Cimex lectularius, Rhipicephalus sanguineus, and Ctenocephalides felis.

As used herein, the term “animal” includes warm-blooded animals (including humans) and fish.

Preferred are mammals, such as cattle, sheep, swine, camels, deer, horses, pigs, poultry, rabbits, goats, dogs and cats, water buffalo, donkeys, fallow deer and reindeer, and also in furbearing animals such as mink, chinchilla and raccoon, birds such as hens, geese, turkeys and ducks and fish such as fresh- and salt-water fish such as trout, carp and eels. Particularly preferred are domestic animals, such as dogs or cats.

The compounds I may be applied in total amounts of 0.5 mg/kg to 100 mg/kg per day, preferably 1 mg/kg to 50 mg/kg per day.

For oral administration to warm-blooded animals, the compounds I may be formulated as animal feeds, animal feed premixes, animal feed concentrates, pills, solutions, pastes, suspensions, drenches, gels, tablets, boluses and capsules. For oral administration, the dosage form chosen should provide the animal with 0.01 mg/kg to 100 mg/kg of animal body weight per day of the compounds I, preferably with 0.5 mg/kg to 100 mg/kg of animal body weight per day.

Alternatively, the compounds I may be administered to animals parenterally, e.g., by intraruminal, intramuscular, intravenous or subcutaneous injection. The compounds I may be dispersed or dissolved in a physiologically acceptable carrier for subcutaneous injection. Alternatively, the compounds I may be formulated into an implant for subcutaneous administration. In addition, the compounds I may be transdermally administered to animals. For parenteral administration, the dosage form chosen should provide the animal with 0.01 mg/kg to 100 mg/kg of animal body weight per day of the compounds 1.

The compounds I may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the compounds 1.

In addition, the compounds I may be formulated as ear tags for animals, particularly quadrupeds e.g. cattle and sheep.

Oral solutions are administered directly.

Solutions for use on the skin are trickled on, spread on, rubbed in, sprinkled on or sprayed on.

Gels are applied to or spread on the skin or introduced into body cavities.

Pour-on formulations are poured or sprayed onto limited areas of the skin, the active compound penetrating the skin and acting systemically. Pour-on formulations are prepared by dissolving, suspending, or emulsifying the active compound in suitable skin-compatible solvents or solvent mixtures.

Emulsions can be administered orally, dermally or as injections.

Suspensions can be administered orally or topically/dermally.

Semi-solid preparations can be administered orally or topically/dermally.

For the production of solid preparations, the active compound is mixed with suitable excipients, if appropriate with addition of auxiliaries, and brought into the desired form.

The compositions which can be used in the invention can comprise generally from about 0.001 to 95% of the compound I.

Ready-to-use preparations contain the compounds acting against parasites, preferably ectoparasites, in concentrations of 10 ppm to 80% by weight, preferably from 0.1 to 65% by weight, more preferably from 1 to 50% by weight, most preferably from 5 to 40% by weight.

Preparations which are diluted before use contain the compounds acting against ectoparasites in concentrations of 0.5 to 90% by weight, preferably of 1 to 50% by weight.

Furthermore, the preparations comprise the compounds of formula I against endoparasites in concentrations of 10 ppm to 2% by weight, preferably of 0.05 to 0.9% by weight, very particularly preferably of 0.005 to 0.25% by weight.

Solid formulations which release compounds of the invention may be applied in total amounts of 10 mg/kg to 300 mg/kg, preferably 20 mg/kg to 200 mg/kg, most preferably 25 mg/kg to 160 mg/kg body weight of the treated animal in the course of three weeks.

### A. PREPARATION EXAMPLES

With appropriate modification of the starting materials, the procedures given in the synthesis description were used to obtain further compounds 1. The compounds obtained in this manner are listed in the table that follows, together with physical data.

The products shown below were characterized by melting point determination, by NMR spectroscopy or by the masses ([m/z]) or retention time (RT; [min.]) determined by HPLC-MS or HPLC spectrometry.

HPLC-MS=high performance liquid chromatography-coupled mass spectrometry;

HPLC method A: Shimadzu LC2010, Column: Waters XBridge C18, 150 mm×4.6 mm ID×5μ; Mobile Phase: A: water+0.1% TFA; B: acetonitrile+0.1% TFA; Temperature: 40° C.; Gradient: 10% B to 100% B in 5 min; 100% B 2 min; 10% B 3 min; Flow: 1.4 ml/min; Run Time: 10 min; PDA detector.

### Example 1: Synthesis of N-(3-(aminomethyl)-4-fluorophenyl)-5-(3,5-dichloro-4-fluorophenyl)-5-(trifluoromethyl)-4,5-dihydroisoxazol-3-amine

Step 1: To a solution of i-PrMgCl (98 mL, 196.4 mmol) in THF (450 mL) was added n-BuLi (79.5 mL, 178.6 mmol) dropwise at 0° C. and stirred additionally for 30 min at 0° C. A solution of 5-bromo-1,3-dichloro-2-fluorobenzene (40 g, 180 mmol) in THF (150 mL) was added dropwise to the mixture at 0° C. and stirred for 30 min at 0° C. under N₂. The mixture of 2,2,2-trifluoro-N-methoxy-N-methylacetamide (42 g, 270 mmol) in THF (100 mL) was added dropwise to the mixture at 0° C. Then it was stirred for 3 hrs at 0° C. under N₂. TLC (PE/EtOAc=5/1) indicated full conversion. The resulting mixture was poured in NH₄Cl aq. (500 mL) and extracted with MTBE (200 mL×2). The combined organic layers were washed with brine (200 mL), dried over sodium sulfate and concentrated to dryness. The residue was purified by column chromatography on silica gel (PE:EtOAc=1:0 to 5:1) to afford 1-(3,5-dichloro-4-fluorophenyl)-2,2,2-trifluoroethan-1-one (20 g, yield: 45.9%) as yellow oil.

Step 2: To a solution of 1-(3,5-dichloro-4-fluorophenyl)-2,2,2-trifluoroethan-1-one (49.4 g, 189.3 mmol) in THF (500 mL) was added diethyl(cyanomethyl)phosphonate (33.6 g, 189.8 mmol), Triethylamine (70 mL) and LiBr (16.5 g, 189.7 mmol) at 25° C. under N₂. Then the reaction mixture was heated to 70° C. and stirred for 3 hrs. TLC (PE/EtOAc=5/1) indicated full conversion. The resulting reaction mixture was filtrated, the filtrate poured into water (200 mL) and extracted with EtOAc (500 mL×2). The combined organic phase was washed with brine (100 mL), dried over sodium sulfate and concentrated to dryness. The crude product was purified by column chromatography on silica gel (PE:EtOAc=1:0 to 3:1) to afford (Z)-3-(3,5-dichloro-4-fluorophenyl)-4,4,4-trifluorobut-2-enenitrile (50 g, yield: 96.7%) as yellow oil.

¹H-NMR (400 MHz, CDCl₃): δ=7.50 (d, J=5.9 Hz, 2H), 6.28-6.26 (m, 1H).

Step 3: Note: Two batches were set-up simultaneously. To a solution of NaOMe (15.6 g, 302 mmol) in MeOH (306 mL) was added 1-hydroxyurea (10.4 g, 137.4 mmol) at 25° C. The solution of 3-(3,5-dichloro-4-fluorophenyl)-4,4,4-trifluorobut-2-enenitrile (25 g, 91.6 mmol) in MeOH (100 mL) was added dropwise to the reaction mixture at 0° C. and stirred for 3 hrs at 50° C. LCMS indicated full conversion. The resulting reaction mixture was poured into water (300 mL) and extracted with EtOAc (100 mL×2). The combined organic layers were washed with brine (100 mL), dried over Na₂SO₄ and concentrated to give 5-(3,5-dichloro-4-fluorophenyl)-5-(trifluoromethyl)-4,5-dihydroisoxazol-3-amine (33 g, yield: crude) as white solid and used in the next step without prior purification.

¹H-NMR (400 MHz, CD₃OD) δ=7.65 (d, J=6.3 Hz, 2H), 3.78-3.71 (m, 1H), 3.50-3.44 (m, 1H).

Step 4: To a solution of 5-bromo-2-fluorobenzaldehyde (50 g, 246 mmol) in toluene (500 mL) was added ethane-1,2-diol (45.8 g, 739 mmol) and TsOH (4.23 g, 24.6 mmol) at 25° C. The reaction mixture was stirred for 16 hrs at 130° C. under N₂. TLC (PE:EtOAc=10:1) indicated full conversion. The reaction was quenched with NaHCO₃ (800 mL) and extracted with EtOAc (400 mL×2). The combined organic layers were washed with brine (400 mL), dried over sodium sulfate and concentrated to dryness. The crude product was purified by column chromatography on silica gel (PE/EtOAc=1:0 to 10:1) to afford 2-(5-bromo-2-fluorophenyl)-1,3-dioxolane (35 g, yield: 57.6%) as white oil.

¹H-NMR (400 MHz, CDCl₃) δ=7.67 (dd, J=2.6, 6.2 Hz, 1H), 7.47-7.41 (m, 1H), 7.00-6.92 (m, 1H), 6.06-6.03 (m, 1H), 4.16-4.09 (m, 2H), 4.08-3.99 (m, 2H).

Step 5: The solution of 5-(3,5-dichloro-4-fluorophenyl)-5-(trifluoromethyl)-4,5-dihydroisoxazol-3-amine (5 g, 15.77 mmol) and 2-(5-bromo-2-fluorophenyl)-1,3-dioxolane (4.3 g, 17.35 mmol) in dioxane (100 mL) was added Pd(dba)₂ (907 mg, 1.577 mmol), XantPhos (1.83 g, 3.15 mmol), K₃PO₄ (6.695 g, 31.5 mmol) and stirred at 120° C. for 16 hrs under N₂. TLC (PE:EtOAc=3:1) indicated full conversion of the starting material. The resulting mixture was poured into water (200 mL), extracted with EtOAc (100 mL×2). The combined organic layers were washed with brine (100 mL), dried over Na₂SO₄ and concentrated to afford the crude product and was further purified by column chromatography (PE:EtOAc=1:0 to 0:1) to yield N-(3-(1,3-dioxolan-2-yl)-4-fluorophenyl)-5-(3,5-dichloro-4-fluorophenyl)-5-(trifluoromethyl)-4,5-dihydroisoxazol-3-amine (3.5 g, yield: 46%) as a yellow solid.

¹H-NMR (400 MHz, CD₃OD) δ=7.72-7.68 (m, 2H), 7.65 (d, J=6.3 Hz, 1H), 7.61-7.57 (m, 1H), 7.44-7.38 (m, 1H), 7.03 (dd, J=9.1, 9.8 Hz, 1H), 6.02-5.98 (m, 1H), 4.17-4.06 (m, 2H), 4.06-3.98 (m, 2H), 3.97-3.90 (m, 1H), 3.68 (d, J=16.8 Hz, 1H)

Step 6: To a solution of N-(3-(1,3-dioxolan-2-yl)-4-fluorophenyl)-5-(3,5-dichloro-4-fluorophe-nyl)-5-(trifluoromethyl)-4,5-dihydroisoxazol-3-amine (3.5 g, 7.25 mmol) in THF (35 mL) was added HCl aq. (35 mL, 4N) at 25° C. The reaction mixture was stirred for 2 hrs at 40° C. TLC (PE:EtOAc=3:1) indicated full conversion. The reaction mixture was quenched with NaHCO₃ aq. (240 mL), extracted with EtOAc (30 mL×3). The combined organic layers were washed with brine (30 mL), dried over Na₂SO₄ and concentrated to dryness. The crude product was purified by the column chromatography (PE/EtOAc=1:0 to 1:1) to afford 5-((5-(3,5-dichloro-4-fluorophenyl)-5-(trifluoromethyl)-4,5-dihydroisoxazol-3-yl)amino)-2-fluorobenzaldehyde (3 g, yield: 94.2%) as yellow oil.

¹H-NMR (400 MHz, DMSO-d₆) δ=10.22-10.19 (m, 1H), 9.62-9.54 (m, 1H), 7.96-7.90 (m, 1H), 7.87-7.81 (m, 2H), 7.69-7.62 (m, 1H), 7.41-7.33 (m, 1H), 4.00-3.93 (m, 1H), 3.90-3.82 (m, 1H)

Step 7: To a solution of 5-((5-(3,5-dichloro-4-fluorophenyl)-5-(trifluoromethyl)-4,5-dihydro-isoxazol-3-yl)amino)-2-fluorobenzaldehyde (3 g, 6.8 mmol) in EtOH (120 mL) was added the NH₄OAc (6.3 g, 82.2 mmol) and NH₃ (60 mL, 7N in MeOH) at 25° C. The reaction mixture was stirred for 1 hr at 25° C. Then NaBH₄ (1 g, 27.2 mmol) was added to the reaction mixture at 0° C. and stirred 2 hrs at 0-25° C. TLC (EtOAc:MeOH=1:1) indicated full conversion. The reaction mixture was quenched with NH₄Cl aq. (100 mL) and extracted with EtOAc (100 mL×3). The combined organic layers were concentrated to dryness and purified by column chromatography (PE/EtOAc=1:0 to 0:1) to afford N-(3-(aminomethyl)-4-fluorophenyl)-5-(3,5-dichloro-4-fluoro-phenyl)-5-(trifluoromethyl)-4,5-dihydroisoxazol-3-amine (1.6 g, yield: 53.3%) as a white solid.

¹H-NMR (400 MHz, CD₃OD) δ=7.75-7.66 (m, 2H), 7.50-7.41 (m, 1H), 7.30-7.21 (m, 1H), 7.06-6.96 (m, 1H), 3.98-3.92 (m, 1H), 3.86-3.81 (m, 2H), 3.73-3.65 (m, 1H), 2.05-2.02 (m, 1H)

### Example 2: Synthesis of N-(3-(aminomethyl)-4-fluorophenyl)-5-(3,5-dichloro-4-fluorophenyl)-N-ethyl-5-(trifluoromethyl)-4,5-dihydroisoxazol-3-amine

Step 1: To a solution of N-(3-(1,3-dioxolan-2-yl)-4-fluorophenyl)-5-(3,5-dichloro-4-fluorophe-nyl)-5-(trifluoromethyl)-4,5-dihydroisoxazol-3-amine (3.7 g, 7.7 mmol) in THF (120 mL) was added NaH (368 mg, 9.2 mmol) at 0° C. The reaction was stirred for 0.5 hrs at 0° C. under N₂. Then EtOTf (1.64 g, 9.2 mmol) was added dropwise to the mixture at 0° C. and stirred for 4 hrs at 0-25° C. TLC (PE:EtOAc=3:1) indicated full conversion. The resulting solution was quenched with NH₄Cl aq. (100 mL) and extracted with EtOAc (50 mL×2). The combined organic layers were washed with brine (100 mL), dried over sodium sulfate and concentrated to dryness. The crude product was purified by column chromatography on silica gel (PE:EtOAc=1:0 to 1:1) to afford N-(3-(1,3-dioxolan-2-yl)-4-fluorophenyl)-5-(3,5-dichloro-4-fluorophenyl)-N-ethyl-5-(tri-fluoromethyl)-4,5-dihydroisoxazol-3-amine (4 g, yield: crude) as yellow oil.

Step 2: To a solution of N-(3-(1,3-dioxolan-2-yl)-4-fluorophenyl)-5-(3,5-dichloro-4-fluorophe-nyl)-N-ethyl-5-(trifluoromethyl)-4,5-dihydroisoxazol-3-amine (4 g, 7.8 mmol) in THF (40 mL) was added HCl aq. (40 mL, 4N) at 25° C. The reaction mixture was stirred for 3 hrs at 40° C. TLC (PE:EtOAc=3:1) indicated full conversion of the starting material. The reaction was quenched with NaHCO₃ aq. (200 mL) and extracted with EtOAc (50 mL×2). The combined organic layers were washed with brine (100 mL), dried over sodium sulfate and concentrated to dryness. The crude product was purified by column chromatography on silica gel (PE:EtOAc=1:0 to 3:1) to afford 5-((5-(3,5-dichloro-4-fluorophenyl)-5-(trifluoromethyl)-4,5-dihydroisoxazol-3-yl)(ethyl)amino)-2-fluorobenzaldehyde (3 g, yield: 82.1%) as yellow oil.

¹H-NMR (400 MHz, CDCl₃) δ=10.38-10.36 (m, 1H), 7.64 (dd, J=2.9, 5.9 Hz, 1H), 7.48-7.45 (m, 2H), 7.43-7.37 (m, 1H), 7.27 (s, 1H), 3.64 (br s, 2H), 3.54-3.47 (m, 1H), 3.14-3.05 (m, 1H), 1.17 (t, J=7.1 Hz, 3H)

Step 3: To a solution of 5-((5-(3,5-dichloro-4-fluorophenyl)-5-(trifluoromethyl)-4,5-dihydrois-oxazol-3-yl)(ethyl)amino)-2-fluorobenzaldehyde (1.7 g, 3.65 mmol) in EtOH (60 mL) was added NH₄OAc (3.4 g, 43.8 mmol) and NH₃ (50 mL, 7N in MeOH) at 0° C. The reaction mixture was stirred for 1 hr at 25° C. Then NaBH₃CN (920 mg, 14.6 mmol) was added at 0° C. and stirred for 16 hrs at 50° C. TLC (PE:EtOAc=1:1) indicated full conversion of the starting material. The resulting solution was quenched with NH₄Cl aq. (100 mL) and extracted with EtOAc (50 mL×2). The combined organic layers were washed with brine (100 mL), dried over sodium sulfate and concentrated to dryness. The crude product was purified by column chromatography on silica gel (PE:EtOAc=1:0 to 0:1) to afford N-(3-(aminomethyl)-4-fluorophenyl)-5-(3,5-dichloro-4-fluorophenyl)-N-ethyl-5-(trifluoromethyl)-4,5-dihydroisoxazol-3-amine (1.5 g, yield: 87.6%) as yellow oil.

¹H-NMR (400 MHz, CD₃OD) δ=7.66-7.61 (m, 2H), 7.33-7.26 (m, 1H), 7.11 (s, 2H), 3.85 (s, 2H), 3.69-3.64 (m, 2H), 3.64-3.61 (m, 1H), 3.46-3.40 (m, 1H), 1.17-1.12 (m, 3H)

### Example 3: Synthesis of N-(3-(aminomethyl)-4-fluorophenyl)-5-(3,5-dichloro-4-fluorophenyl)-N-(ethoxymethyl)-5-(trifluoromethyl)-4,5-dihydroisoxazol-3-amine

Step 1: To a solution of 5-((5-(3,5-dichloro-4-fluorophenyl)-5-(trifluoromethyl)-4,5-dihydrois-oxazol-3-yl)amino)-2-fluorobenzaldehyde (3.8 g, 8.15 mmol) in THF (120 mL) was added NaH (392 mg, 9.79 mmol) at 0° C. The reaction mixture was stirred for 1 hr at 0° C. under N₂. Then (chloromethoxy)ethane (925 mg, 9.79 mmol) was added dropwise to the mixture at 0° C. and stirred for 4 hrs at 0-25° C. TLC (PE:EtOAc=3:1) indicated full conversion. The resulting solution was quenched with NH₄Cl aq. (100 mL) and extracted with EtOAc (50 mL×2). The combined organic layers were washed with brine (100 mL), dried over sodium sulfate and concentrated to dryness. The residue was purified by column chromatography on silica gel (PE:EtOAc=1:0 to 1:1) to afford 5-((5-(3,5-dichloro-4-fluorophenyl)-5-(trifluoromethyl)-4,5-dihydrois-oxazol-3-yl)(ethoxymethyl)amino)-2-fluorobenzaldehyde (2.79 g, yield: 68.22%) as yellow oil.

¹H-NMR (400 MHz, CD₃OD) δ=7.66 (d, J=6.3 Hz, 2H), 7.52 (dd, J=2.6, 6.1 Hz, 1H), 7.35-7.26 (m, 1H), 7.13 (t, J=9.3 Hz, 1H), 5.75 (s, 1H), 4.92 (s, 2H), 4.02-3.87 (m, 1H), 3.87-3.71 (m, 1H), 3.61-3.56 (m, 2H), 1.18 (t, J=6.6 Hz, 3H).

Step 2: To a solution of 5-((5-(3,5-dichloro-4-fluorophenyl)-5-(trifluoromethyl)-4,5-dihydro-isoxazol-3-yl)(ethoxymethyl)amino)-2-fluorobenzaldehyde (2.76 g, 5.56 mmol) in EtOH (90 mL) was added NH₄OAc (6 g, 77.9 mmol) and NH₃ (60 mL, 7N in MeOH) at 0° C. and stirred for 2 hrs. Then NaBH₃CN (1.4 g, 22.24 mmol) was added to the reaction at 0° C. and stirred for 16 hrs at 50° C. TLC (PE:EtOAc=0:1) indicated full conversion. The resulting solution was quenched with NH₄Cl aq. (100 mL) and extracted with EtOAc (50 mL×2). The combined organic layers were washed with brine (100 mL), dried over sodium sulfate and concentrated to dryness. The residue was purified by column chromatography on silica gel (PE:EtOAc=1:0 to 0:1) to afford N-(3-(aminomethyl)-4-fluorophenyl)-5-(3,5-dichloro-4-fluorophenyl)-N-(ethoxymethyl)-5-(trifluoromethyl)-4,5-dihydroisoxazol-3-amine (2.5 g, yield: 90.29%) as yellow oil.

¹H-NMR (400 MHz, CD₃OD) δ=7.67 (d, J=6.1 Hz, 2H), 7.38 (dd, J=2.7, 6.6 Hz, 1H), 7.29-7.24 (m, 1H), 7.18-7.10 (m, 1H), 4.94-4.91 (m, 2H), 3.93-3.88 (m, 1H), 3.83 (br s, 1H), 3.69-3.64 (m, 1H), 3.61-3.55 (m, 2H), 1.18 (t, J=7.1 Hz, 3H).

### Example 4: Synthesis of ethyl (4-(aminomethyl)-3-fluorophenyl)(5-(3,5-dichloro-4-fluorophenyl)-5-(trifluoromethyl)-4,5-dihydroisoxazol-3-yl)carbamate

Step 1: To a solution of 5-(3,5-dichloro-4-fluorophenyl)-5-(trifluoromethyl)-4,5-dihydroisoxazol-3-amine (10 g, 31.5 mmol) in dioxane (150 mL) was added 2-bromo-1-fluoro-4-iodobenzene (11.3 g, 37.8 mmol), Pd₂(dba)₃ (1.4 g, 1.57 mmol), XantPhos (1.8 g, 3.15 mmol), Cs₂CO₃ (20 g, 63 mmol) at 20° C. under N₂ and stirred at 80° C. under N₂ for 16 hrs. TLC (PE:EtOAc=3:1) indicated full conversion. The resulting reaction mixture was quenched with H₂O (200 mL), extracted with EtOAc (80 mL×3). The combined organic layers were washed with brine (100 mL), dried over Na₂SO₄ and concentrated to dryness. The crude product was purified by column chromatography on silica gel (PE:EtOAc=100:0˜17:83) to afford N-(3-bromo-4-fluorophenyl)-5-(3,5-dichloro-4-fluorophenyl)-5-(trifluoromethyl)-4,5-dihydroisoxazol-3-amine (8.2 g, yield: 52.9%) as a yellow solid.

¹H-NMR (400 MHz, CDCl₃) δ=(dd, J=5.75, 2.75 Hz, 1H) 7.56 (d, J=6.00 Hz, 2H) 7.20-7.26 (m, 1H) 7.01-7.16 (m, 1H) 5.85 (s, 1H) 3.90 (d, J=16.01 Hz, 1H) 3.50 (d, J=16.01 Hz, 1H).

Step 2: To a solution of N-(3-bromo-4-fluorophenyl)-5-(3,5-dichloro-4-fluorophenyl)-5-(trifluoro-methyl)-4,5-dihydroisoxazol-3-amine (4 g, 8.16 mmol) in THF (40 mL) was added NaH (490 mg, 12.2 mmol) at 0° C. The reaction mixture was stirred for 0.5 hrs at 0° C. under N₂. Then ethyl carbonochloridate (2.67 g, 24.5 mmol) in THF (4 mL) was added dropwise to the reaction at 0° C. and additionally stirred for 2 hrs at 0° C. TLC (PE:EtOAc=3:1) indicated full conversion. The resulting solution was quenched with aq. NH₄Cl (100 mL) and extracted with EtOAc (50 mL×2). The combined organic layers were washed with brine (100 mL), dried over sodium sulfate and concentrated to dryness. The residue was purified by column chromatography on silica gel (PE:EtOAc=1:0 to 1:1) to afford ethyl (3-bromo-4-fluorophenyl)(5-(3,5-dichloro-4-fluorophenyl)-5-(trifluoromethyl)-4,5-dihydroisoxazol-3-yl)carbamate (4 g, yield: 86.9%) as yellow oil.

¹H-NMR (400 MHz, CDCl₃) δ=7.51 (d, J=6.00 Hz, 2H), 7.43 (dd, J=5.94, 2.31 Hz, 1H), 7.13-7.23 (m, 2H), 4.40 (d, J=18.26 Hz, 1H), 4.21 (q, J=7.05 Hz, 2H), 4.03 (d, J=18.39 Hz, 1H), 1.25-1.29 (m, 3H).

Step 3: To a solution of ethyl (3-bromo-4-fluorophenyl)(5-(3,5-dichloro-4-fluorophenyl)-5-(tri-fluoromethyl)-4,5-dihydroisoxazol-3-yl)carbamate (4 g, 7.1 mmol) in toluene (40 mL) was added (((tert-butoxycarbonyl)amino)methyl)trifluoroborate (2.1 g, 10.65 mmol), tBu₃P—Pd-G2 (386 mg, 0.71 mmol), K₂CO₃ (2M, 7.1 ml, 14.2 mmol) at 25° C. The reaction was stirred at 80° C. for 12 h. HPLC indicated 51% conversion toward the desired product. The reaction mixture was quenched with H₂O (40 ml), the filtrate was extracted with EtOAc (40 mL×3), the combined organic layers were washed with brine (40 mL), dried over Na₂SO₄, concentrated to dryness and the crude product was further purified by column chromatography on silica gel (PE:EtOAc=100:0˜95:5) to afford ethyl (3-(((tert-butoxycarbonyl)amino)methyl)-4-fluorophenyl)(5-(3,5-di-chloro-4-fluorophenyl)-5-(trifluoromethyl)-4,5-dihydroisoxazol-3-yl)carbamate (4 g, yield 91.9%) as a white solid.

¹H-NMR (400 MHz, CDCl₃) δ=7.51 (d, J=6.00 Hz, 2H), 7.21 (br d, J=5.38 Hz, 1H), 7.05-7.14 (m, 2H), 4.31-4.47 (m, 3H), 4.18 (q, J=7.00 Hz, 2H), 4.04 (d, J=18.39 Hz, 1H), 1.44 (s, 9H), 1.19 (t, J=7.13 Hz, 3H).

Step 4: A solution of ethyl (3-(((tert-butoxycarbonyl)amino)methyl)-4-fluorophenyl)(5-(3,5-di-chloro-4-fluorophenyl)-5-(trifluoromethyl)-4,5-dihydroisoxazol-3-yl)carbamate (4 g, 6.5 mmol) in HCl/EtOAc (50 mL) was stirred at 25° C. for 2 hrs. TLC (PE:EtOAc=1:1) indicated full conversion. The resulting reaction mixture was poured into NaHCO₃ (100 mL) and extracted with EtOAc (50 mL×2). The combined organic layers were washed with brine (100 mL), dried over Na₂SO₄ and concentrated to dryness to afford the crude product, which was further purified by column chromatography (PE:EtOAc=1:0 to 0:1) to afford ethyl (3-(aminomethyl)-4-fluoro-phenyl)(5-(3,5-dichloro-4-fluorophenyl)-5-(trifluoromethyl)-4,5-dihydroisoxazol-3-yl)carbamate (2.015 g, yield: 60.3%) as a white solid.

¹H-NMR (400 MHz, CDCl₃) δ=7.51 (d, J=6.02 Hz, 2H), 7.25 (dd, J=6.53, 1.76 Hz, 1H), 7.05-7.12 (m, 2H), 4.41 (d, J=18.32 Hz, 1H), 4.19 (q, J=7.07 Hz, 2H), 4.04 (d, J=18.32 Hz, 1H), 3.94 (s, 2H), 1.20 (t, J=7.09 Hz, 3H).

### Example 5: Synthesis of N-(3-(1,3-dioxolan-2-yl)-4-fluorophenyl)-5-(3,5-dichloro-4-fluorophenyl)-5-(trifluoromethyl)-4,5-dihydroisoxazol-3-amine

Step 1: To a solution of 5-(3,5-dichloro-4-fluorophenyl)-5-(trifluoromethyl)-4,5-dihydroisoxazol-3-amine (10 g, 31.53 mmol) in dioxane (150 mL) was added 2-(5-bromo-2-fluorophenyl)-1,3-dioxolane (12 g, 37.9 mmol), Pd₂(dba)₃ (2 g, 3.15 mmol), Xantphos (2.74 g, 4.7 mmol), K₃PO₄ (13.4 g, 63 mmol) at 20° C. under N₂ and was stirred at 80° C. under N₂ for 16 hrs. TLC (PE:EtOAc=3:1) indicated full conversion. The resulting reaction mixture was quenched with H₂O (200 mL), extracted with EtOAc (80 mL×3). The combined organic layers were washed with brine (100 mL), dried over Na₂SO₄ and concentrated to dryness. The crude product was further purified by MPLC to afford N-(3-(1,3-dioxolan-2-yl)-4-fluorophenyl)-5-(3,5-dichloro-4-fluoro-phenyl)-5-(trifluoromethyl)-4,5-dihydroisoxazol-3-amine (1.82 g, yield: 11.96%) as a yellow solid.

¹H-NMR (400 MHz, CDCl₃) δ=7.55 (d, J=6.00 Hz, 2H), 7.39-7.47 (m, 2H), 7.02 (t, J=9.19 Hz, 1H), 6.05 (s, 1H), 6.02 (s, 1H), 4.11-4.20 (m, 2H), 4.00-4.10 (m, 2H), 3.86 (d, J=16.01 Hz, 1H), 3.45 (d, J=16.13 Hz, 1H).

### Example 6: Synthesis of 5-(3,5-dichloro-4-fluorophenyl)-N-(4-fluoro-3-((methylami-no)methyl)phenyl)-5-(trifluoromethyl)-4,5-dihydroisoxazol-3-amine

Step 1: To a solution of 5-((5-(3,5-dichloro-4-fluorophenyl)-5-(trifluoromethyl)-4,5-dihydro-isoxazol-3-yl)amino)-2-fluorobenzaldehyde (3.1 g, 7.08 mmol) in MeOH (120 mL) was added MeNH₂ (7.3 g, ˜30% in EtOH, 70.8 mmol) at 25° C. and stirred for 16 hrs at 65° C. Then NaBH₄ (1.1 g, 28.3 mmol) was added to the reaction mixture at 0° C. The reaction was stirred for 4 hrs. TLC (EtOAc:MeOH=1:1) indicated full conversion. The resulting reaction mixture was quenched with NH₄Cl aq. (100 mL) and extracted with DCM (100 mL×3). The combined organic layers were concentrated and purified by column chromatography on silica gel (PE:EtOAc=1:0 to 0:1) to afford 5-(3,5-dichloro-4-fluorophenyl)-N-(4-fluoro-3-((methylamino)methyl)phenyl)-5-(trifluoromethyl)-4,5-dihydroisoxazol-3-amine (2.4 g, yield: 74.9%) as a white solid.

¹H-NMR (400 MHz, CD₃OD) δ=7.78-7.73 (m, 1H), 7.71 (d, J=6.3 Hz, 2H), 7.44 (s, 1H), 7.19 (t, J=9.2 Hz, 1H), 4.27-4.22 (m, 2H), 4.02-3.95 (m, 1H), 3.78-3.70 (m, 1H), 2.76-2.73 (m, 3H).

### Example 7: Synthesis of N-(3-(aminomethyl)-4-methylphenyl)-5-(3,5-dichloro-4-fluorophenyl)-5-(trifluoromethyl)-4,5-dihydroisoxazol-3-amine

Step 1: To a solution of 2-bromo-5-iodobenzonitrile (10 g, 32.48 mmol) in THF (100 mL) was added BH₃·THF (77.94 mL, 77.94 mmol) dropwise at 0° C. under N₂ and upon full addition additionally stirred at 70° C. for 16 hrs. TLC (PE:EA=5:1) indicated full conversion. The mixture was poured into HCl aq. (1M, 50 mL) at 0° C. and stirred at 20° C. for 20 min. The pH of the mixture was adjusted to 10 with NaOH to afford the (2-bromo-5-iodophenyl)methanamine (10 g, yield: crude) in the solution. It was used directly without further purification.

Step 2: To a solution of (2-bromo-5-iodophenyl)methanamine (10 g, 32.06 mmol) in MeCN (100 mL) was added Boc₂O (20.99 g, 96.17 mmol) at 0° C. under N₂ and upon warming to 20° C. stirred for additional 16 hrs. TLC (PE:EA=5:1) indicated full conversion. The reaction mixture was poured into H₂O (200 mL) and extracted with EtOAc (90 ml×3). The combined organic phase was washed with brine (180 ml×3), dried over Na₂SO₄, filtered and concentrated to afford the crude product, which was further purified by column chromatography on silica gel (PE:EtOAc=1:0˜5:1) to afford tert-butyl (2-bromo-5-iodobenzyl)carbamate (10 g, yield: 75.7%) as a white solid.

¹H-NMR (400 MHz, CD₃OD) δ=1.47 (s, 9H), 4.23 (s, 2H), 7.31 (d, J=8.38 Hz, 1H), 7.49 (dd, J=8.25, 1.50 Hz, 1H), 7.63 (s, 1H).

Step 3: To a mixture of tert-butyl (2-bromo-5-iodobenzyl)carbamate (10 g, 24.27 mmol) in dioxane (100 mL) was added 5-(3,5-dichloro-4-fluorophenyl)-5-(trifluoromethyl)-4,5-dihydro-isoxazol-3-amine (5.92 g, 18.67 mmol), K₃PO₄ (11.89 g, 56 mmol), Pd₂(dba)₃ (1.71 g, 1.87 mmol) and XantPhos (2.16 g, 3.74 mmol) at 25° C. under N₂ and was stirred at 120° C. for 16 hrs. LCMS indicated full conversion. The mixture was poured into H₂O (200 mL) and extracted with EtOAc (100 ml×3). The combined organic phase was washed with brine (150 ml×3), dried over Na₂SO₄, filtered and concentrated to afford the crude product. The crude product was purified by column chromatography on silica gel (PE:EtOAc=1:0˜3:1) to yield tert-butyl (2-bromo-5-((5-(3,5-di-chloro-4-fluorophenyl)-5-(trifluoromethyl)-4,5-dihydroisoxazol-3-yl)amino)benzyl)carbamate (5.27 g, yield: 36.1%) as brown oil.

¹H-NMR (400 MHz, CDCl₃) δ=1.46 (s, 9H), 3.41 (br d, J=16.38 Hz, 1H), 3.82 (br d, J=16.38 Hz, 1H), 4.25 (d, J=6.24 Hz, 2H), 5.25 (br s, 1H), 7.00 (br s, 1H), 7.14-7.23 (m, 2H), 7.32 (br d, J=7.95 Hz, 1H), 7.53 (d, J=5.99 Hz, 2H).

Step 4: To a mixture of tert-butyl (2-bromo-5-((5-(3,5-dichloro-4-fluorophenyl)-5-(trifluoro-methyl)-4,5-dihydroisoxazol-3-yl)amino)benzyl)carbamate (5.26 g, 8.75 mmol) in dioxane (55 mL) and H₂O (11 mL) was added methylboronic acid (1.57 g, 26.25 mmol), Na₂CO₃ (2.78 g, 26.25 mmol), PdCl₂(dppf) (0.64 g, 0.88 mmol) at 25° C. under N₂ and was stirred at 110° C. for 16 hrs. LCMS indicated full conversion. The mixture was poured into H₂O (200 mL), extracted with EtOAc (100 ml×3). The combined organic phase was washed with brine (150 ml×3), dried over Na₂SO₄, filtered and concentrated to afford the crude product. The product was further purified by column chromatography on silica gel (PE:EtOAc=1:0˜3:1) to yield tert-butyl (5-((5-(3,5-dichloro-4-fluorophenyl)-5-(trifluoromethyl)-4,5-dihydroisoxazol-3-yl)amino)-2-methylbenzyl)carbamate (5.1 g, yield: crude) as a brown solid.

¹H-NMR (400 MHz, CDCl₃) δ=1.47 (s, 10H), 2.23 (s, 3H), 3.44 (br d, J=16.13 Hz, 1H), 3.85 (br d, J=16.13 Hz, 1H), 4.17-4.30 (m, 2H), 4.83 (br s, 1H), 6.45 (br s, 1H), 7.04 (d, J=8.13 Hz, 1H), 7.12-7.21 (m, 2H), 7.54 (d, J=6.00 Hz, 2H).

Step 5: To a solution of tert-butyl (5-((5-(3,5-dichloro-4-fluorophenyl)-5-(trifluoromethyl)-4,5-dihydroisoxazol-3-yl)amino)-2-methylbenzyl)carbamate (2.0 g, 3.73 mmol) in EtOAc/HCl (25 mL) was added at 25° C. under N₂ and was stirred at 25° C. for 16 hrs. TLC (PE:EA=5:1) indicated full conversion. The mixture was filtered, washed with MTBE (25 mL) and concentrated to afford N-(3-(aminomethyl)-4-methylphenyl)-5-(3,5-dichloro-4-fluorophenyl)-5-(trifluoromethyl)-4,5-dihydroisoxazol-3-amine hydrochloride (2.0 g, purity: 99%, 1.9 g delivered as hydrochloride) as a white solid.

¹H-NMR (400 MHz, CD₃OD) δ=2.35 (s, 3H), 3.74 (d, J=16.81 Hz, 1H), 3.99 (d, J=16.81 Hz, 1H), 4.12 (s, 2H), 7.17-7.26 (m, 2H), 7.56 (d, J=2.13 Hz, 1H), 7.71 (d, J=6.15 Hz, 2H).

### Example 8: Synthesis of N-(3-(aminomethyl)-4-chlorophenyl)-5-(3,5-dichloro-4-fluorophenyl)-5-(trifluoromethyl)-4,5-dihydroisoxazol-3-amine

Step 1: To a solution of 5-bromo-2-chlorobenzonitrile (15.0 g, 69.30 mmol) in THF (150 mL) was added dropwise BH₃THF (166.32 mL, 166.32 mmol) at 0° C. under N₂. Then the mixture was stirred at 70° C. for 16 hrs. LCMS indicated full conversion of the starting material. The mixture was poured into HCl (1M, 50 mL) and stirred at 20° C. for 20 min. The pH of the mixture was adjusted to 10 with NaOH to afford (5-bromo-2-chlorophenyl)methanamine (15 g) as a white liquid which was used directly without further purification.

Step 2: To a mixture of (5-bromo-2-chlorophenyl)methanamine (15 g, 68.03 mmol) in MeCN (150 mL) was added Boc₂O (44.55 g, 204.9 mmol) at 0° C. under N₂ and additionally stirred 16 hrs at 20° C. LCMS indicated full conversion of the starting material. The mixture was poured into H₂O (300 mL) and extracted with EtOAc (100 mL×3). The combined organic phase was washed with brine (200 mL), dried over Na₂SO₄, filtered and concentrated to afford the crude product, which was further purified by column chromatography on silica gel (PE:EtOAc=1:0˜5:1) to yield tert-butyl (5-bromo-2-chlorobenzyl)carbamate (10 g, yield: 45.8%) as a white solid.

¹H-NMR (400 MHz, CDCl₃) 5=1.47 (s, 9H), 4.37 (br d, J=5.75 Hz, 2H), 5.01 (br s, 1H), 5.30 (s, 1H), 7.15-7.25 (m, 1H), 7.34 (dd, J=8.50, 2.25 Hz, 1H), 7.51 (d, J=2.25 Hz, 1H).

Step 3: To a mixture of tert-butyl (5-bromo-2-chlorobenzyl)carbamate (5.6 g, 17.47 mmol) in dioxane (57 mL) was added 5-(3,5-dichloro-4-fluorophenyl)-5-(trifluoromethyl)-4,5-dihydro-isoxazol-3-amine (3.73 g, 11.65 mmol), K₃PO₄ (7.47 g, 34.94 mmol), Pd₂(dba)₃ (1.07 g, 1.17 mmol) and Xantphos (1.36 g, 2.33 mmol) at 25° C. under N₂ and was stirred at 110° C. for 16 hrs. LCMS indicated full conversion. The mixture was poured into H₂O (200 mL), extracted with EtOAc (100 mL×3). The combined organic phase was washed with brine (150 mL×3), dried over Na₂SO₄, filtered and concentrated to afford the crude product. The product was further purified by column chromatography on silica gel (PE:EtOAc=1:0˜2:1) to yield tert-butyl (2-chloro-5-((5-(3,5-dichloro-4-fluorophenyl)-5-(trifluoromethyl)-4,5-dihydroisoxazol-3-yl)amino)benzyl)carbamate (4.3 g, yield: 44.4%) as brown oil.

¹H-NMR (400 MHz, CDCl₃) δ=1.38 (s, 8H), 3.37 (br d, J=16.26 Hz, 1H), 3.78 (br d, J=16.13 Hz, 1H), 4.24 (d, J=6.38 Hz, 2H), 5.06 (br s, 1H), 7.15 (br d, J=8.50 Hz, 2H), 7.22-7.28 (m, 1H), 7.47 (d, J=6.00 Hz, 2H).

Step 4: To a solution of tert-butyl (2-chloro-5-((5-(3,5-dichloro-4-fluorophenyl)-5-(trifluoro-methyl)-4,5-dihydroisoxazol-3-yl)amino)benzyl)carbamate (3.6 g, 6.47 mmol) in EtOAc/HCl (36 mL) was added at 25° C. under N₂. Then the mixture was stirred at 25° C. for 3 hrs. TLC (PE EA=2:1) indicated full conversion. The mixture was concentrated to afford N-(3-(aminomethyl)-4-chlorophenyl)-5-(3,5-dichloro-4-fluorophenyl)-5-(trifluoromethyl)-4,5-dihydro-isoxazol-3-amine (2.5 g, yield: 47%) as a white solid.

¹H-NMR (400 MHz, DMSO-ds) 5=3.85-3.91 (m, 1H), 4.01 (s, 1H), 4.05 (br s, 2H), 7.45 (s, 2H), 7.65 (s, 1H), 7.82 (d, J=6.27 Hz, 2H), 8.47 (br s, 3H), 9.91 (s, 1H).

### Example 9: Synthesis of N-(3-(aminomethyl)-4-(trifluoromethoxy)phenyl)-5-(3,5-dichloro-4-fluorophenyl)-5-(trifluoromethyl)-4,5-dihydroisoxazol-3-amine

Step 1: To a solution of 5-bromo-2-(trifluoromethoxy)benzonitrile (10 g, 37.59 mmol) in THF (100 mL) was added BH₃THF (90.22 mL, 90.22 mmol) dropwise at 0° C. under N₂ and was additionally stirred at 70° C. for 16 h. LCMS indicated full conversion. The mixture was poured into HCl (1M, 50 mL) and stirred at 20° C. for 20 min. The pH of the mixture was adjusted to 10 with NaOH. Then to afford (5-bromo-2-(trifluoromethoxy)phenyl)methanamine (10 g) as a white liquid which was directly used without further purification.

Step 2: To a solution of (5-bromo-2-(trifluoromethoxy)phenyl)methanamine (8 g, 29.62 mmol) in MeCN (80 mL) was added Boc₂O (19.40 g, 88.87 mmol) at 0° C. under N₂ and was stirred at 20° C. for 16 hrs. LCMS indicated full conversion o. The mixture was poured into H₂O (300 mL) and extracted with EtOAc (100 mL×3). The combined organic phase was washed with brine (200 mL), dried over Na₂SO₄, filtered and concentrated to afford the crude product, which was further purified by column chromatography on silica gel (PE:EtOAc=1:0˜5:1) to yield tert-butyl (5-bro-mo-2-(trifluoromethoxy)benzyl)carbamate (12 g, yield: crude) as brown oil.

¹H-NMR (400 MHz, CDCl₃) δ=1.39-1.45 (m, 9H), 2.00 (s, 1H), 4.33 (br d, J=6.15 Hz, 2H), 7.07 (dd, J=8.72, 1.32 Hz, 1H), 7.38 (dd, J=8.72, 2.32 Hz, 1H), 7.52 (d, J=2.38 Hz, 1H).

Step 3: To a mixture of tert-butyl (5-bromo-2-(trifluoromethoxy)benzyl)carbamate (10.2 g, 27.64 mmol) in dioxane (102 mL) was added 5-(3,5-dichloro-4-fluorophenyl)-5-(trifluoromethyl)-4,5-dihydroisoxazol-3-amine (5.84 g, 18.43 mmol), K₃PO₄ (11.72 g, 55.28 mmol), Pd₂(dba)₃ (1.69 g, 1.84 mmol) and Xantphos (2.13 g, 3.69 mmol) at 25° C. under N₂ and was stirred at 110° C. for 16 hrs. LCMS indicated full conversion. The mixture was poured into H₂O (200 mL), extracted with EtOAc (100 mL×3). The combined organic phase was washed with brine (150 mL), dried over Na₂SO₄, filtered and concentrated to dryness to afford the crude product, which was further purified by column chromatography on silica gel (PE:EtOAc=1:0˜1:1) to yield tert-butyl (5-((5-(3,5-dichloro-4-fluorophenyl)-5-(trifluoromethyl)-4,5-dihydroisoxazol-3-yl)amino)-2-(trifluoro-methoxy)benzyl)carbamate (8.5 g, yield: crude) as brown oil.

¹H-NMR (400 MHz, CDCl₃) 5=1.47 (s, 9H), 3.48 (d, J=16.13 Hz, 1H), 3.83-3.95 (m, 1H), 4.33 (d, J=6.38 Hz, 2H), 7.16 (br d, J=8.88 Hz, 1H), 7.29-7.41 (m, 2H), 7.56 (d, J=6.00 Hz, 2H).

Step 4: A solution of tert-butyl (5-((5-(3,5-dichloro-4-fluorophenyl)-5-(trifluoromethyl)-4,5-dihydroisoxazol-3-yl)amino)-2-(trifluoromethoxy)benzyl)carbamate (5.1 g, 8.41 mmol) in EtOAc/HCl (51 mL) was stirred at 25° C. for 3 hrs. LCMs indicated full conversion. The mixture was poured into H₂O (200 mL) and extracted with EtOAc (50 mL×3). The combined organic phase was washed with brine (60 mL), dried over Na₂SO₄, filtered and concentrated to dryness to afford the crude product. The crude product was further purified by column chromatography on silica gel (PE:EtOAc=1:0˜1:1) to yield N-(3-(aminomethyl)-4-(trifluoromethoxy)phenyl)-5-(3,5-dichloro-4-fluorophenyl)-5-(trifluoromethyl)-4,5-dihydroisoxazol-3-amine (2.8 g, yield: 65%) as a white solid.

¹H-NMR (400 MHz, CD₃OD) δ=3.67-3.74 (m, 1H), 3.84 (s, 2H), 3.93-4.00 (m, 1H), 7.21 (dd, J=8.94, 1.44 Hz, 1H), 7.35 (dd, J=8.88, 2.75 Hz, 1H), 7.56 (d, J=2.75 Hz, 1H), 7.71 (d, J=6.25 Hz, 2H).

### Example 10: Synthesis of N-(4-(aminomethyl)-3-chlorophenyl)-5-(3-chloro-4-fluorophenyl)-5-(trifluoromethyl)-4,5-dihydroisoxazol-3-amine

To a solution of THF (150 mL) was added i-PrMgCl (39.5 mL, 78.95 mmol) and n-BuLi (28.7 mL, 71.77 mmol) at 0° C. The reaction mixture was stirred for 0.5 hrs at 0° C. Then 4-bromo-2-chloro-1-fluorobenzene (15 g, 71.77 mmol) was added and additionally stirred for 0.5 hrs at 0° C. Then the solution of 2,2,2-trifluoro-N-methoxy-N-methylacetamide (16.9 g, 107.66 mmol) in THF (150 mL) was added dropwise at 0° C. and additionally stirred for 2 hrs at 0-25° C. TLC (PE:EtOAc=5:1) indicated full conversion. The resulting mixture was quenched with NH₄Cl aq. (300 mL) and extracted with EtOAc (100 mL×3). The combined organic layers were washed with brine (100 mL), dried over sodium sulfate, concentrated to dryness and purified by column chromatography on silica gel (PE:EtOAc=1:0 to 1:1) to afford 1-(3-chloro-4-fluorophenyl)-2,2,2-trifluoroethan-1-one (9.4 g, yield: 58.3%) as yellow oil.

¹H-NMR (400 MHz, CD₃OD) δ=7.68 (dd, J=2.1, 7.2 Hz, 1H), 7.59-7.52 (m, 1H), 7.28 (t, J=8.9 Hz, 1H).

Step 2: To the mixture of 1-(3-chloro-4-fluorophenyl)-2,2,2-trifluoroethan-1-one (9.4 g, 41.4 mmol) in THF (50 mL) was added diethyl (cyanomethyl)phosphonate (7.3 g, 41.4 mmol) and TEA (11.5 mL, 82.8 mmol) at 25° C. A mixture of LiBr (3.6 g, 41.4 mmol) in THF (50 mL) was added to the reaction at 0° C. and stirred for 4 hrs at 70° C. TLC (PE:EtOAc=5:1) indicated full conversion. The resulting reaction mixture was poured into H₂O (100 mL) and extracted with EtOAc (100 mL×3). The combined organic layers were washed with brine (100 mL), dried over sodium sulfate, concentrated to dryness and purified by column chromatography on silica gel (PE:EtOAc=1:0 to 3:1) to afford 3-(3-chloro-4-fluorophenyl)-4,4,4-trifluorobut-2-enenitrile (9.1 g, yield: 88.07%) as yellow oil.

¹H-NMR (400 MHz, CDCl₃) δ=7.60 (dd, J=2.1, 6.6 Hz, 1H), 7.45 (ddd, J=2.3, 4.3, 8.5 Hz, 1H), 7.33-7.28 (m, 1H), 6.24-6.20 (m, 1H).

Step 3: To a solution of MeONa (6.5 g, 120.12 mmol) in MeOH (100 mL) was added 1-hydroxyurea (4.15 g, 54.6 mmol). Then a solution of 3-(3-chloro-4-fluorophenyl)-4,4,4-trifluorobut-2-enenitrile (9.1 g, 36.4 mmol) in MeOH (100 mL) was added dropwise at 0° C. The solution was stirred for 3 hrs at 50° C. TLC (PE:EtOAc=3:1) indicated full conversion. The resulting reaction mixture was poured into H₂O (200 mL) and extracted with EtOAc (100 mL×3). The combined organic layers were concentrated and purified by column chromatography on silica gel (PE:EtOAc=1:0 to 3:1) to afford 5-(3-chloro-4-fluorophenyl)-5-(trifluoromethyl)-4,5-dihydroisoxazol-3-amine (6 g, yield: 58.2%) as a white solid.

¹H-NMR (400 MHz, CD₃OD) δ=7.67 (dd, J=2.2, 6.9 Hz, 1H), 7.56-7.48 (m, 1H), 7.32 (t, J=8.8 Hz, 1H), 3.78-3.70 (m, 1H), 3.45 (d, J=16.9 Hz, 1H).

Step 4: To a solution of tert-butyl (5-bromo-2-chlorobenzyl)carbamate (8.5 g, 26.5 mmol) and 5-(3-chloro-4-fluorophenyl)-5-(trifluoromethyl)-4,5-dihydroisoxazol-3-amine (5 g, 17.67 mmol) in dioxane (100 mL) was added Pd₂(dba)₃ (1.62 g, 1.77 mmol), Xantphos (2.05 g, 3.53 mmol) and K₃PO₄ (11.24 g, 53.01 mmol) and was stirred at 110° C. for 16 hrs under N₂. TLC (PE:EtOAc=3:1) indicated full conversion. The reaction mixture was poured into H₂O (200 mL), extracted with EtOAc (100 mL×2). The combined organic layers were washed with brine (100 mL), dried over Na₂SO₄ and concentrated to dryness to afford the crude product, which was further purified by column chromatography on silica gel (PE:EtOAc=1:0 to 0:1) to afford tert-butyl (2-chloro-5-((5-(3-chloro-4-fluorophenyl)-5-(trifluoromethyl)-4,5-dihydroisoxazol-3-yl)amino)benzyl)carbamate (6 g, yield: 65%) as a yellow solid.

¹H-NMR (400 MHz, CDCl₃) δ=7.65 (dd, J=2.1, 6.8 Hz, 1H), 7.49-7.42 (m, 1H), 7.36 (br d, J=8.5 Hz, 1H), 7.27-7.24 (m, 2H), 7.20 (t, J=8.6 Hz, 1H), 6.19-6.12 (m, 1H), 5.12-5.01 (m, 1H), 4.37-4.29 (m, 2H), 3.91-3.82 (m, 1H), 3.54-3.45 (m, 1H), 1.46 (s, 9H).

Step 5: A solution of tert-butyl (2-chloro-5-((5-(3-chloro-4-fluorophenyl)-5-(trifluoromethyl)-4,5-dihydroisoxazol-3-yl)amino)benzyl)carbamate (5 g, 9.6 mmol) in HCl/EtOAc (50 mL) was stirred at 25° C. for 2 hrs. TLC (PE:EtOAc=1:1) indicated full conversion. The resulting mixture was poured into aqueous NaHCO₃ (100 mL) and extracted with EtOAc (50 mL×2). The combined organic layers were washed with brine (100 mL), dried over Na₂SO₄ and concentrated to dryness to afford the crude product, which was further purified by column chromatography on silica gel (PE:EtOAc=1:0 to 0:1) to yield N-(3-(aminomethyl)-4-chlorophenyl)-5-(3-chloro-4-fluorophenyl)-5-(trifluoromethyl)-4,5-dihydroisoxazol-3-amine (3.01 g, yield: 74.3%) as a white solid.

¹H-NMR (400 MHz, CD₃OD) δ=7.73 (dd, J=2.1, 6.9 Hz, 1H), 7.60-7.55 (m, 1H), 7.50 (d, J=1.3 Hz, 1H), 7.35 (t, J=8.9 Hz, 1H), 7.31-7.24 (m, 2H), 3.94 (d, J=16.6 Hz, 1H), 3.84 (s, 2H), 3.68 (d, J=16.6 Hz, 1H).

### Example 11: Synthesis of N-(4-(aminomethyl)-3-chlorophenyl)-5-(4-fluoro-3-((trifluoro-methyl)thio)phenyl)-5-(trifluoromethyl)-4,5-dihydroisoxazol-3-amine

To a solution of diisopropylamine (90.85 g, 704.2 mmol) in DCM/EtOAc (1333 mL/667 mL) was added dropwise diethylaminosulfur trifluoride (DAST) (124.7 g, 774.7 mmol) at −25° C.˜−10° C. and was additionally stirred for 10 min at −20˜−10° C. Then trimethyl(trifluoromethyl)silane (100 g, 704.2 mmol) was added dropwise at −25° C.˜−10° C. The reaction was stirred for 1 hr at −20˜−10° C. Then solid TsNH₂ (120.4 g, 704.23 mmol) was added at −25° C.˜−10° C. and stirred for 1 hr at −20° C., allowed to warm to 25° C. and additionally stirred for 16 hrs. TLC (PE:EtOAc=3:1) indicated full conversion. The reaction mixture was poured into H₂O (1 L), extracted with EtOAc (500 mL×2). The combined organic layers were washed with brine (500 mL), dried over Na₂SO₄ and concentrated to dryness to afford the crude product, which was further purified by column chromatography on silica gel (PE:EtOAc=1:0 to 3:1) to afford N-((diethylamino)fluoro(trifluoro-methyl)-14-sulfaneyl)-4-methylbenzenesulfonamide (220 g, yield: 91%) as brown oil.

¹H-NMR (400 MHz, CDCl₃) 5=7.76 (d, J=8.4 Hz, 2H), 7.25 (d, J=8.0 Hz, 2H), 3.36 (td, J=7.2, 14.4 Hz, 2H), 3.31-3.20 (m, 2H), 2.39 (s, 3H), 2.03 (s, 1H), 1.17 (t, J=7.3 Hz, 6H).

Step 2: To a solution of N-((diethylamino)fluoro(trifluoromethyl)-14-sulfaneyl)-4-methylbenzenesulfonamide (100 g, 290.7 mmol) in DCM (8 L) was added H₂SO₄ (94 g, 959.3 mmol) at 0° C., allowed to warm to 25° C. and additionally stirred for 2 hrs. TLC (PE:EtOAc=5:1) indicated full conversion. The reaction mixture was poured into H₂O (0.5 L), extracted with DCM (500 mL×2). The combined organic layers were washed with brine (500 mL), dried over Na₂SO₄ and concentrated to dryness to afford the crude product, 4-methyl-N-((trifluoro-methyl)thio)benzenesulfonamide, as a white solid, which was used directly without prior purification.

¹H-NMR (400 MHz, CDCl₃) 5=7.82 (d, J=8.4 Hz, 2H), 7.36 (d, J=8.1 Hz, 2H), 6.24 (br s, 1H), 2.46 (s, 3H).

Step 3: To a solution of 4-methyl-N-((trifluoromethyl)thio)benzenesulfonamide (60 g, 221.4 mmol) in DCM (600 mL) was added DIEA (31.42 g, 243.5 mmol) dropwise at 0° C. and additionally stirred for 5 min. MeOTf (38.1 g, 232.47 mmol) was added dropwise to the reaction mixture at 0˜5° C. and stirred additional 2 hrs at 0˜5° C. TLC (PE:EtOAc=5:1) indicated full conversion. The resulting reaction mixture was poured into H₂O (0.6 L), extracted with DCM (500 mL×2). The combined organic layers were washed with brine (500 mL), dried over Na₂SO₄ and concentrated to afford the crude product, which was further purified by column chromate-graphy on silica gel (PE:EtOAc=1:0 to 3:1) to yield N,4-dimethyl-N-((trifluoro-methyl)thio)benzenesulfonamide (100 g, yield: 79.2%) as white oil.

¹H-NMR (400 MHz, CDCl₃) δ=7.78 (d, J=8.4 Hz, 2H), 7.36 (d, J=8.0 Hz, 2H), 3.33 (s, 3H), 2.46 (s, 3H).

Step 4: To a solution of 2-bromo-3,3,3-trifluoroprop-1-ene (50 g, 287.4 mmol) in THF (1 L) was added B(OCH₃)₃ (36 g, 244.8 mmol) at 25° C. Then Mg (8.3 g, 344.8 mmol) was added to the mixture at 0° C. It was stirred for 4 hrs at 0˜25° C. TLC (PE:EtOAc=10:1) indicated full conversion. The resulting mixture was poured into HCl aq. (1 N), extracted with MTBE (500 mL×2). The combined organic layers were washed with brine (500 mL), dried over Na₂SO₄ and concentrated to dryness to afford (3,3,3-trifluoroprop-1-en-2-yl)boronic acid (65 g) as black oil.

¹H-NMR (400 MHz, CDCl₃) δ=6.49-6.29 (m, 2H).

Step 5: To a solution of (5-bromo-2-fluorophenyl)boronic acid (25 g, 114.16 mmol) in DME (250 mL) was added N,4-dimethyl-N-((trifluoromethyl)thio)benzenesulfonamide (42.3 g, 148.4 mmol), K₂CO₃ (31.5 g, 228.32 mmol), 4,4′-dimethylbipyridine (4.2 g, 22.83 mmol) and CuI (2.17 g, 11.42 mmol) at 25° C. and was stirred at 25° C. for 16 hrs. TLC (PE:EtOAc=10:1) indicated full conversion. The resulting reaction mixture was poured into H₂O (250 mL) and extracted with DCM (200 mL×2). The combined organic layers were washed with brine (200 mL), dried over Na₂SO₄ and concentrated to dryness to afford the crude product, which was further purified by column chromatography on silica gel (PE:EtOAc=1:0 to 5:1) to yield (5-bromo-2-fluorophe-nyl)(trifluoromethyl)sulfane (50 g, yield: 79.6%) as a colorless oil.

¹H-NMR (400 MHz, CDCl₃) δ=7.81 (dd, J=2.3, 6.2 Hz, 1H), 7.63 (ddd, J=2.6, 4.4, 8.7 Hz, 1H), 7.12 (t, J=8.4 Hz, 1H).

Step 6: To a solution of (5-bromo-2-fluorophenyl)(trifluoromethyl)sulfane (8 g, 29.09 mmol) in dioxane/H₂O (60 mL/12 mL) was added (3,3,3-trifluoroprop-1-en-2-yl)boronic acid (12.2 g, 87.27 mmol), Cs₂CO₃ (28.4 g, 87.27 mmol) and Pd(dppf)Cl₂-DCM (2.38 g, 2.9 mmol) at 25° C. and was stirred at 100° C. for 16 hrs under N₂. TLC (PE:EtOAc=10:1) indicated full conversion. The resulting reaction mixture was poured into H₂O (150 mL) and extracted with MTBE (150 mL×2).

The combined organic layers were washed with brine (200 mL), dried over Na₂SO₄ and concentrated to dryness to afford the crude product, which was further purified by column chromatography on silica gel (PE:EtOAc=1:0 to 10:1) to yield (3,3,3-trifluoroprop-1-en-2-yl)boronic acid (13.5 g, yield: 79.6%) as yellow oil.

¹H-NMR (400 MHz, CDCl₃) δ=7.96 (dd, J=2.0, 6.4 Hz, 1H), 7.82 (td, J=2.2, 4.4 Hz, 1H), 7.31 (t, J=8.5 Hz, 1H), 6.24 (d, J=1.0 Hz, 1H), 6.00 (br d, J=1.5 Hz, 1H).

Step 7: To a solution of NH(PMB)₂ (11.54 g, 44.83 mmol) in THF (80 mL) was added a solution of hydroxycarbonimidic dibromide (10.9 g, 53.79 mmol) dropwise in THF (20 mL) at −78° C. with vigorous stirring under N₂. Then diisopropylethylamine (DIEA, 5.7 g, 44.83 mmol) was added dropwise at −78° C. under N₂ within 20 min. Then a solution of (2-fluoro-5-(3,3,3-trifluoroprop-1-en-2-yl)phenyl)(trifluoromethyl)sulfane (13 g, 44.83 mmol) in THF (30 mL) added dropwise at −78° C. DIEA (11.4 g, 89.66 mmol) was added at −78° C. and allowed to warm to 20-25° C. for 16 hrs under N₂. TLC (PE:EtOAc=5:1) indicated full conversion of the starting material. The resulting solution was poured into water (200 mL) and extracted with EtOAc (2100 mL×3). The combined organic layers were washed with brine (200 mL), dried over sodium sulfate and concentrated to dryness. The residue was purified by column chromatography on silica gel (PE/EtOAc=1:0 to 3:1) to afford 5-(4-fluoro-3-((trifluoromethyl)thio)phenyl)-N,N-bis(4-methoxybenzyl)-5-(trifluoromethyl)-4,5-dihydroisoxazol-3-amine (10 g, yield: 37.9%) as yellow oil.

¹H-NMR (400 MHz, CDCl₃) δ=7.83 (dd, J=2.1, 6.3 Hz, 1H), 7.77-7.70 (m, 1H), 7.27-7.24 (m, 1H), 7.07 (d, J=8.6 Hz, 4H), 6.87-6.83 (m, 4H), 4.29-4.26 (m, 4H), 3.83 (br d, J=6.0 Hz, 1H), 3.81-3.80 (m, 6H), 3.42 (d, J=16.0 Hz, 1H).

Step 8: A solution of 5-(4-fluoro-3-((trifluoromethyl)thio)phenyl)-N,N-bis(4-methoxybenzyl)-5-(trifluoromethyl)-4,5-dihydroisoxazol-3-amine (10 g, 17.0 mmol) in TFA (20 mL) was stirred at 25° C. for 16 hrs. TLC (PE:EtOAc=3:1) indicated full conversion. The resulting mixture was quenched with NaHCO₃ aq. (800 mL), extracted with EtOAc (200 mL×3), the combined organic layers were washed with brine (200 mL), dried over Na₂SO₄ and concentrated to dryness to afford the crude product, which was further purified by column chromatography on silica gel (PE/EtOAc=1:0 to 0:1) to yield 5-(4-fluoro-3-((trifluoromethyl)thio)phenyl)-5-(trifluoromethyl)-4,5-dihydroisoxazol-3-amine (5 g, yield: 84.47%) as yellow oil.

¹H-NMR (400 MHz, CDCl₃) δ=7.83 (dd, J=2.0, 6.4 Hz, 1H), 7.77-7.71 (m, 1H), 7.30-7.25 (m, 1H), 3.74 (d, J=16.4 Hz, 1H), 3.37 (d, J=16.4 Hz, 1H).

Step 9: To a solution of 5-(4-fluoro-3-((trifluoromethyl)thio)phenyl)-5-(trifluoromethyl)-4,5-dihydroisoxazol-3-amine (10 g, 28.74 mmol) and tert-butyl (5-bromo-2-chlorobenzyl)carbamate (13.75 g, 43.1 mmol) in dioxane (100 mL) was added Pd₂(dba)₃ (2.6 g, 2.87 mmol), Xantphos (3.3 g, 5.75 mmol) and K₃PO₄ (18.27 g, 86.19 mmol) and was stirred at 110° C. for 16 hrs under N₂. TLC (PE:EtOAc=3:1) indicated full conversion. The resulting mixture was poured into H₂O (200 mL), extracted with EtOAc (100 mL×2). The combined organic layers were washed with brine (100 mL), dried over Na₂SO₄ and concentrated to dryness to afford the crude product, which was further purified by column chromatography (PE:EtOAc=5:1 to 0:1) to yield tert-butyl (2-chloro-5-((5-(4-fluoro-3-((trifluoromethyl)thio)phenyl)-5-(trifluoromethyl)-4,5-dihydroisoxazol-3-yl)amino)benzyl)carbamate (7 g, yield: 41.4%) as a yellow solid.

¹H-NMR (400 MHz, CDCl₃) δ=7.88-7.83 (m, 1H), 7.79-7.72 (m, 1H), 7.36 (br d, J=8.4 Hz, 1H), 7.31-7.27 (m, 2H), 7.25 (br d, J=6.0 Hz, 1H), 6.21 (s, 1H), 5.07 (br s, 1H), 4.33 (d, J=6.4 Hz, 2H), 3.89 (d, J=16.0 Hz, 1H), 3.52 (d, J=16.1 Hz, 1H), 1.46 (s, 9H).

Step 10: A solution of tert-butyl (2-chloro-5-((5-(4-fluoro-3-((trifluoromethyl)thio)phenyl)-5-(tri-fluoromethyl)-4,5-dihydroisoxazol-3-yl)amino)benzyl)carbamate (7 g, 11.9 mmol) in HCl/EtOAc (70 mL) was stirred at 25° C. for 2 hrs. TLC (PE:EtOAc=1:1) indicated full conversion. The resulting mixture was poured into NaHCO₃ (1 L) and extracted with EtOAc (200 mL×2). The combined organic layers were washed with brine (200 mL), dried over Na₂SO₄ and concentrated to dryness to afford the crude product, which was further purified by prep-HPLC (H₂O-MeCN, TFA) to yield N-(3-(aminomethyl)-4-chlorophenyl)-5-(4-fluoro-3-((trifluoromethyl)thio)phenyl)-5-(trifluoromethyl)-4,5-dihydroisoxazol-3-amine (2.29 g, yield: 39.5%) as a white solid.

¹H-NMR (400 MHz, CD₃OD) δ=7.96 (dd, J=1.9, 6.4 Hz, 1H), 7.90-7.84 (m, 1H), 7.51 (d, J=1.4 Hz, 1H), 7.44 (t, J=8.5 Hz, 1H), 7.32-7.24 (m, 2H), 3.98 (d, J=16.6 Hz, 1H), 3.85 (s, 2H), 3.71 (d, J=16.8 Hz, 1H).

### Example 12: ethyl (3-(cyclopropanecarboxamidomethyl)-4-fluorophenyl)(5-(3,5-dichloro-4-fluorophenyl)-5-(trifluoromethyl)-4,5-dihydroisoxazol-3-yl)carbamate

To a solution of ethyl (3-(aminomethyl)-4-fluorophenyl)(5-(3,5-dichloro-4-fluorophenyl)-5-(tri-fluoromethyl)-4,5-dihydroisoxazol-3-yl)carbamate (250 mg, 0.49 mmol) in ethyl acetate (3.8 mL) was added cyclopropanecarboxylic acid (46.2 mg, 0.54 mmol), a solution of propanephosphonic acid anhydride in EtOAc (50%, 1.41 ml, 2.44 mmol) and pyridine (0.20 ml, 2.44 mmol). The reaction was stirred at 20° C. until TLC indicated full conversion. All volatile compounds were evaporated under reduced pressure followed by column chromatography on silica gel (cyclohexane/EtOAc; gradient 0 to 100% EtOAc) to yield the title compound as colorless solid (250 mg, 51%).

¹H-NMR (500 MHz, MeOD) δ 7.66 (d, J=6.1 Hz, 2H), 7.24 (dd, J=6.6, 2.6 Hz, 1H), 7.22-7.18 (m, 1H), 7.18-7.13 (m, 1H), 4.85 (s, 2H), 4.47 (d, J=18.4 Hz, 1H), 4.43 (sbr, 2H), 4.20-4.14 (m, 3H), 1.60 (tt, J=8.0, 4.7 Hz, 1H), 1.16 (t, J=7.1 Hz, 3H), 0.84 (dt, J=4.5, 3.0 Hz, 2H), 0.75 (dt, J=8.0, 3.2 Hz, 2H).

### Example 13: N-(2-chloro-5-((5-(3,5-dichloro-4-fluorophenyl)-5-(trifluoromethyl)-4,5-dihydro-isoxazol-3-yl)amino)benzyl)cyclopropanecarboxamide

To a solution of N-(3-(aminomethyl)-4-chlorophenyl)-5-(3,5-dichloro-4-fluorophenyl)-5-(tri-fluoromethyl)-4,5-dihydroisoxazol-3-amine (400 mg, 0.88 mmol) in dichloromethane (10 mL) was added cyclopropanecarboxylic acid (75.4 mg, 0.88 mmol), a solution of propanephosphonic acid anhydride in EtOAc (50%, 3.34 g, 5.26 mmol) and 4-dimethylaminopyridine (321 mg, 2.63 mmol). The reaction was stirred for 16 h at 20° C. TLC indicated full conversion. The reaction was quenched by addition of water, followed by extraction with dichloromethane. The combined organic phase was dried over sodium sulfate and concentrated to dryness to afford the crude product, which was further purified by flash column chromatography on silica gel (cyclohexane/EtOAc; gradient 0 to 60% EtOAc) to afford the title product as colorless solid (300 mg, 65%).

¹H-NMR (400 MHz, MeOD) δ 7.70 (d, J=6.2 Hz, 2H), 7.42 (d, J=2.5 Hz, 1H), 7.34-7.26 (m, 2H), 4.88 (sbr, 1H), 4.42 (s, 2H), 3.93 (d, J=16.7 Hz, 1H), 3.67 (d, J=16.7 Hz, 1H), 1.69 (tt, J=8.0, 4.7 Hz, 1H), 0.91 (dt, J=4.7, 3.1 Hz, 2H), 0.81 (dt, J=8.0, 3.1 Hz, 2H).

### Example 14: N-(2-chloro-5-((5-(3-chloro-4-fluorophenyl)-5-(trifluoromethyl)-4,5-dihydro-isoxazol-3-yl)amino)benzyl)cyclopropanecarboxamide

To a solution of N-(3-(aminomethyl)-4-chlorophenyl)-5-(3-chloro-4-fluorophenyl)-5-(trifluoro-methyl)-4,5-dihydroisoxazol-3-amine (200 mg, 0.47 mmol) in ethyl acetate (3.9 mL) was added cyclopropanecarboxylic acid (44.9 mg, 0.52 mmol), a solution of propanephosphonic acid anhydride in EtOAc (50%, 1.37 mL, 2.37 mmol) and pyridine (0.19 mL, 2.37 mmol). The reaction was stirred for 16 h at 20° C. TLC analysis indicated full conversion. The reaction was quenched by addition of water, followed by extraction with dichloromethane. The combined organic phase was dried over sodium sulfate and concentrated to dryness to afford the crude product, which was further purified by flash column chromatography on silica gel (cyclohexane/EtOAc; gradient 0 to 100% EtOAc) to afford the title product as colorless solid (98 mg, 42%).

¹H-NMR (500 MHz, MeOD) δ 7.72 (dd, J=6.9, 2.3 Hz, 1H), 7.56 (ddd, J=8.8, 4.4, 2.3 Hz, 1H), 7.42 (d, J=2.7 Hz, 1H), 7.36-7.25 (m, 3H), 4.86 (sbr, 2H), 4.42 (s, 2H), 3.92 (d, J=16.6 Hz, 1H), 3.65 (d, J=16.6 Hz, 1H), 1.69 (tt, J=8.0, 4.6 Hz, 1H), 0.91 (dt, J=4.7, 3.3 Hz, 2H), 0.81 (dt, J=8.1, 3.3 Hz, 2H).

### Example 15: N-(2-chloro-5-((5-(4-fluoro-3-((trifluoromethyl)thio)phenyl)-5-(trifluoromethyl)-4,5-dihydroisoxazol-3-yl)amino)benzyl)cyclopropanecarboxamide

To a solution of N-(3-(aminomethyl)-4-chlorophenyl)-5-(4-fluoro-3-((trifluoromethyl)thio)phenyl)-5-(trifluoromethyl)-4,5-dihydroisoxazol-3-amine (250 mg, 0.51 mmol) in pyridine (3.9 mL) was added cyclopropanecarboxylic acid (48.5 mg, 0.56 mmol), a solution of propanephosphonic acid anhydride in EtOAc (50%, 1.78 mL, 3.07 mmol) and 4-dimethylaminopyridine (63.0 mg, 0.51 mmol). The reaction was stirred for 16 h at 20° C. TLC analysis indicated full conversion. The reaction was quenched by addition of water, followed by extraction with dichloromethane. The combined organic phase was dried over sodium sulfate and concentrated to dryness to afford the crude product, which was further purified by flash column chromatography on silica gel (cyclohexane/EtOAc; gradient 0 to 100% EtOAc) to afford the title product as colorless solid (134 mg, 47%).

¹H-NMR (400 MHz, MeOD) δ 8.51 (t, J=5.9 Hz, 1H), 7.95 (dd, J=6.6, 2.4 Hz, 1H), 7.88-7.81 (m, 1H), 7.44-7.37 (m, 2H), 7.31-7.20 (m, 2H), 4.86 (s, 1H), 4.42-4.38 (s, 2H), 3.95 (d, J=16.6 Hz, 1H), 3.67 (d, J=16.6 Hz, 1H), 1.69 (tt, J=8.3, 4.6 Hz, 1H), 0.90 (dt, J=6.2, 3.2 Hz, 2H), 0.80 (dt, J=8.1, 3.3 Hz, 2H).

### Example 16: N-(5-((5-(3-chloro-4-fluorophenyl)-5-(dichloromethyl)-4,5-dihydroisoxazol-3-yl)amino)-2-methoxybenzyl)acetamide

To a solution of N-(3-(aminomethyl)-4-methoxyphenyl)-5-(3-chloro-4-fluorophenyl)-5-(dichloro-methyl)-4,5-dihydroisoxazol-3-amine (459 mg, 1.06 mmol) in THF (16 mL) was added acetic anhydride (130 mg, 1.27 mmol), diisopropylethylamine (822 mg, 6.37 mmol) and 4-dimethylaminopyridine (13.0 mg, 0.11 mmol). The reaction was stirred for 20 h at 20° C. LC-MS indicated full conversion. The reaction was quenched by addition of water, followed by extraction with dichloromethane. The combined organic phase was dried over sodium sulfate and concentrated to dryness to afford the crude product, which was further purified by flash column chromate-graphy on silica gel (cyclohexane/EtOAc; gradient 0 to 100% EtOAc) to afford the title product as colorless solid (373 mg, 74%).

¹H-NMR (400 MHz, MeOD) δ 7.73 (dd, J=7.0, 2.3 Hz, 1H), 7.56 (ddd, J=8.7, 4.5, 2.4 Hz, 1H), 7.32-7.24 (m, 2H), 7.20 (d, J=2.8 Hz, 1H), 6.87 (d, J=8.8 Hz, 1H), 6.39 (s, 1H), 4.84 (sbr, 2H), 4.31 (s, 2H), 3.89 (d, J=16.4 Hz, 1H), 3.80 (s, 3H), 3.54 (d, J=16.4 Hz, 1H), 1.99 (s, 3H).

### Example 17: N-((4-((5-(3-chloro-4-fluorophenyl)-5-(dichloromethyl)-4,5-dihydroisoxazol-3-yl)amino)pyridin-2-yl)methyl)cyclopropanecarboxamide

To a solution of N-(2-(aminomethyl)pyridin-4-yl)-5-(3-chloro-4-fluorophenyl)-5-(dichloromethyl)-4,5-dihydroisoxazol-3-amine (90 mg, 0.22 mmol) in THF (4 mL) was added cyclopropanecarboxylic acid anhydride (41.3 mg, 0.27 mmol), diisopropylethylamine (173 mg, 1.34 mmol) and 4-dimethylaminopyridine (2.72 mg, 0.02 mmol). The reaction was stirred for 18 h at 20° C. LC-MS indicated full conversion. The reaction was quenched by addition of water, followed by extraction with dichloromethane. The combined organic phase was dried over sodium sulfate and concentrated to dryness to afford the crude product, which was further purified by flash column chromatography on silica gel (cyclohexane/EtOAc; gradient 0 to 100% EtOAc) to afford the title product as colorless solid (72 mg, 68%).

¹H-NMR (500 MHz, CDCl₃) δ 8.60 (s, 1H), 8.29 (d, J=5.7 Hz, 1H), 7.72 (dd, J=6.9, 2.3 Hz, 1H), 7.56-7.46 (m, 1H), 7.24 (t, J=8.6 Hz, 1H), 7.17 (d, J=2.2 Hz, 1H), 6.03 (s, 1H), 5.37 (s, OH), 4.51 (dd, J=5.4, 2.3 Hz, 2H), 3.94 (d, J=16.6 Hz, 1H), 3.61 (d, J=16.6 Hz, 1H), 2.43 (s, 1H), 1.66 (tt, J=8.1, 4.6 Hz, 1H), 1.06-0.97 (m, 2H), 0.97-0.85 (m, 2H).

TABLE I.1

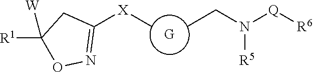

compounds of formula I (Y = CH₂)

| | | | | | | | | | phys. data | |
|---|---|---|---|---|---|---|---|---|---|---|
| | | | | | | | | | [HPLC Rt [min], | HPLC |
| No. | W | R¹ | X | R³ | G | R⁵ | Q | R⁶ | M + H [m/z]] | method |
| I-1 | W-9 | CF₃ | NR³ | H | 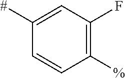 | H | C(═O) | C₂H₅ | 1.277, 478.0 | A |
| I-2 | W-9 | CF₃ | NR³ | H | G1, R⁴¹: F, R⁴²: H | H | C(═O) | C₂H₅ | 1.268, 478.0 | A |
| I-3 | W-9 | CF₃ | NR³ | H | G1, R⁴¹: F, R⁴²: H | H | C(═O) | (CH₂)₂OCH₃ | 1.256, 508.3 | A |
| I-4 | W-9 | CF₃ | NR³ | H | G1, R⁴¹: F, R⁴²: H | H | C(═O) | CH₂S(O)₂CH₃ | 1.216, 542.3 | A |
| I-5 | W-9 | CF₃ | NR³ | H | G1, R⁴¹: F, R⁴²: H | H | C(═O) | 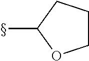 | 1.299, 520.3 | A |
| I-6 | W-9 | CF₃ | NR³ | H | G1, R⁴¹: F, R⁴²: H | H | C(═O) | CH₂—cC₃H₅ | 1.304, 504.0 | A |
| I-7 | W-9 | CF₃ | NR³ | H | G1, R⁴¹: F, R⁴²: H | H | C(═O) | 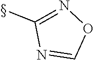 | 1.257, 518.3 | A |
| I-8 | W-9 | CF₃ | NR³ | H | G1, R⁴¹: F, R⁴²: H | H | C(═O) | c-C₃H₅ | 1.292, 490.3 | A |
| I-9 | W-9 | CF₃ | NR³ | H | G1, R⁴¹: F, R⁴²: H | H | C(═O) | CH₂OCH₃ | 1.269, 494.3 | A |
| I-10 | W-9 | CF₃ | NR³ | H | G1, R⁴¹: F, R⁴²: H | H | C(═O) | 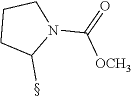 | 1.263, 577.0 | A |
| I-11 | W-9 | CF₃ | NR³ | H | G1, R⁴¹: F, R⁴²: H | H | C(═O) | 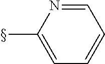 | 1.324, 527.3 | A |
| I-12 | W-9 | CF₃ | NR³ | H | G1, R⁴¹: F, R⁴²: H | H | C(═O) | CH(═NOCH₃) | 1.293, 506.9 | A |
| I-13 | W-9 | CF₃ | NR³ | H | G1, R⁴¹: F, R⁴²: H | H | S(O)₂ | C₂H₅ | 1.258, 514.3 | A |
| I-14 | W-11 | CF₃ | NR³ | H | G1, R⁴¹: Cl, R⁴²: H | H | C(═O) | cC₃H₅ | 1.320, 526.1 | A |
| I-15 | W-11 | CF₃ | NR³ | C₂H₅ | G1, R⁴¹: F, R⁴²: H | H | C(═O) | cC₃H₅ | 1.342, 536.2 | A |
| I-16 | W-11 | CF₃ | NR³ | H | G1, R⁴¹: F, R⁴²: H | H | C(═O) | cC₃H₅ | 1.276, 508.2 | A |
| I-17 | W-11 | CF₃ | NR³ | H | G1, R⁴¹: F, R⁴²: H | CH₃ | C(═O) | cC₃H₅ | 1.345, 522.2 | A |
| I-18 | W-11 | CF₃ | NR³ | H | G1, R⁴¹: OCF₃, R⁴²: H | H | C(═O) | cC₃H₅ | 1.357, 574.1 | A |
| I-19 | W-11 | CF₃ | NR³ | CH₂OC₂H₅ | G1, R⁴¹: F, R⁴²: H | H | C(═O) | cC₃H₅ | 1.385, 520.2 | A |
| I-20 | W-11 | CF₃ | NR³ | H | G1, R⁴¹: CH₃, R⁴²: H | H | C(═O) | cC₃H₅ | 1.324, 503.9 | A |
| I-21 | W-8 | CF₃ | NR³ | H | G1, R⁴¹: Cl, R⁴²: H | H | C(═O) | cC₃H₅ | 1.277, 489.8 | A |
| I-22 | W-11 | CF₃ | O | — | G1, R⁴¹: F, R⁴²: H | H | C(═O) | cC₃H₅ | 1.353, 508.8 | A |
| I-23 | W-8 | CF₃ | NR³ | H | G1, R⁴¹: Cl, R⁴²: H | H | C(═O) | cC₄H₇ | 1.319, 503.9 | A |
| I-24 | W-8 | CF₃ | NR³ | H | G1, R⁴¹: Cl, R⁴²: H | H | C(═O) | (1-SO₂CH₃)-cC₃H₄ | 1.265, 568.1 | A |
| I-25 | W-8 | CF₃ | NR³ | H | G1, R⁴¹: Cl, R⁴²: H | H | C(═O) | i-C₃H₇ | 1.299, 491.9 | A |
| I-26 | W-8 | CF₃ | NR³ | H | G1, R⁴¹: Cl, R⁴²: H | H | C(═O) | 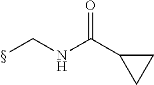 | 1.209, 547.2 | A |
| I-27 | W-8 | CF₃ | NR³ | H | G1, R⁴¹: Cl, R⁴²: H | H | C(═O) | CH₂C₆H₅ | 1.343, 539.9 | A |
| I-28 | W-8 | CF₃ | NR³ | H | G1, R⁴¹: Cl, R⁴²: H | H | C(═O) | i-C₄H₉ | 1.314, 506.1 | A |
| I-29 | W-8 | CF₃ | NR³ | H | G1, R⁴¹: Cl, R⁴²: H | H | C(═O) | 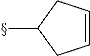 | 1.313, 516.1 | A |
| I-30 | W-8 | CF₃ | NR³ | H | G1, R⁴¹: Cl, R⁴²: H | H | C(═O) | 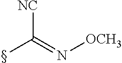 | 1.315, 532.1 | A |
| I-31 | W-8 | CF₃ | NR³ | H | G1, R⁴¹: Cl, R⁴²: H | H | C(═O) | 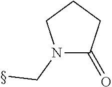 | 1.179, 547.1 | A |
| I-32 | W-8 | CF₃ | NR³ | H | G1, R⁴¹: Cl, R⁴²: H | H | C(═O) | 1-CN-cC₃H₄ | 1.258, 515.1 | A |
| I-33 | W-8 | CF₃ | NR³ | H | G1, R⁴¹: Cl, R⁴²: H | H | C(═O) | 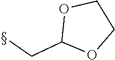 | 1.238, 535.9 | A |
| I-34 | W-8 | CF₃ | NR³ | H | G1, R⁴¹: Cl, R⁴²: H | H | C(═O) | 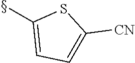 | 1.318, 579.1 | A |
| I-35 | W-8 | CF₃ | NR³ | H | G1, R⁴¹: Cl, R⁴²: H | H | C(═O) | 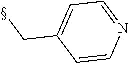 | 1.034, 541.1 | A |
| I-36 | W-8 | CF₃ | NR³ | H | G1, R⁴¹: Cl, R⁴²: H | H | C(═O) | 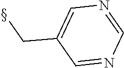 | 1.174, 542.1 | A |
| I-37 | W-8 | CF₃ | NR³ | H | G1, R⁴¹: Cl, R⁴²: H | H | C(═O) | 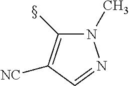 | 1.274, 577.1 | A |
| I-38 | W-8 | CF₃ | NR³ | H | G1, R⁴¹: Cl, R⁴²: H | H | C(═O) | 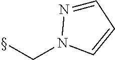 | 1.229, 552.1 | A |
| I-39 | W-8 | CF₃ | NR³ | H | G1, R⁴¹: Cl, R⁴²: H | H | C(═O) | 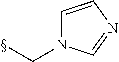 | 1.309, 530.1 | A |
| I-40 | W-8 | CF₃ | NR³ | H | G1, R⁴¹: Cl, R⁴²: H | H | C(═O) | 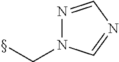 | 1.154, 531.1 | A |
| I-41 | W-8 | CF₃ | NR³ | H | G1, R⁴¹: Cl, R⁴²: H | H | C(═O) | 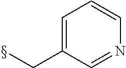 | 1.036, 541.1 | A |
| I-42 | W-11 | CF₃ | NR³ | C(═O)OC₂H₅ | G1, R⁴¹: F, R⁴²: H | H | C(═O) | cC₃H₅ | 1.384, 579.9 | A |
| I-43 | W-9 | CF₂Cl | NR³ | H | G1, R⁴¹: F, R⁴²: H | H | C(═O) | cC₃H₅ | 1.321, 506.1 | A |
| I-44 | W-8 | CF₃ | NR³ | H | G1, R⁴¹: Cl, R⁴²: H | H | C(═O) | 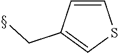 | 1.302, 546.1 | A |
| I-45 | W-8 | CF₃ | NR³ | H | G1, R⁴¹: Cl, R⁴²: H | H | C(═O) | 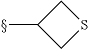 | 1.276, 522.0 | A |
| I-46 | W-8 | CF₃ | NR³ | H | G1, R⁴¹: Cl, R⁴²: H | H | C(═O) | 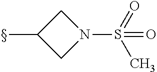 | 1.189, 583.1 | A |
| I-47 | W-8 | CF₃ | NR³ | H | G1, R⁴¹: Cl, R⁴²: H | H | C(═O) | CH₂NHSO₂CH₃ | 1.164, 557.0 | A |
| I-48 | W-8 | CF₃ | NR³ | H | G1, R⁴¹: Cl, R⁴²: H | H | C(═O) | 2,2-F₂-cC₃H₃ | 1.273, 526.0 | A |
| I-49 | W-8 | CF₃ | NR³ | H | G1, R⁴¹: Cl, R⁴²: H | H | C(═O) | 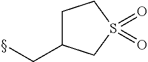 | 1.169, 582.1 | A |
| I-50 | W-8 | CF₃ | NR³ | H | G1, R⁴¹: Cl, R⁴²: H | H | C(═O) | C₂H₅ | 1.234, 478.0 | A |
| I-51 | W-8 | CF₃ | NR³ | H | G1, R⁴¹: Cl, R⁴²: H | H | C(═O) | CH₂CN | 1.203, 489.1 | A |
| I-52 | W-8 | CF₃ | NR³ | H | G1, R⁴¹: Cl, R⁴²: H | H | C(═O) | 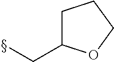 | 1.254, 534.1 | A |
| I-53 | W-8 | CF₃ | NR³ | H | G1, R⁴¹: Cl, R⁴²: H | H | C(═O) | 2,2-Cl₂-cC₃H₃ | 1.327, 559.9 | A |
| I-54 | W-8 | CF₃ | NR³ | H | G1, R⁴¹: Cl, R⁴²: H | H | C(═O) | 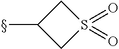 | 1.196, 554.0 | A |
| I-55 | W-8 | CF₃ | NR³ | H | G1, R⁴¹: Cl, R⁴²: H | H | C(═O) | CH₂CH₂CN | 1.201, 503.1 | A |
| I-56 | W-8 | CF₃ | NR³ | H | G1, R⁴¹: Cl, R⁴²: H | H | C(═O) | 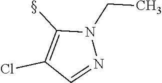 | 1.371, 580.0 | A |
| I-57 | W-8 | CF₃ | NR³ | H | G1, R⁴¹: Cl, R⁴²: H | H | C(═O) | 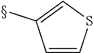 | 1.294, 532.0 | A |
| I-58 | W-8 | CF₃ | NR³ | H | G1, R⁴¹: Cl, R⁴²: H | H | C(═O) | H | 1.187, 450.0 | A |
| I-59 | W-8 | CF₃ | NR³ | H | G1, R⁴¹: Cl, R⁴²: H | H | C(═O) | CH₃ | 1.189, 464.0 | A |
| I-60 | W-34 | CF₃ | NR³ | H | G1, R⁴¹: Cl, R⁴²: H | H | C(═O) | cC₃H₅ | 1.330, 555.9 | A |
| I-61 | W-8 | CHCl₂ | NR³ | H | G1, R⁴¹: Cl, R⁴²: H | H | C(═O) | CH₃ | 1.173, 479.8 | A |
| I-62 | W-8 | CHCl₂ | NR³ | H | G1, R⁴¹: Cl, R⁴²: H | H | C(═O) | cC₃H₅ | 1.236, 505.9 | A |
| I-63 | W-8 | CHCl₂ | NR³ | H | G1, R⁴¹: Cl, R⁴²: H | H | C(═O) | C₂H₅ | 1.221, 493.8 | A |
| I-64 | W-33 | CHCl₂ | NR³ | H | G1, R⁴¹: Cl, R⁴²: H | H | C(═O) | cC₃H₅ | 1.286, 521.8 | A |
| I-65 | W-33 | CHCl₂ | NR³ | H | G1, R⁴¹: Cl, R⁴²: H | H | C(═O) | CH₃ | 1.227, 495.7 | A |
| I-66 | W-33 | CHCl₂ | NR³ | H | G1, R⁴¹: Cl, R⁴²: H | H | C(═O) | C₂H₅ | 1.267, 509.8 | A |
| I-67 | W-8 | CHCl₂ | NR³ | H | G1, R⁴¹: OCH₃, R⁴²: H | H | C(═O) | cC₃H₅ | 1.188, 501.9 | A |
| I-68 | W-8 | CHCl₂ | NR³ | H | G1, R⁴¹: OCH₃, R⁴²: H | H | C(═O) | CH₃ | 1.129, 473.9 | A |
| I-69 | W-8 | CHCl₂ | NR³ | H | G1, R⁴¹: OCH₃, R⁴²: H | H | C(═O) | C₂H₅ | 1.173, 489.9 | A |
| I-70 | W-8 | CClF₂ | NR³ | C₂H₅ | G1, R⁴¹: Cl, R⁴²: H | H | C(═O) | cC₃H₅ | 1.343, 535.8 | A |
| I-71 | W-8 | CClF₂ | NR³ | C₂H₅ | G1, R⁴¹: Cl, R⁴²: H | H | C(═O) | CH₃ | 1.277, 509.8 | A |
| I-72 | W-8 | CClF₂ | NR³ | C₂H₅ | G1, R⁴¹: Cl, R⁴²: H | H | C(═O) | C₂H₅ | 1.322, 523.9 | A |
| I-73 | W-9 | CClF₂ | NR³ | H | G1, R⁴¹: Cl, R⁴²: H | H | C(═O) | C₂H₅ | 1.322, 511.7 | A |
| I-74 | W-9 | CClF₂ | NR³ | H | G1, R⁴¹: Cl, R⁴²: H | H | C(═O) | CH₃ | 1.301, 497.9 | A |
| I-75 | W-8 | CHCl₂ | NR³ | C₂H₅ | G1, R⁴¹: Cl, R⁴²: H | H | C(═O) | CH₃ | 1.247, 507.9 | A |
| I-76 | W-8 | CHCl₂ | NR³ | C₂H₅ | G1, R⁴¹: Cl, R⁴²: H | H | C(═O) | C₂H₅ | 1.341, 519.9 | A |
| I-77 | W-8 | CHCl₂ | NR³ | C₂H₅ | G1, R⁴¹: OCH₃, R⁴²: H | H | C(═O) | CH3 | 1.200, 503.9 | A |
| I-78 | W-9 | CClF₂ | NR³ | H | G1, R⁴¹: Cl, R⁴²: H | H | C(═O) | cC₃H₅ | 1.338, 523.8 | A |
| I-79 | W-9 | CHCl₂ | NR³ | H | G1, R⁴¹: Cl, R⁴²: H | H | C(═O) | C₂H₅ | 1.291, 509.7 | A |
| I-80 | W-9 | CHCl₂ | NR³ | H | G1, R⁴¹: Cl, R⁴²: H | H | C(═O) | CH₃ | 1.250, 495.7 | A |
| I-81 | W-9 | CHCl₂ | NR³ | H | G1, R⁴¹: Cl, R⁴²: H | H | C(═O) | 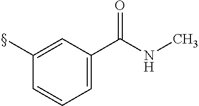 | 1.280, 614.9 | A |
| I-82 | W-9 | CHCl₂ | NR³ | H | G1, R⁴¹: Cl, R⁴²: H | H | C(═O) | cC₃H₅ | 1.360, 583.7 | A |
| I-83 | W-8 | CHCl₂ | NR³ | H | G1, R⁴¹: OCH₃, R⁴²: H | H | C(═O) | C₂H₅ | 1.259, 517.9 | A |
| I-84 | W-9 | CHCl₂ | NR³ | H | G1, R⁴¹: Cl, R⁴²: H | H | C(═O) | 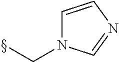 | 1.104, 561.9 | A |
| I-85 | W-8 | CHCl₂ | NR³ | H | G4, R⁴²: H | H | C(═O) | cC₃H₅ | cf. Ex. 17 | |

§ = bond to the remainder of the molecule; # = bond to X; % = bond to Y

TABLE I.2

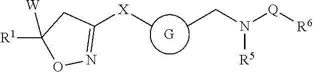

compounds of formula I (Y = direct bond)

| | | | | | | | | | phys. data | |
|---|---|---|---|---|---|---|---|---|---|---|
| | | | | | | | | | [HPLC Rt [min], | HPLC |
| No | W | R¹ | X | R³ | G | R⁵ | Q | R⁶ | M + H [m/z]] | method |
| II-1 | W-9 | CF₃ | NR³ | C(O)OC(CH₃)₃ | G1, R⁴¹: Cl, R⁴²: H | H | C(═O) | CH₂S(O)₂CH₃ | 1.398, 545.8 | A |
| II-2 | W-9 | CF₃ | NR³ | H | G1, R⁴¹: Cl, R⁴²: H | H | C(═O) | CH₂S(O)₂CH₃ | 1.252, 546.0 | A |

§ = bond to the remainder of the molecule; # = bond to X; % = bond to Y

TABLE I.3

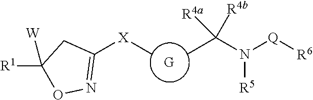

compounds of formula 1, wherein R⁴ forms together with R^{4a}/R^{4b} a ring (Y = CR^{4a}R^{4b})

| | | | | | | | | | | | |
|---|---|---|---|---|---|---|---|---|---|---|---|
| | | | | | | | | | | phys. data | |
| | | | | | | | | | | [HPLC Rt [min], | HPLC |
| No. | W | R¹ | X | R³ | G | R⁴ + R^{4a}/R^{4b} | R⁵ | Q | R⁶ | M + H [m/z]] | method |

| | | | | | | | | | | |
|---|---|---|---|---|---|---|---|---|---|---|
| III-1 | W-8 | CF₃ | NR³ | H | 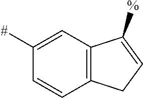 | H | C(═O) | cC₃H₅ | 1.242, 481.9 | A |
| III-2 | W-8 | CF₃ | NR³ | H | 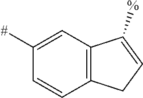 | H | C(═O) | cC₃H₅ | 1.235, 482.2 | A |

§ = bond to the remainder of the molecule; # = bond to X; % = bond to Y

### Biological Examples

If not otherwise specified, the test solutions were prepared as follows:

The active compound was dissolved at the desired concentration in a mixture of 1:1 (vol:vol) distilled water:acetone. The test solution was prepared at the day of use.

Test solutions were prepared in general at a concentration of 2500 ppm (wt/vol).

B.1. Boll weevil (Anthonomus grandis)

For evaluating control of boll weevil, the test unit consisted of 96-well-microtiter plates containing an insect diet and 5-10 A. grandis eggs.

The compounds were formulated using a solution containing 75% v/v water and 25% v/v DMSO. Different concentrations of formulated compounds were sprayed onto the insect diet at 5 μl, using a custom-built micro atomizer, at two replications.

After application, microtiter plates were incubated at about 25±1° C. and about 75±5% relative humidity for 5 days. Egg and larval mortality were then visually assessed.

In this test, compounds I-2, I-3, I-4, I-6, I-7, I-8, I-9, I-10, I-11, I-12, I-13, I-14, I-15, I-16, I-17, I-18, I-19, I-20, I-21, I-22, I-23, I-25, I-26, I-28, I-29, I-32, I-33, I-34, I-38, I-40, I-42, I-43, I-44, I-45, I-46, I-47, I-48, I-49, I-50, I-51, I-52, I-53, I-54, I-55, I-58, I-59, I-60, I-61, I-62, I-63, I-64, I-65, I-66, I-69, I-70, I-71, I-72, I-75, I-76, I-78, I-79, I-80, I-81, I-82, I-84, and III-1, resp., at 800 ppm showed at least 75% mortality in comparison with untreated controls.

B.2. Green Peach Aphid (Myzus persicae) (Mixed Life Stages)

For evaluating control of Green Peach Aphid through systemic means the test unit consisted of 96-well-microtiter plates containing liquid artificial diet under an artificial membrane. The compounds were formulated using a solution containing 75% v/v water and 25% v/v DMSO. Different concentrations of formulated compounds were pipetted into the aphid diet, using a custom-built pipette, at two replications. After application, 5-8 adult aphids were placed on the artificial membrane inside the microtiter plate wells. The aphids were then allowed to suck on the treated aphid diet and incubated at about 23±1° C. and about 50±5% relative humidity for 3 days. Aphid mortality and fecundity was then visually assessed.

In this test, compounds I-2, I-3, I-4, I-5, I-6, I-8, I-9, I-12, I-13, I-14, I-15, I-16, I-18, I-19, I-20, I-21, I-22, I-23, I-25, I-26, I-28, I-29, I-31, I-32, I-40, I-42, I-43, I-44, I-45, I-46, I-47, I-48, I-49, I-50, I-51, I-52, I-53, I-54, I-55, I-58, I-59, I-60, I-61, I-62, I-63, I-64, I-65, I-66, I-67, I-68, I-69, I-70, I-71, I-72, I-73, I-74, I-77, I-78, I-79, I-80, I-82 I-83, and III-1, resp., at 800 ppm showed at least 75% mortality in comparison with untreated controls.

B.3. Tobacco Budworm (Heliothis virescens)

For evaluating control of Tobacco Budworm, the test unit consisted of 96-well-microtiter plates containing an insect diet and 15-25 H. virescens eggs. The compounds were formulated using a solution containing 75% v/v water and 25% v/v DMSO. Different concentrations of formulated compounds were sprayed onto the insect diet at 10 μl, using a custom-built micro atomizer, at two replications. After application, microtiter plates were incubated at about 28±1° C. and about 80±5% relative humidity for 5 days. Egg and larval mortality was then visually assessed.

In this test, compounds I-1, I-2, I-3, I-4, I-6, I-8, I-9, I-12, I-13, I-14, I-15, I-16, I-17, I-18, I-19, I-20, I-21, I-22, I-42, I-73, I-74, I-78, I-79, I-80, I-82, and I-84, resp., at 2500 ppm showed at least 75% mortality in comparison with untreated controls.

In this test, compounds I-2, I-3, I-4, I-6, I-8, I-12, I-13, I-14, I-15, I-16, I-17, I-18, I-19, I-20, I-21, I-22, I-23, I-25, I-28, I-32, I-42, I-43, I-45, I-48, I-50, I-53, I-54, I-55, I-59, I-60, I-62, I-64, I-66, I-70, I-72, and III-1, resp., at 800 ppm showed at least 75% mortality in comparison with untreated controls.

B.4. Yellow Fever Mosquito (Aedes aegypti)

For evaluating control of Yellow Fever Mosquito, the test unit consisted of 96-well-microtiter plates containing 200 μl of tap water per well and 5-15 freshly hatched A. aegypti larvae. The active compounds were formulated using a solution containing 75% (v/v) water and 25% (v/v) DMSO. Different concentrations of formulated compounds or mixtures were sprayed onto the insect diet at 2.5 μl, using a custom-built micro atomizer, at two replications.

After application, microtiter plates were incubated at 28±1° C., 80±5% RH for 2 days. Larval mortality was then visually assessed.

In this test, compounds I-3, I-4, I-5, I-6, I-7, I-9, I-10, I-11, I-12, I-13, I-14, I-15, I-16, I-17, I-18, I-19, I-20, I-21, I-22, I-42, I-73, I-74, I-78, I-79, I-80, I-81, I-82, and I-84, resp., at 2500 ppm showed at least 75% mortality in comparison with untreated controls.

In this test, compounds I-3, I-4, I-6, I-13, I-14, I-15, I-16, I-17, I-18, I-19, I-20, I-21, I-22, I-23, I-25, I-26, I-27, I-28, I-29, I-31, I-32, I-33, I-34, I-37, I-38, I-40, I-42, I-43, I-44, I-45, I-46, I-47, I-48, I-49, I-50, I-51, I-52, I-53, I-54, I-55, I-58, I-59, I-60, I-62, I-63, I-65, I-66, I-68, I-69, I-70, I-71, I-72, I-75, I-76, I-77, I-83, II-1, and III-1, resp., at 800 ppm showed at least 75% mortality in comparison with untreated controls.

B.5. Diamond Back Moth (Plutella xylostella)

For evaluating control of tobacco budworm (Plutella xylostella) the test unit consisted of 96-well-microtiter plates containing an insect diet and 15-25 P. xylostella eggs.

The compounds or mixtures were formulated using a solution containing 75% water and 25% DMSO. Different concentrations of formulated compounds or mixtures were sprayed onto the insect diet at 5 μl, using a custom-built micro atomizer, at two replications.

After application, microtiter plates were incubated at 28≅1° C., 80±5% RH for 5 days. Egg and larval mortality was then visually assessed.

In this test, compounds I-2, I-10, I-11, I-12, I-13, I-14, I-15, I-16, I-17, I-18, I-19, I-20, I-21, I-22, I-42, I-73, I-74, I-78, I-79, I-80, I-81, and I-82, resp., at 2500 ppm showed at least 75% mortality in comparison with untreated controls.

In this test, compounds I-2, I-11, I-12, I-13, I-14, I-15, I-16, I-17, I-18, I-19, I-20, I-21, I-22, I-23, I-25, I-26, I-28, I-29, I-32, I-33, I-34, I-37, I-38, I-40, I-42, I-43, I-44, I-45, I-46, I-47, I-48, I-50, I-51, II-1, III-1, and III-2, resp., at 800 ppm showed at least 75% mortality in comparison with untreated controls.

The beneficial activity of the compounds according to the invention over structurally close compounds known from WO2022/171472 was demonstrated by the following comparative experiments:

C.1. Western Flower Thrips (Frankliniella occidentalis)

The active compounds were formulated by a Tecan liquid handler in 100% cyclohexanone as a 10,000 ppm solution supplied in tubes. The 10,000 ppm solution was serially diluted in 100% cyclohexanone to make interim solutions. These served as stock solutions for which final dilutions were made by the Tecan in 50% acetone:50% water (v/v) into 10 or 20 ml glass vials. A nonionic surfactant (Kinetic®) was included in the solution at a volume of 0.01% (v/v). The vials were then inserted into an automated electrostatic sprayer equipped with an atomizing nozzle for application to plants/insects. Small (˜2″ in height) cotton plants were sprayed with test compounds at concentrations ranging from 300 to 0.01 ppm in acetone/water through the automated VPS. After drying, cotton leaves were removed, and circular leaf discs (˜1 cm diameter) were punched from the treated surface and transferred to clean 20 mL scintillation vials. Ten Western flower thrips (FRANOC) were aspirated into each scintillation vial. The vials with the leaf discs and thrips were kept in an upright incubator at 25° C. and 50% relative humidity with a 14:10 light:dark photoperiod. Each treatment was replicated twice. Thrips mortality was assessed at 2 DAT (days after treatment), counting all thrips both dead and alive.

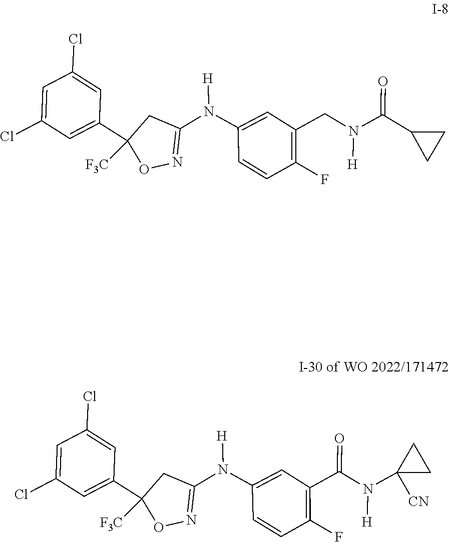 [SMILES: O=C(NCc1cc(NC2=NOC(c3cc(Cl)cc(Cl)c3)(C(F)(F)F)C2)ccc1F)C1CC1 ; N#CC1(NC(=O)c2cc(NC3=NOC(c4cc(Cl)cc(Cl)c4)(C(F)(F)F)C3)ccc2F)CC1]

In this test, compound I-8 at 100 ppm showed 100% mortality, whereas compound I-30 of WO2022/171472 at 100 ppm showed 19% mortality in comparison with untreated controls.

C.2. Southern Armyworm (Spodoptera eridania), 2nd Instar Larvae

The active compounds were formulated by a Tecan liquid handler in 100% cyclohexanone as a 10,000-ppm solution supplied in tubes. The 10,000-ppm solution was serially diluted in 100% cyclohexanone to make interim solutions. These served as stock solutions for which final dilutions were made by the Tecan in 50% acetone:50% water (v/v) into 10 or 20 ml glass vials. A nonionic surfactant (Kinetic®) was included in the solution at a volume of 0.01% (v/v). The vials were then inserted into an automated electrostatic sprayer equipped with an atomizing nozzle for application to plants/insects. Lima bean plants (variety Sieva) were grown 2 plants to a pot and selected for treatment at the 1st true leaf stage. Test solutions were sprayed onto the foliage by an auto-mated electrostatic plant sprayer equipped with an atomizing spray nozzle. The plants were dried in the sprayer fume hood and then removed from the sprayer. Each pot was placed into perforated plastic bags with a zip closure. Ten to 11 armyworm larvae were placed into the bag and the bags zipped closed. Test plants were maintained in a growth room at about 25° C. and about 20-40% relative humidity for 4 days, avoiding direct exposure to fluorescent light (14:10 light:dark photoperiod) to prevent trapping of heat inside the bags. Mortality and reduced feeding were assessed 4 days after treatment, compared to untreated control plants.

In this test, compound I-8 at 1 ppm showed 80% mortality, whereas compound I-30 of WO2022/171472 at 1 ppm showed 0% mortality in comparison with untreated controls.

1. A compound of formula I 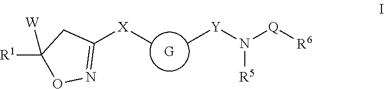
wherein
R¹ is C₁-C₂-haloalkyl;
W is phenyl, or pyridyl; wherein W is unsubstituted, partially or fully substituted with R²;
R² is halogen, OR²¹, NR²²R²³, CN, NO₂, Si(CH₃)₃, SF₅, C₁-C₄-alkyl, C₁-C₄-haloalkyl, C₃-C₆-cycloalkyl, C₃-C₆-halocycloalkyl, C₂-C₄-alkenyl, C₂-C₄-haloalkenyl, C₂-C₄-alkynyl, C₂-C₄-haloalkynyl, C₁-C₃-alkoxy, C₁-C₃-haloalkoxy, C₁-C₃-alkyl-S(O)ₘ, C₁-C₃-haloalkyl-S(O)ₘ, C₃-C₆-cycloalkyl-S(O)ₘ, C₁-C₃-alkoxy-C₁-C₄-alkyl, C₁-C₃-haloalkoxy-C₁-C₄-alkyl, C₁-C₃-alkyl-S(O)ₘ—C₁-C₄-alkyl, C₁-C₃-haloalkyl-S(O)ₘ—C₁-C₄-alkyl;
which groups are unsubstituted, partially or fully substituted with R²¹¹;
R²¹ H, C₁-C₆-alkyl, C₂-C₆-alkenyl, C₂-C₆-alkynyl, C₃-C₈-cycloalkyl, Si(C₁-C₄-alkyl)₃, C₁-C₃-alkyl-S(O)ₘ, C₃-C₆-cycloalkyl-S(O)ₘ, S(O)ₘR²⁴, which groups are unsubstituted, partially or fully substituted with R²¹¹;
R²², R²³ H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, which are unsubstituted or partially or fully substituted with R²²¹; or
C₁-C₆-alkyl-C(═O)OR²⁴, C₁-C₆-alkyl-C(═U)N(R^{25a})R^{25b}, C₁-C₆-alkyl-C(═NR²⁵)N(R^{25a})R^{25b},
S(O)ₘR²⁴, S(O)ₘN(R^{25a})R^{25b}, C(═U)R²⁶, C(═O)OR²⁴, C(═U)N(R^{25a})R^{25b}, C(═S)SR²⁴, C(═NR²⁵)R²⁶;
C₃-C₈-cycloalkyl, C₃-C₈-halocycloalkyl;
R²² and R²³ form together with the nitrogen atom they are bonded to a 3-, 4-, 5-, or 6-membered fully unsaturated heterocycle, which heterocycle may additionally contain one heteroatom selected from N, O, and S(O)ₘ as ring members, and which heterocycle is unsubstituted or partially or fully substituted with R²²²; or
R²² and R²³ together form a group ═C(R²¹)₂, ═S(O)ₘ(R²⁴)₂,
═S(O)ₘR²⁴N(R^{25a})R^{25b};
U is O or S;
R²⁴ is H, Si(C₁-C₄-alkyl)₃, C₁-C₆-alkyl, C₂-C₆-alkenyl, C₂-C₆-alkynyl, which are unsubstituted or partially or fully halogenated and/or substituted with C₃-C₄-cycloalkyl, C₁-C₄-alkoxy, C₁-C₄-haloalkoxy, S(O)ₘ—C₁-C₄-alkyl, and oxo;
C₃-C₈-cycloalkyl which is unsubstituted or partially or fully halogenated and/or substituted with C₁-C₄-alkyl, C₃-C₄-cycloalkyl, C₁-C₄-alkoxy, C₁-C₄-haloalkoxy, S(O)ₘ—C₁-C₄-alkyl, and oxo;
phenyl, benzyl, pyridyl and phenoxy, which are unsubstituted or partially or fully halogenated and/or substituted with C₁-C₆-alkyl, C₁-C₆-haloalkyl, C₁-C₆-alkoxy, C₁-C₆-haloalkoxy, C₁-C₆-alkylthio, C₁-C₆-haloalkylthio, and (C₁-C₆-alkoxy)carbonyl;
R²⁵ is H, C₁-C₆-alkoxy, C₁-C₆-haloalkoxy, C(═U)R²⁶, C(═O)OR²⁴,
C(═O)NH(C₁-C₄-alkyl),
C(═O)N(C₁-C₄-alkyl)₂, S(O)ₘR²⁴, S(O)ₘ—C₁-C₄-alkyl, S(O)ₘ—C₁-C₄-haloalkyl, S(O)ₘ—C₃-C₆-cycloalkyl, Si(C₁-C₄-alkyl)₃, NR²⁴C(═O)—C₁-C₄-alkyl, NR²⁴C(═O)—C₃-C₆-cycloalkylalkyl, CR²⁴N═OR²⁴, CR²⁶N═OR²⁴,
C₁-C₆-alkyl, C₃-C₆-alkenyl, C₃-C₆-alkynyl, which are unsubstituted or partially or fully halogenated and/or substituted with CN, C₁-C₄-alkoxy, C₁-C₄-haloalkoxy, S(O)ₘ—C₁-C₄-alkyl, S(O)ₘ—C₁-C₄-haloalkyl, C₃-C₆-cycloalkyl which is unsubstituted or substituted with 1 or 2 halogen and/or CN; phenyl, or 4-, 5-, 6-membered saturated, partially or fully unsaturated heterocyclyl comprising 1, 2 or 3 heteroatoms selected from N, O, and S(O)ₘ as ring members, which rings are unsubstituted or partially or fully substituted with halogen, CN, C₁-C₄-alkyl, C₁-C₄-haloalkyl, C₁-C₃-alkoxy, C₁-C₃-haloalkoxy, C₁-C₂-alkyl-C₁-C₂-alkoxy, C₁-C₃-alkylthio, C₁-C₃-haloalkylthio, C(═O)—C₁-C₄-alkoxy; and oxo;
C₃-C₈-cycloalkyl which is unsubstituted or partially or fully halogenated and/or substituted with CN, C₁-C₄-alkyl, C₁-C₄-alkoxy, C₁-C₄-haloalkyl, C₁-C₄-haloalkoxy, S(O)ₘ—C₁-C₄-alkyl, S(O)ₘ—C₁-C₄-haloalkyl, C(═O)NH(C₁-C₄-alkyl), C(═O)N(C₁-C₄-alkyl)₂, phenyl, or 5-, 6-membered heteroaryl containing 1, 2 or 3 heteroatoms selected from N, O, and S(O)ₘ as ring members, which rings are unsubstituted or partially or fully substituted with halogen, CN, C₁-C₄-alkyl, C₁-C₄-haloalkyl, C₁-C₃-alkoxy, C₁-C₃-haloalkoxy, C₁-C₂-alkyl-C₁-C₂-alkoxy, C₁-C₃-alkylthio, C₁-C₃-haloalkylthio;
phenyl, benzyl, phenoxy, 4-, 5-, 6-membered saturated, partially or fully unsaturated heterocyclyl comprising 1, 2 or 3 heteroatoms selected from N, O, and S(O)ₘ as ring members,
which groups are unsubstituted or partially or fully halogenated and/or substituted with halogen, CN, NO₂, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, and (C₁-C₆-alkoxy)carbonyl; and a 3-, 4-, 5- or 6-membered saturated, partially or fully unsaturated heterocycle comprising 1, 2 or 3 heteroatoms selected from N, O, and S(O)ₘ as ring members, where the heterocycle is optionally substituted with one or more R²²²;
R^{25a} and R^{25b} have the meanings given for R²⁵; or
R^{25a} and R^{25b} present on the same nitrogen atom may together form ═C(R²⁶)₂, ═S(O)ₘ(R²⁴)₂, or ═S(O)ₘR²⁴N(R^{25a})R^{25b}; or
R^{25a} and R^{25b}, together with the nitrogen atom to which they are bound, form a 3-, 4-, 5-, 6- or 7-membered saturated, partially unsaturated or fully unsaturated heterocyclic ring, wherein the heterocyclic ring may additionally contain 1 or 2 heteroatoms or heteroatom groups selected from N, O, and S(O)ₘ as ring members, which heterocycle is unsubstituted or substituted with one or more substituents halogen, CN, C₁-C₄-alkyl, C₁-C₄-haloalkyl, C₁-C₄-alkoxy, C₁-C₄-haloalkoxy, and oxo; or R^{25a} and R^{25b}, together with the nitrogen atoms to which they are bound in the group C(═NR²⁵)N(R^{25a})R^{25b}, form a 3-, 4-, 5-, 6- or 7-membered saturated, partially unsaturated or fully unsaturated heterocyclic ring, wherein the heterocyclic ring may additionally contain 1 or 2 heteroatoms or heteroatom groups selected from N, O, and S(O)ₘ as ring members, which heterocycle is unsubstituted or substituted with one or more substituents halogen, CN, C₁-C₄-alkyl, C₁-C₄-haloalkyl, C₁-C₄-alkoxy, C₁-C₄-haloalkoxy, and oxo;
R²⁶ is H, CN, OH, SH, C₁-C₆-alkyl, C₂-C₆-alkenyl, C₂-C₆-alkynyl, C₃-C₈ cycloalkyl, which are unsubstituted, partially or fully halogenated and/or substituted with 1, 2, or 3 groups CN, C₁-C₄-alkyl, C₃-C₄-cycloalkyl, C₁-C₄-haloalkyl, C₁-C₄-alkoxy, C₁-C₄-haloalkoxy, S(O)ₘ—C₁-C₄-alkyl, S(O)ₘ—C₁-C₄-haloalkyl, phenyl, or 3-, 4-, 5-, 6- or 7-membered saturated, partially or fully unsaturated heterocycle containing 1, 2 or 3 heteroatoms N, O, and S(O)ₘ as ring members, which rings are unsubstituted or partially or fully substituted with halogen, CN, C₁-C₄-alkyl, C₁-C₄-haloalkyl, C₁-C₃-alkoxy, C₁-C₃-haloalkoxy, C₁-C₂-alkyl-C₁-C₂-alkoxy, C₁-C₃-alkylthio, C₁-C₃-haloalkylthio; and oxo;
C₁-C₆-alkoxy, C₁-C₆-haloalkoxy, S(O)ₘ—C₁-C₆-alkyl, S(O)ₘ—C₁-C₆-haloalkyl, Si(C₁-C₄-alkyl)₃, C(═O)N(R^{25a})R^{25b},
phenyl, benzyl, phenoxy, a 3-, 4-, 5-, 6- or 7-membered saturated, partially or fully unsaturated heterocycle containing 1, 2 or 3 heteroatoms selected from N, O, and S(O)ₘ as ring members, which rings are unsubstituted or partially or fully halogenated and/or substituted with R²²²;
R²¹¹ halogen, CN, C₁-C₄-alkoxy, C₁-C₄-haloalkoxy, C₃-C₄-alkenyloxy, C₃-C₄-haloalkenyloxy, C₃-C₄-alkynyloxy, C₃-C₄-haloalkynyloxy, C₁-C₄-alkyl-S(O)ₘ, C₁-C₄-haloalkyl-S(O)ₘ, C₃-C₄-alkenyl-S(O)ₘ, C₃-C₄-haloalkenyl-S(O)ₘ, C₃-C₄-alkynyl-S(O)ₘ, C₃-C₄-haloalkynyl-S(O)ₘ, and oxo;
C₃-C₈-cycloalkyl, C₃-C₅-halocycloalkyl, C₃-C₈-cycloalkenyl, C₃-C₈-halocycloalkenyl;
R²²¹ CN, NO₂, OH, SH, SCN, SF₅, Si(C₁-C₄-alkyl)₃, C₁-C₆-alkoxy, C₁-C₆-haloalkoxy, C₁-C₆-alkyl-S(O)ₘ, C₁-C₆-haloalkyl-S(O)ₘ,
C(═O)N(R^{25a})R^{25b};
C₃-C₈-cycloalkyl which is unsubstituted, partially or fully halogenated and/or partially or fully substituted with C₁-C₄-alkyl, C₃-C₄-cycloalkyl, C₁-C₄-alkoxy, C₁-C₄-haloalkoxy, and oxo; or
two R²²¹ present on the same carbon atom of an alkyl, alkenyl, alkynyl or cycloalkyl group may together be ═O, ═CH(C₁-C₄-alkyl), ═C(C₁-C₄-alkyl)₂, ═N(C₁-C₆-alkyl), or ═NO(C₁-C₆-alkyl);
R²²² is halogen, NO₂, CN, OH, SH, C₁-C₆-alkoxy, C₁-C₆-haloalkoxy, S(O)ₘ—C₁-C₄-alkyl, S(O)ₘ—C₁-C₄-haloalkyl, C₁-C₄-alkylcarbonyl, C₁-C₄-haloalkylcarbonyl, C₁-C₄-alkoxycarbonyl, C₁-C₄-haloalkoxycarbonyl, N(R^{25a})R^{25b}, C(═O)NR^{25a}R^{25b}, Si(C₁-C₄-alkyl)₃;
C₁-C₄-alkyl, C₂-C₆-alkenyl, C₂-C₆-alkynyl, which are unsubstituted or partially or fully halogenated and/or substituted with CN, C₃-C₄-cycloalkyl, C₁-C₄-alkoxy, C₁-C₄-haloalkoxy, S(O)ₘ—C₁-C₄-alkyl, and oxo;
C₃-C₈-cycloalkyl which is unsubstituted or partially or fully halogenated and/or substituted with CN, C₁-C₄-alkyl, C₃-C₄-cycloalkyl, C₁-C₄-alkoxy, C₁-C₄-haloalkyl, C₁-C₄-haloalkoxy, S(O)ₘ—C₁-C₄-alkyl, and oxo; or
two R²²² present together on the same atom of an unsaturated or partially unsaturated ring may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl; or
two R²²² on two adjacent carbon atoms form together with the carbon atoms they are bonded to a 4-, 5-, 6-, 7- or 8-membered saturated, partially unsaturated or maximally unsaturated ring, wherein the ring may contain 1 or 2 heteroatoms or heteroatom groups selected from N, O, and S(O)ₘ as ring members, and wherein the ring is optionally substituted with one or more groups C₁-C₄-alkyl, C₁-C₄-haloalkyl, C₁-C₄-alkoxy, and/or C₁-C₄-haloalkoxy;
m is 0, 1, or 2;
X is NR³, or O;
R³ is H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, OR²¹, C₁-C₆-alkylthio, C₁-C₆-haloalkylthio, which are unsubstituted or partially or fully substituted with R³¹; or
C₁-C₆-alkyl-C(═O)OR²⁴, C₁-C₆-alkyl-C(═U)N(R^{25a})R^{25b},
C₁-C₆-alkyl-C(═NR²⁵)N(R^{25a})R^{25b}, C₁-C₆-alkyl-OC(═O)OR²⁴,
N(R^{25a})R^{25b}, S(O)ₘR²⁴, S(O)ₘN(R^{25a})R^{25b}, C(═U)R²⁶, C(═O)OR²⁴,
C(═U)N(R^{25a})R^{25b}, C(═S)SR²⁴, C(═NR²⁵)R²⁶;
C₃-C₈-cycloalkyl, C₃-C₈-halocycloalkyl, phenyl, a 3-, 4-, 5-, 6- or 7-membered saturated or partially unsaturated heterocycle comprising 1, 2, 3 or 4 heteroatoms selected from N, O, and S(O)ₘ as ring members, or a 5- or 6-membered hetaryl comprising 1, 2, 3 or 4 heteroatoms selected from N, O, and S(O)ₘ as ring members, which rings are unsubstituted or partially or fully substituted with R³²;
R³¹ halogen, CN, NO₂, OH, SH, SCN, SF₅, Si(C₁-C₄-alkyl)₃, N(R^{25a})R^{25b}, C₁-C₆-alkoxy, C₁-C₆-haloalkoxy, C₁-C₆-alkyl-S(O)ₘ, C₁-C₆-haloalkyl-S(O)ₘ, C(═O)N(R^{25a})R^{25b};
C₃-C₈-cycloalkyl which is unsubstituted, partially or fully halogenated and/or partially or fully substituted with CN, C₁-C₄-alkyl, C₃-C₄-cycloalkyl, C₁-C₄-alkoxy, C₁-C₄-haloalkyl, C₁-C₄-haloalkoxy, and oxo;
S(O)ₘR²⁴, S(O)ₘN(R^{25a})R^{25b}, C(═U)R²⁶, C(═O)OR²⁴, C(═U)N(R^{25a})R^{25b}, C(═S)SR²⁴, C(═NR²⁵)R²⁶;
phenyl, benzyl, phenoxy, or 3-, 4-, 5-, 6- or 7-membered saturated, partially, or fully unsaturated heterocycle containing 1, 2, or 3 heteroatoms selected from N, O, S(O)ₘ as ring members, wherein the rings are unsubstituted or partially or fully substituted with R²²²; or
two R³¹ present on the same carbon atom of an alkyl, alkenyl, alkynyl or cycloalkyl group may together be ═O, ═CH(C₁-C₄-alkyl), ═C(C₁-C₄-alkyl)₂, ═N(C₁-C₆-alkyl), or ═NO(C₁-C₆-alkyl);
R³² is a group as defined in R³¹ or selected from C₁-C₆-alkyl, C₂-C₆-alkenyl, and C₂-C₆-alkynyl, which groups are unsubstituted, partially or fully halogenated and/or substituted with one or two CN, C₃-C₄-cycloalkyl, C₁-C₄-alkoxy, C₁-C₄-haloalkoxy, or oxo;
G is phenyl, or 6-membered heteroaryl comprising as ring members 1 or 2 N-atoms; wherein G is unsubstituted, partially, or fully substituted with R⁴;
R⁴ is as defined for R²;
R⁵ is a group as defined for R³;
Y is a direct bond or C(R^{4a})(R^{4b});
R^{4a} and R^{4b} are H, halogen, CN, NO₂, C₁-C₄-alkyl, C₁-C₄-haloalkyl, C₃-C₆-cycloalkyl unsubstituted or substituted with CN or halogen;
R⁴ and R^{4a} and/or R^{4b} together form a 3-, 4-, 5-, 6- or 7-membered saturated, partially, or fully unsaturated heterocycle, which may contain 1 or 2 heteroatoms selected from N, O, and S(O)ₘ as ring members, and wherein the ring is unsubstituted or substituted with one or more groups R²;
Q is C(═U) or S(O)ₘ;
R⁶ is as defined for R²⁵; or
R⁵ and R⁶ together form a 4-, 5-, 6- or 7-membered saturated, or partially unsaturated heterocycle, which additionally to the N(R⁵) may contain 1 or 2 heteroatoms selected from N, O, and S(O)ₘ as ring members, and wherein the ring is unsubstituted or substituted with one or more groups R²;
and the N-oxides, stereoisomers and agriculturally or veterinarily acceptable salts thereof.

2. The compound of formula I according to claim 1, wherein W is phenyl, which is substituted with one to three groups halogen, halomethyl, halomethoxy, and/or halomethyl —S(O)ₘ.

3. The compound of formula I according to claim 1, wherein
Y is C(R^{4a})(R^{4b}), preferably CH₂.

4. The compound of formula I according to claim 1, wherein
Q is C(═O).

5. The compound of formula I according to claim 1, wherein
X is NR³, and
R³ is H, alkoxycarbonyl, C₁-C₆-alkyl, which alkyl is unsubstituted or substituted with CN, cycloalkyl, alkoxy.

6. The compound of formula I according to claim 1, wherein X is O.

7. The compound of formula I according to claim 1, wherein G is a group G1, G2, G3, or G4, 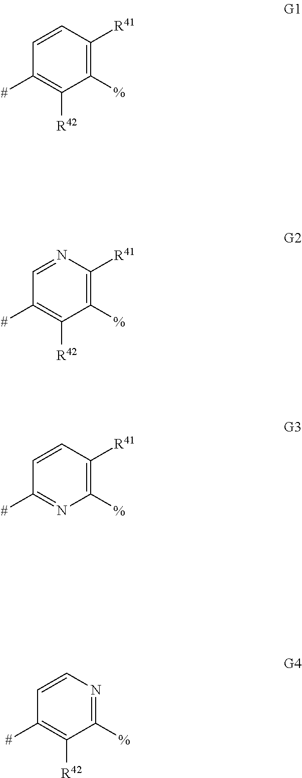
wherein #is the bond to X, % is the bond to Y, and R⁴¹ and R⁴² are H or a group R², preferably H, or halogen, CN, halomethyl, or halomethoxy.

8. The compound of formula I according to claim 7, wherein G is G1 and R⁴² is H.

9. The compound of formula I according to claim 1, wherein
R¹ is halomethyl;
W is phenyl, which is partially or fully substituted with R²;
R² is halogen, CN, OH, C₁-C₄-alkoxy, OR²¹, C₁-C₄-haloalkyl, C₁-C₃-alkoxy, C₁-C₃-haloalkoxy, C₁-C₃-alkyl-S(O)ₘ, C₁-C₃-haloalkyl-S(O)ₘ, C₁-C₃-alkoxy-C₁-C₄-alkyl;
X is NR³ or O;
R³ is H, C₁-C₄-alkyl, C₁-C₄-alkoxy-C₁-C₄-alkyl, C₁-C₄-alkoxycarbonyl, C₃-C₆-cycloalkyl-C₁-C₄-alkyl, C₂-C₄-alkenyl, C₂-C₄-alkynyl,
G is phenyl, which is unsubstituted, partially, or fully substituted with R⁴;
preferably a group G1, wherein R⁴¹ and R⁴² are H, halogen, C₁-C₄-alkyl, C₁-C₄-haloalkyl, C₁-C₄-alkoxy, C₁-C₄-haloalkoxy;
R⁴ is H, halogen C₁-C₄-alkyl, C₁-C₄-haloalkyl, C₁-C₄-alkoxy, C₁-C₄-haloalkoxy; or R⁴ and R^{4a}/R⁴¹ together form a 5-, or 6-membered saturated or unsaturated carbo- or heterocycle, which heterocycle may contain 1 or 2 N as ring members;
Y is a direct bond or CR^{4a}R^{4b};
Q is C(═O) or SO₂;
R⁵ H,
R⁶ C₁-C₄-alkyl, C₁-C₄-haloalkyl, C₃-C₆-cycloalkyl, C₃-C₆-cycloalkyl-C₁-C₄-alkyl, C₃-C₆-cycloalkenyl, C₁-C₄-alkoxy-C₁-C₄-alkyl, C₁-C₄-alkyl-S(O)ₘ—C₁-C₄-alkyl, NR^{H}C(═O)—C₁-C₄-alkyl, NR^{H}C(═O)—C₃-C₆-cycloalkyl, CR^{H}N═OR²⁴, CR^{H}N═OR²⁴, 4-, 5-, or 6-membered saturated or partially or fully unsaturated heterocycle containing 1, 2 or 3 heteroatoms selected from O, N, S as ring members, wherein S may be oxidized, wherein R^{H} is H, CN or C₁-C₄-alkyl,
R⁶ being unsubstituted or substituted with CN, C₁-C₄-alkoxy, C₁-C₄-haloalkoxy, S(O)ₘ—C₁-C₄-alkyl, S(O)ₘ—C₁-C₄-haloalkyl, C₃-C₆-cycloalkyl which is unsubstituted or substituted with 1 or 2 halogen and/or CN.

10. The compound of formula I according to claim 1, wherein R¹ is CF₃.

11. The compound of formula I according to claim 1, which correspond to formula I.A

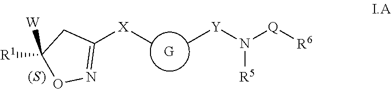

12. An agricultural or veterinary composition comprising at least one compound according to claim 1 and/or at least one agriculturally or veterinarily acceptable salt thereof, and at least one inert liquid and/or solid agriculturally or veterinarily acceptable carrier.

13. An agricultural composition for combating animal pests comprising at least one compound as defined in claim 1 and at least one inert liquid and/or solid acceptable carrier and, if optionally, at least one surfactant.

14. A method for combating or controlling an invertebrate pest comprising contacting said pest or its food supply, habitat or breeding grounds with a pesticidally effective amount of at least one compound as defined in claim 1.

15. A method for protecting growing plants from attack or infestation by an invertebrate pest comprising contacting a plant, or soil or water in which the plant is growing, with a pesticidally effective amount of at least one compound as defined in claim 1.

16. A seed comprising a compound as defined in claim 1, or enantiomers, diastereomers or salts thereof, in an amount of from 0.1 g to 10 kg per 100 kg of seed.

17. A method for treating or protecting an animal from infestation or infection by an invertebrate pest comprising bringing the animal in contact with a pesticidally effective amount of at least one compound of the formula I as defined in claim 1, a stereoisomer thereof and/or at least one veterinarily acceptable salt thereof.
